SEQUENCE LISTING (76 sequences)

SEQ ID NO: 1 - protein, 418 aa, SARS-Cov2
SDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 2 - protein, 48 aa, synthetic construct
  REGION 1..48: note = hybrid human chicken C4Bp
KKQGDADVCGEVAYIQSVVSDCHVPTAELRTLLEIRKLFLEIQKLKVE

SEQ ID NO: 3 - skipped

SEQ ID NO: 4 - protein, 7 aa, synthetic construct
  REGION 1..7: note = Postively charged peptide
GRRRRRS

SEQ ID NO: 5 - protein, 55 aa, synthetic construct
  REGION 1..55: note = OVX313
KKQGDADVCGEVAYIQSVVSDCHVPTAELRTLLEIRKLFLEIQKLKVEGRRRRRS

SEQ ID NO: 6 - protein, 475 aa, synthetic construct
  REGION 1..475: note = OVX033 amino acid sequence without methionine
SDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAGSKKQGDADVCGEVAYIQSVVSDCHVPTAELRTLLEIRKLFLEIQKLKVEGRRRRRS

SEQ ID NO: 7 - protein, 31 aa, synthetic construct
  REGION 1..31: note = tPA signal sequence
DAMKRGLCCVLLLCGAVFVSPSQEIHARFRR

SEQ ID NO: 8 - protein, 497 aa, Influenza A virus
ASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTHMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRNPGNAEFEDLTFLARSALILRGSVAHKSCLPACVYGPAVASGYDFEREGYSLVGIDPFRLLQNSQVYSLIRPNENPAHKSQLVWMACHSAAFADLRVLSFIKGTKVVPRGKLSTRGVQIASNENMETMESSTLELRSRYWAIRTRSGGNTNQQRASAGQISIQPTFSVQANLPFDRTTVMAAFTGNTEGRTSDMRTEIIRMMESARPEDVSFQGRGVFELSDEKAASPIVPSFDMSNEGSYFFGDNAEEYDN

SEQ ID NO: 9 - protein, 9 aa, Influenza A virus
ASNENMETM

SEQ ID NO: 10 - protein, 15 aa, Influenza A virus
QVYSLIRPNENPAHK

SEQ ID NO: 11 - protein, 19 aa, Influenza A virus
RGVQIASNENMETMESSTL

SEQ ID NO: 12 - protein, 25 aa, Influenza A virus
LLQNSQVYSLIRPNENPAHKSQLVW

SEQ ID NO: 13 - RNA, 1494 nt, Influenza A virus
  source 1..1494: mol_type = mRNA
gcgactaagggcacgaaacgcagctacgaacaaatggaaaccgacggtgagcgtcaaaatgcaaccgaaatccgcgctagcgtcggcaagatgatcgacggcatcggccgtttttacattcagatgtgcaccgagctgaagctgagcgattacgagggtcgtctgattcagaatagcttgacgatcgagcgtatggtgttgagcgcgttcgatgagcgccgcaacaaatatctggaagaacatccgagcgccggtaaagatccgaagaaaaccggtggccctatctaccgtcgtgttgatggcaagtggcgtcgcgagctgattctgtatgacaaagaagaaattcgccgtatttggcgccaggcgaataatggtgacgacgcgaccgcgggtttaacgcacatgatgatctggcattccaacctgaacgatgcgacgtatcaacgtacccgtgcgctggtgcgtaccggcatggacccacgtatgtgctcgctgatgcaaggttccaccctgcctcgtcgtagcggtgctgccggtgcggcagtgaaaggtgtcggcacgatggtcatggaacttatccgcatgattaagcgcggtatcaatgatcgtaatttctggcgcggtgagaatggtcgtcgtacccgtattgcgtatgagcgtatgtgcaacattctgaagggtaaattccagaccgcggcacagcgtacgatggtcgaccaagttcgcgagtctcgtaacccgggcaatgctgagtttgaagatctgattttcctggcgcgtagcgccctgattctgcgtggctcggttgcgcacaaatcttgtctgccggcctgcgtctatggtagcgcggtggcatccggttacgactttgagcgtgagggttatagcttggttggcattgacccgtttcgcctgctgcagaacagccaggtgtacagcctgatccgtccaaatgagaacccggcacacaagtcccaactggtttggatggcatgtcatagcgcggctttcgaagatctgcgtgtgtctagctttatccgcggtaccaaagttgtgccgcgtggcaagctgagcacgcgtggtgtgcaaatcgccagcaacgaaaacatggaaaccatggaatcttcaaccctggagctgcgtagccgttactgggcgattcgcacccgcagcggtggcaataccaaccagcaacgtgcgagcagcggccagatcagcattcaaccgacttttagcgttcagcgtaatctgccgttcgaccgcccgacgatcatggcagcctttaccggtaacaccgagggtcgcactagcgacatgcgcaccgaaatcattcgcctgatggagagcgcccgtccggaagatgtcagcttccagggtcgtggtgttttcgagctgagcgacgagaaagcgacctccccgatcgtcccgagctttgacatgtctaacgagggcagctactttttcggtgataatgcagaagagtacgataactaa

SEQ ID NO: 14 - RNA, 27 nt, Influenza A virus
  source 1..27: mol_type = mRNA
gccagcaacgaaaacatggaaaccatg

SEQ ID NO: 15 - RNA, 45 nt, Influenza A virus
  source 1..45: mol_type = mRNA
caggtgtacagcctgatccgtccaaatgagaacccggcacacaag

SEQ ID NO: 16 - RNA, 57 nt, Influenza A virus
  source 1..57: mol_type = mRNA
cgtggtgtgcaaatcgccagcaacgaaaacatggaaaccatggaatcttcaaccctg

SEQ ID NO: 17 - RNA, 75 nt, Influenza A virus
  source 1..75: mol_type = mRNA
ctgctgcagaacagccaggtgtacagcctgatccgtccaaatgagaacccggcacacaagtcccaactggtttgg

SEQ ID NO: 18 - protein, 101 aa, synthetic construct
  REGION 1..101: note = Fusion protein with CD8 and CD4 T cell epitopes and OVX313
RGVQIASNENMETMESSTLLLQNSQVYSLIRPNENPAHKSQLVWGSKKQGDADVCGEVAYIQSVVSDCHVPTAELRTLLEIRKLFLEIQKLKVEGRRRRRS

SEQ ID NO: 19 - RNA, 306 nt, synthetic construct
  misc_feature 1..306: note = Coding sequence of fusion protein with CD8 and CD4 epitopes of NP fused to OVX313
  source 1..306: mol_type = other RNA
cgtggtgtgcaaatcgccagcaacgaaaacatggaaaccatggaatcttcaaccctgctgctgcagaacagccaggtgtacagcctgatccgtccaaatgagaacccggcacacaagtcccaactggtttggggcagcaagaaacagggtgatgctgacgtgtgcggcgaagtggcatatatccagagcgtcgtgagcgattgtcacgttccgacggcagagttgcgcacgctgttggaaatccgtaagctgttcttggagattcaaaagctcaaagttgagggtcgtcgtcgcagacgttcctaa

SEQ ID NO: 20 - RNA, 1428 nt, SARS-Cov2
  source 1..1428: mol_type = mRNA
agcgacaatggcccacagaatcaacgtaatgcaccgagaatcacctttggcggcccgagcgatagcacgggtagcaaccagaacggcgagcgcagcggtgcgcgttctaaacagcgccgtccgcaaggtctgccgaataacacggcgtcgtggtttacggcactgacccagcatggtaaggaagatttgaagtttccgcgtggccagggtgtcccgatcaataccaacagctcacctgatgaccagatcggctattatcgtcgcgccacgcgccgtatccgcggtggcgatggcaagatgaaagacttaagcccgcgttggtacttctattacctgggtacgggtccagaggctggtttgccgtatggtgctaacaaagacggcattatctgggtggcgaccgagggtgccctgaacaccccgaaagatcacattggtactcgtaaccctgcgaacaacgccgcgattgtgctgcaactgccgcagggtaccaccctgccgaagggtttctatgcagagggcagccgtggtggcagccaggcgagcagccgtagcagcagccgttcccgcaattctagccgcaatagcacgccgggttccagccgtggtacgtccccggcacgtatggcaggcaatggtggcgatgcggcactggcgctgctgttgctggaccgcctgaaccaactggagagcaagatgagcggcaagggtcagcagcagcaaggtcagactgtgaccaaaaagtccgcggcagaggccagcaagaaaccacgtcagaaacgtaccgcgaccaaagcgtacaatgttacccaggccttcggtcgccgtggccctgagcaaacccaaggtaatttcggtgaccaagaactgatccgccagggcacggactacaagcattggccgcagattgcgcagtttgcaccgagcgctagcgcgttcttcggtatgtcccgcattggtatggaagtgaccccgtctggcacctggctgacctacactggtgcaatcaaattggacgataaagacccgaactttaaagaccaagtcatcctcctgaacaagcacattgatgcgtacaagaccttcccgccgacggaaccgaaaaaagacaaaaagaaaaaagctgacgaaacccaagcattgccgcaacgtcaaaagaaacagcaaacggttaccctgcttccggcagcggatctggacgattttagcaagcaactgcaacaatctatgtcgagcgcggactccacccaggccggtagcaagaaacagggcgatgccgatgtctgcggcgaagttgcgtacattcagagcgtcgtgagcgactgtcacgttccgacggctgagctgcgtaccctgctggaaattcgtaagctgttcctggaaatccaaaagctgaaagttgagggtcgccgtcgtcgccgtagctaa

SEQ ID NO: 21 - protein, 9 aa, Homo sapiens
SYLDSGIHF

SEQ ID NO: 22 - protein, 10 aa, Homo sapiens
ACDPHSGHFV

SEQ ID NO: 23 - protein, 10 aa, Homo sapiens
FIASNGVKLV

SEQ ID NO: 24 - protein, 9 aa, Homo sapiens
AQQITKTEV

SEQ ID NO: 25 - protein, 9 aa, Homo sapiens
AEPINIQTW

SEQ ID NO: 26 - protein, 10 aa, Homo sapiens
GADGVGKSAL

SEQ ID NO: 27 - protein, 9 aa, Homo sapiens
EHEGSGPEL

SEQ ID NO: 28 - protein, 9 aa, Homo sapiens
TYDTVHRHL

SEQ ID NO: 29 - protein, 29 aa, Homo sapiens
EDLTVKIGDFGLATEKSRWSGSHQFEQLS

SEQ ID NO: 30 - protein, 16 aa, Homo sapiens
TMKQICKKEIRRLHQY

SEQ ID NO: 31 - protein, 9 aa, Human papillomavirus type 2
RAHYNIVTF

SEQ ID NO: 32 - protein, 22 aa, Homo sapiens
MDMRVPAQLLGLLLLWLRGARC

SEQ ID NO: 33 - protein, 19 aa, Human papillomavirus HPV16
QAEPDRAHYNIVTFCCKCD

SEQ ID NO: 34 - protein, 19 aa, Homo sapiens
ELFRAAQLANDVVLQIMEL

SEQ ID NO: 35 - protein, 19 aa, Homo sapiens
VHLELASMTNMELMSSIVH

SEQ ID NO: 36 - protein, 18 aa, Gallus gallus
LEQLESIINFEKLTEWTS

SEQ ID NO: 37 - protein, 27 aa, Gallus gallus
AESLKISQAVHAAHAEINEAGREVVGS

SEQ ID NO: 38 - protein, 9 aa, Mus musculus
SAIRSYQYV

SEQ ID NO: 39 - protein, 9 aa, Mus musculus
MALSTYYAL

SEQ ID NO: 40 - protein, 9 aa, Mus musculus
AQLANDVVL

SEQ ID NO: 41 - protein, 9 aa, Mus musculus
ASMTNMELM

SEQ ID NO: 42 - protein, 9 aa, Mus musculus
SNFHFMCAL

SEQ ID NO: 43 - protein, 9 aa, Mus musculus
QIYAFLQGF

SEQ ID NO: 44 - protein, 9 aa, Mus musculus
FALMNLKAL

SEQ ID NO: 45 - protein, 10 aa, Homo sapiens
GVYPMPGTQK

SEQ ID NO: 46 - protein, 9 aa, Homo sapiens
GLLDEDFYA

SEQ ID NO: 47 - protein, 10 aa, Homo sapiens
KINKNPKYKK

SEQ ID NO: 48 - protein, 10 aa, Homo sapiens
SLADEAEVYL

SEQ ID NO: 49 - protein, 10 aa, Homo sapiens
VRTLLSQVNK

SEQ ID NO: 50 - protein, 10 aa, Homo sapiens
RFLEYLPLRF

SEQ ID NO: 51 - protein, 9 aa, Homo sapiens
KLKFVTLVF

SEQ ID NO: 52 - protein, 10 aa, Homo sapiens
DMKARQKALV

SEQ ID NO: 53 - protein, 11 aa, Homo sapiens
WLIRETQPITK

SEQ ID NO: 54 - protein, 9 aa, Homo sapiens
FPKKIQMLA

SEQ ID NO: 55 - protein, 10 aa, Homo sapiens
LRAAFFGKCF

SEQ ID NO: 56 - protein, 9 aa, Homo sapiens
YPVIFKSIM

SEQ ID NO: 57 - protein, 9 aa, Homo sapiens
VTEKLQPTY

SEQ ID NO: 58 - protein, 10 aa, Homo sapiens
WRNILLLSLH

SEQ ID NO: 59 - protein, 9 aa, Homo sapiens
EVLPFFLFF

SEQ ID NO: 60 - protein, 9 aa, Homo sapiens
QTNPVTLQY

SEQ ID NO: 61 - protein, 9 aa, Homo sapiens
TLYSLTLLY

SEQ ID NO: 62 - protein, 9 aa, Homo sapiens
FLIYLDVSV

SEQ ID NO: 63 - protein, 9 aa, Homo sapiens
FFYLLDFTF

SEQ ID NO: 64 - protein, 10 aa, Homo sapiens
IMQTLAGELY

SEQ ID NO: 65 - protein, 10 aa, Homo sapiens
TRSSGSHFVF

SEQ ID NO: 66 - protein, 10 aa, Homo sapiens
VLLGVKLFGV

SEQ ID NO: 67 - protein, 9 aa, Homo sapiens
ALYGFVPVL

SEQ ID NO: 68 - protein, 9 aa, Homo sapiens
ILTGLNYEV

SEQ ID NO: 69 - protein, 9 aa, Homo sapiens
FMPDFDLHL

SEQ ID NO: 70 - protein, 9 aa, Homo sapiens
TESPFEQHI

SEQ ID NO: 71 - protein, 10 aa, Homo sapiens
RLFPGLTIKI

SEQ ID NO: 72 - protein, 9 aa, Homo sapiens
CILGKLFTK

SEQ ID NO: 73 - protein, 10 aa, Homo sapiens
LTDDRLFTCY

SEQ ID NO: 74 - protein, 11 aa, Homo sapiens
GEEDGAGGHSL

SEQ ID NO: 75 - protein, 9 aa, Homo sapiens
GQFLTPNSH

SEQ ID NO: 76 - protein, 9 aa, Homo sapiens
RVSTLRVSL

(19) United States
(12) Patent Application Publication
(10) Pub. No.: US 2024/0139300 A1
(43) Pub. Date: May 2, 2024

(54) mRNA VACCINE COMPOSITIONS AND THEIR USE

(71) Applicant: OSIVAX, Paris (FR)

(72) Inventors: Alexandre LE VERT, Paris (FR); Judith DEL CAMPO, Saint Lary (FR); Fergal HILL, Lyon (FR); Francesco DORO, Lyon (FR)

(21) Appl. No.: 18/471,733

(22) Filed: Sep. 21, 2023

Related U.S. Application Data
(63) Continuation-in-part of application No. PCT/EP2023/057359, filed on Mar. 22, 2023.

(30) Foreign Application Priority Data
Mar. 22, 2022 (EP) 22305341.4
Jun. 7, 2022 (EP) 22305822.3

(51) Int. Cl. A61K 39/00; A61P 35/00

(52) U.S. Cl. CPC A61K 39/0011; A61P 35/00; A61K 2039/53; A61K 2039/6031; A61K 2039/70

(57) ABSTRACT

The invention relates to immunogenic or vaccine compositions and their use in particular in the prevention or treatment of infectious or cancer disorders. More specifically, the immunogenic or vaccine compositions of the present invention comprises a ribonucleic acid (RNA) molecule comprising an open-reading frame encoding a fusion protein, wherein said fusion protein comprises or essentially consists of:
(i) a first polypeptide domain comprising either
   a. an antigen or a fragment thereof comprising at least one epitope of said antigen,
   b. a peptide moiety comprising a single epitope of an antigen, or
   c. a plurality of peptide moieties, wherein each peptide moiety comprises an epitope of an antigen and wherein said peptide moieties are fused together, optionally via peptide linker,
said first polypeptide domain being fused to
(ii) a second polypeptide domain comprising a C4bp-derived oligomerization domain and a positively charged tail.

### CROSS REFERENCE TO RELATED APPLICATIONS

This application is a continuation-in-part application of International Application No. PCT/EP2023/057359, filed on Mar. 22, 2023, which claims priority to European Patent Application No. 22305341.4 filed on Mar. 22, 2022, and European Patent Application No. 22305822.3 filed on Jun. 7, 2022, the contents of each of which are hereby incorporated by references in their entireties.

### REFERENCE TO AN ELECTRONIC SEQUENCE LISTING

The contents of the electronic sequence listing (2023-09-20-OVX-01-PCTN-Sequence Listing.xml; Size: 73,501 bytes; and Date of Creation: Sep. 19, 2023) is herein incorporated by reference in its entirety.

The disclosure relates to mRNA vaccines and their use in particular in the prevention or treatment of infectious or cancer disorders.

### BACKGROUND

mRNA-based vaccination has recently emerged as a powerful technology to prevent infectious diseases such as Covid19 and it is now triggering increasing interest for adaptable, multi-component vaccines [Kumar et al. The mRNA vaccine development landscape for infectious diseases, Nature Reviews Drug Discovery, 2022]. It is also being applied to cancer therapy, specifically in the field of neoepitope-based cancer vaccines: indeed, the versatility of such a technique allows to adapt the mRNA-based vaccines to new emerging viral variants, and to apply it to a personalized neoepitope-based approach for cancer treatment.

Despite all the efforts made in the recent past, there is still a need to further develop novel mRNA vaccines, with improved safety and immunogenicity against pathogen or tumor antigens.

One objective would be to decrease the reactogenicity by reducing the dose of mRNA in particular when formulating multivalent vaccines by reducing the dose of each component.

Another objective would be to increase the cell-based immune response, in particular CD8 T cell response against pathogenic antigens or neo-epitopes, thereby increasing the potency of the vaccine against pathogens and/or tumors.

OVX836 and OVX033 (OSIVAX, Lyon, France) are recombinant proteins under development as a vaccine against influenza and SARS-Cov2 strains respectively. The antigenic part corresponds to the NP sequence of the A/WSN/1933(H1N1) influenza virus or N nucleocapsid of SARS-Cov2 strain, directly fused to OVX313, a sequence derived from the C-terminal oligomerization domain of the human C4b binding protein (hC4BP) [Hofmeyer T, et al. J Mol Biol. 2013; 425:1302-1317, WO200762819, WO201490905].

When fused by deoxyribonucleic acid (DNA) engineering to an antigen, and after protein expression, OVX313 has the unique property to both heptamerize antigens and facilitate the antigen's accessibility to the immune system through Cell-Penetrating features (poly-arginin C-terminus tale), thus increasing their humoral and cellular immune responses [Del Campo J., et al. npj Vaccines. 2019; 4:4].

DNA-based vaccination is another technique using genetic material to be injected in order to allow the production of the antigen of interest directly in the body, for a better and more “natural” presentation to the immune system. DNA-based composition encoding fusion protein with OVX313 were also tested for their immunogenicity [Del Campo J., et al. npj Vaccines. 2019; 4:4; Konrath et al, Cell Reports 2022 “Nucleic acid delivery of immune-focused SARS-CoV-2 nanoparticles drives rapid and potent immunogenicity capable of single-dose protection” ]. Yet, the transposition of a DNA-based vaccine into an mRNA-based platform raises new challenges. mRNA molecules have intrinsic instability issues and structure-related inflammation effects can eventually hamper the correct translation of the antigen of interest. Moreover, the copies of the protein antigen translated per molecule of mRNA is less than the one per molecule of plasmid DNA, making it less available for the processing and the presentation to the immune system. Longer mRNA sequences may also be unstable, leading to poor antigen production in the cells compared to a DNA-based counterpart [Liu M A, Vaccines (Basel). 2019 June; 7(2): 37. Published online 2019 Apr. 24. doi: 10.3390/vaccines7020037].

The inventors now surprisingly found that RNA vaccine compositions encoding fusion proteins with C4bp-derived oligomerization domain provide increased immune response, in particular CD8 or CD4 T cell response, compared to RNA vaccine encoding the same antigenic determinant without a C4bp oligomerization domain, thereby allowing the possibility to reduce the dose amount of RNA and/or more easily providing multivalent RNA vaccine with reduced dosage compared to RNA molecules encoding a full antigen only. The inventors also surprisingly found that RNA vaccine compositions with RNA encoding peptide moiety containing a single T cell epitope can trigger a T cell response (e.g. CD8 T cell response) when fused to C4bp-derived oligomerization domain, thereby allowing the possibility to provide effective T-cell vaccines and mRNA-based anti-cancer immunotherapies.

### BRIEF DESCRIPTION

Thus, in one aspect, the present disclosure relates to an immunogenic composition which comprises a ribonucleic acid (RNA) molecule comprising an open-reading frame encoding a fusion protein, said fusion protein comprising
(i) a first polypeptide domain comprising either
   a. an antigen or a fragment thereof comprising at least one epitope of said antigen,
   b. a peptide moiety comprising a single epitope of an antigen, or
   c. a plurality of peptide moieties, wherein each peptide moiety comprises an epitope of an antigen and wherein said peptide moieties are fused together, optionally via peptide linker,
and wherein said first polypeptide domain is fused to
(ii) a second polypeptide domain comprising a C4bp-derived oligomerization domain and a positively charged tail.

Another aspect of the present disclosure relates to such immunogenic compositions for use in the treatment and/or prophylaxis of an infectious disease or cancer, in a subject in need thereof.

Another aspect of the present disclosure relates to the use of RNA molecule or immunogenic compositions as described herein, in the preparation of a pharmaceutical composition for the treatment or prophylaxis of infectious disease or cancer.

Another aspect of the present disclosure relates to a method of treating or preventing from infectious disease or cancer in a subject in need thereof, said method comprising administering a therapeutically efficient amount of an immunogenic composition as disclosed herein to said subject.

Another aspect of the present disclosure relates to a method of in vivo inducing or increasing a CD8 or CD4 T cell response against a specific antigen in a subject in need thereof (e.g. tumor mutation-derived neoepitope or viral antigens), said method comprising administering a therapeutically efficient amount of an immunogenic composition as disclosed herein to said subject.

### LEGENDS OF THE FIGURES

FIG. 1: IFN-γ-secreting CD8+ T-cell, in the splenocytes stimulated with 5 μg/ml of NP protein or 2 μg/ml of NP₃₆₆₋₃₇₄. Cellular immune responses induced 1 weeks after the last mRNA vaccination were measured in spleen by ELISpots using the immunodominant peptide NP₃₆₆₋₃₇₄ to stimulate CD8+ T cells. The administration CD8+CD4 combination mRNA alone induced no interferon-γ (IFN-γ)-producing CD8+ T cells response (empty dots ◯). On the contrary, we showed that significant amounts of NP₃₆₆₋₃₇₄-specific IFN-γ-producing CD8+ T cells response was induced by CD8+CD4+OVX313 mRNA vaccination (black dots ●), the level of response being significantly increased compared with the response achieved after CD8+CD4 combination mRNA vaccination (p<0.05). Statistics performed using Kruskal-Wallis statistic test followed by the Dunn's multiple comparisons test *p<0.05.

FIG. 2A: IFN-γ-secreting CD8+ T-cell, in the splenocytes stimulated with 5 μg/ml of NP protein or 2 μg/ml of NP₃₆₆₋₃₇₄. Cellular immune responses induced 1 weeks after the last mRNA vaccination were measured in spleen by ELISpots using the immunodominant peptide NP₃₆₆₋₃₇₄ to stimulate CD8+ T cells. The administration CD8 mRNA alone induced no interferon-γ (IFN-γ)-producing CD8+ T cells response in the spleens (empty dots ◯). On the contrary, we showed that significant amounts of NP₃₆₆₋₃₇₄-specific IFN-γ-producing CD8+ T cells response was induced by CD8+OVX313 mRNA vaccination in the spleen (black dots ●), the level of response being significantly increased compared with the response achieved after CD8 mRNA alone vaccination (p<0.05). Statistics performed using Kruskal-Wallis statistic test followed by the Dunn's multiple comparisons test *p<0.05.

FIG. 2B: IFN-γ-secreting CD8+ T-cell, in the lungs stimulated with 5 μg/ml of NP protein or 2 μg/ml of NP₃₆₆₋₃₇₄. The administration of CD8 mRNA alone induced little interferon-γ (IFN-γ)-producing CD8+ T cells response in the lungs (empty dots ◯). On the contrary, we showed that significant amount of NP₃₆₆₋₃₇₄-specific IFN-γ-producing CD8+ T cells response was induced by CD8+OVX313 mRNA vaccination in the lungs (black dots ●), the level of response being significantly increased compared with the response achieved after CD8 mRNA alone vaccination (p<0.05). Statistics performed using Kruskal-Wallis statistic test followed by the Dunn's multiple comparisons test *p<0.05.

FIG. 3A: IFN-γ-secreting CD8+ T-cell, in the splenocytes stimulated with 2 μg/ml of NP₃₆₆₋₃₇₄. Cellular immune responses induced 1 weeks after the last mRNA vaccination were measured in spleen by ELISpots using the immunodominant peptide NP₃₆₆₋₃₇₄ to stimulate CD8+ T cells. The administration CD8-pep+CD4pep-OVX313-tPA mRNA induced higher interferon-γ (IFN-γ)-producing CD8+ T cells response in the spleens (empty black dots) compared to the Buffer (negative control, empty grey dots). On the other hand, we showed that a higher amounts of NP₃₆₆₋₃₇₄-specific IFN-γ-producing CD8+ T cell response was induced by CD8pep+CD4Pep-OVX313 without tPA mRNA vaccination in the spleens (full black dots), the level of response being significantly increased compared to the response achieved after vaccination with CD8-pep+CD4pep-OVX313-tPA mRNA (p<0.05). Statistics performed using Kruskal-Wallis statistic test followed by the Dunn's multiple comparisons test *p<0.05.

FIG. 3B: IFN-γ-secreting CD8+ T-cell, in the lungs stimulated with 2 μg/ml of NP₃₆₆₋₃₇₄. Cellular immune responses induced 1 weeks after the last mRNA vaccination were measured in the lungs by ELISpots using the immunodominant peptide NP₃₆₆₋₃₇₄ to stimulate CD8+ T cells. The administration CD8-pep+CD4pep-OVX313-tPA mRNA induced higher interferon-γ (IFN-γ)-producing CD8+ T cells response in the lungs (empty black dots) compared to the Buffer (negative control, empty grey dots). On the other hand, we showed that a higher amounts of NP₃₆₆₋₃₇₄-specific IFN-γ-producing CD8+ T cell response was induced by CD8pep+CD4Pep-OVX313 without tPA mRNA vaccination in the lungs (full black dots), the level of response being significantly increased compared to the response achieved after vaccination with CD8-pep+CD4pep-OVX313-tPA mRNA (p<0.05). Statistics performed using Kruskal-Wallis statistic test followed by the Dunn's multiple comparisons test *p<0.05.

FIG. 4A: IFN-γ-secreting CD8+ T-cell, in the splenocytes stimulated with 2 μg/ml of NP₃₆₆₋₃₇₄. Cellular immune responses induced 1 weeks after the last mRNA vaccination were measured in spleen by ELISpots using the immunodominant peptide NP₃₆₆₋₃₇₄ to stimulate CD8+ T cells. The administration CD8-Pep₃₆₆₋₃₇₄-OVX313 mRNA induced statistically higher interferon-γ (IFN-γ)-producing CD8+ T cells response in the spleens (black dots) compared to the Buffer (negative control, empty grey triangles) and to its counterpart without OVX313 (CD8-Pep₃₆₆₋₃₇₄-STOP-non-sense mRNA, empty black dots). Statistics performed using Kruskal-Wallis statistic test followed by the Dunn's multiple comparisons test *p<0.05.

FIG. 4B: IFN-γ-secreting CD8+ T-cell, in the lungs stimulated with 2 μg/ml of NP₃₆₆₋₃₇₄. Cellular immune responses induced 1 weeks after the last mRNA vaccination were measured in the lungs by ELISpots using the immunodominant peptide NP₃₆₆₋₃₇₄ to stimulate CD8+ T cells. The administration CD8-Pep₃₆₆₋₃₇₄-OVX313 mRNA induced statistically higher interferon-γ (IFN-γ)-producing CD8+ T cells response in the lungs (black dots) compared to the Buffer (negative control, empty grey triangles) and to its counterpart without OVX313 (CD8-Pep₃₆₆₋₃₇₄-STOP-non-sense mRNA, empty black dots). Statistics performed using Kruskal-Wallis statistic test followed by the Dunn's multiple comparisons test *p<0.05.

FIG. 5: IFN-γ-secreting CD8+ T-cell, in the splenocytes restimulated with 2 μg/ml of HPV16-E7 CD8 epitope.

Cellular immune responses induced 1 weeks after the last mRNA vaccination were measured in the spleens by ELISpots using the immunodominant HPV16E7-CD8 peptide to restimulate CD8+ T cells. The administration HPV16E7-CD8-OVX313-tPA mRNA induced higher interferon-γ (IFN-γ)-producing CD8+ T cells response in the spleens (white squares) compared to the Buffer (negative control, grey dots) and compared to all the other specimens tested, the level of response being significantly increased compared to the response achieved after vaccination with HPV16E7-CD8-nonsense-tPA mRNA (black squares) (p<0.05). Statistics performed using Kruskal-Wallis statistic test followed by the Dunn's multiple comparisons test *p<0.05, **p<0.01.

FIG. 6A: Specific IFNγ secreting cells evaluated by ELISPOT in response to immunization with different vaccine candidates after separate restimulation with both peptides.

The cellular response was evaluated at D27 with an anti-mouse IFNγ ELISPOT.

Cellular immune responses induced 1 weeks after the last mRNA vaccination were measured in the spleens by ELISpots. Splenocytes were prepared and restimulated with CD8 I Reps1.

When encoded into an mRNA, OVX313 significantly increases the cellular immune response to a combination of two CD8 epitopes deriving from MC38 murine colon carcinoma cell line (black dots).

The tPA signal sequence contributed to a statistically better immune response of the OVX313-bearing construct, upon restimulation with one of the two CD8 peptides (derived from Adpgk antigen, black dots—FIG. 6B), when compared to its counterpart without tPA (white dots—FIG. 6B). A similar trend due to tPA was observed upon restimulation with the other peptide (derived from Reps1 antigen, black dots—FIG. 6A), although with a less evident difference between the two constructs with and without tPA.

Statistics performed using Kruskal-Wallis statistic test followed by the Dunn's multiple comparisons test *p<0.05, **p<0.001,***p<0.0001.

FIG. 6B: Specific IFNγ secreting cells evaluated by ELISPOT in response to immunization with different vaccine candidates after separate restimulation with both peptides.

The cellular response was evaluated at D27 with an anti-mouse IFNγ ELISPOT.

Cellular immune responses induced 1 weeks after the last mRNA vaccination were measured in the spleens by ELISpots. Splenocytes were prepared and restimulated with CD8II Adpgk. When encoded into an mRNA, OVX313 significantly increases the cellular immune response to a combination of two CD8 epitopes deriving from MC38 murine colon carcinoma cell line (black dots).

The tPA signal sequence contributed to a statistically better immune response of the OVX313-bearing construct, upon restimulation with one of the two CD8 peptides (derived from Adpgk antigen, black dots—FIG. 6B), when compared to its counterpart without tPA (white dots—FIG. 6B).

Statistics performed using Kruskal-Wallis statistic test followed by the Dunn's multiple comparisons test *p<0.05, **p<0.001,***p<0.0001.

FIG. 7A: FIG. 7A shows tumor growth rate curves.

The addition of OVX313 sequence clearly improves anti-tumor responses, especially when administrated at 0,7 μg/dose (Black squares).

The negative control group, that has received mRNA with an irrelevant Ag (Flu NP epitopes) failed to display any anti-tumor activity (Grey dots).

Statistics performed using Kruskal-Wallis statistic test followed by the Dunn's multiple comparisons test *p<0.05.

FIG. 7B: FIG. 7B shows the final tumor size. The addition of OVX313 sequence clearly improves anti-tumor responses, especially when administrated at 0,7 μg/dose (Black squares).

This construct is highly efficient to trigger anti-tumor responses and subsequent tumor growth reduction, with 4 mice out of 6 remaining tumor-free (black squares).

The OVA CD4-CD8-OVX313-tPA encoding mRNA vaccine is more efficient than the positive control PolyIC OVA vaccine that triggers an efficient anti-tumor immunity, albeit with a less robust tumor growth control and a lower tumor free mice number (2/6) (Black triangles).

The negative control group, that has received mRNA with an irrelevant Ag (Flu NP epitopes) failed to display any anti-tumor activity (Grey dots).

Statistics performed using Kruskal-Wallis statistic test followed by the Dunn's multiple comparisons test *p<0.05.

FIG. 7C: FIG. 7C shows specific IFNγ secreting cells evaluated by ELISPOT in response to immunization with different vaccine candidates after separate restimulation with the CD8 peptide.

Specific IFNγ cellular immune responses induced 5 days after the last mRNA vaccination were measured in the spleens by ELISpots. Splenocytes were prepared and restimulated with the CD8 OVA peptide.

When encoded into an mRNA, OVX313 significantly increases the cellular immune response to a combination of a CD8 and CD4 peptides deriving from OVA in mice implanted with OVA-expressing B16 melanoma tumor (black squares), being this trend in agreement with what observed for tumor growth reduction effect.

Statistics performed using Kruskal-Wallis statistic test followed by the Dunn's multiple comparisons test *p<0.05.

FIG. 7D: FIG. 7D shows specific IFNγ secreting cells evaluating by ELISPOT in response to immunization with different vaccine candidates after separate restimulation with the CD4 peptide.

Specific IFNγ cellular immune responses induced 5 days after the last mRNA vaccination were measured in the spleens by ELISpots. Splenocytes were prepared and restimulated with the CD4 OVA peptides.

When encoded into an mRNA, OVX313 significantly increases the cellular immune response to a combination of a CD8 and CD4 peptides deriving from OVA in mice implanted with OVA-expressing B16 melanoma tumor (black squares), being this trend in agreement with what observed for tumor growth reduction effect.

Statistics performed using Kruskal-Wallis statistic test followed by the Dunn's multiple comparisons test *p<0.05.

FIG. 7E: In FIG. 7E the Pearson correlation between percent specific CD8 T cells secreting IFNg in the spleen at D13 post tumor inoculation and tumor size is shown. A correlation exists between tumor size and specific CD8 cellular response to OVA, where the higher activation of the immune response corresponds to the smaller tumor sizes (****Significance was set at p<0.0001).

FIG. 8A: FIG. 8A shows average tumor growth rate curves. The addition of OVX313 sequence clearly improves anti-tumor responses, both when administrated at 2 μg/dose and at 0.5 μg/dose. The vaccine is highly efficient to trigger anti-tumor responses and subsequent tumor growth reduction, with 6 mice out of 6 remaining tumor-free. The negative control group, that has received mRNA with an irrelevant Ag (Flu NP epitope) failed to display any anti-tumor activity. Specific IFNγ cellular immune responses induced at D=61 after the first mRNA vaccination (24 days after tumor implant) were measured in the spleens by ELISpots. Statistics performed using Kruskal-Wallis statistic test followed by the Dunn's multiple comparisons test *p<0.05.

FIG. 8B: FIG. 8B shows tumor growth rate per group. The addition of OVX313 sequence clearly improves anti-tumor responses, both when administrated at 2 μg/dose and at 0.5 μg/dose. The vaccine is highly efficient to trigger anti-tumor responses and subsequent tumor growth reduction, with 6 mice out of 6 remaining tumor-free. The negative control group, that has received mRNA with an irrelevant Ag (Flu NP epitope) failed to display any anti-tumor activity. Specific IFNγ cellular immune responses induced at D=61 after the first mRNA vaccination (24 days after tumor implant) were measured in the spleens by ELISpots. Statistics performed using Kruskal-Wallis statistic test followed by the Dunn's multiple comparisons test *p<0.05.

FIG. 8C: FIG. 8C shows specific IFNγ secreting cells evaluated by ELISPOT in response to immunization with different vaccine candidates after restimulation with E7 peptide. Splenocytes were prepared and restimulated with the CD8 E7 peptide. When encoded into an mRNA, OVX313 significantly increases the cellular immune response to a CD8 peptide deriving from HPV16-E7 in mice implanted with HPV-E7 expressing TC1 lung tumor cells in a prophylactic approach. From an immunological point of view, when encoded into an mRNA, OVX313 significantly increases the cellular immune response to the E7 CD8 epitope, being this trend in agreement with what observed for tumor growth reduction effect. Statistics performed using Kruskal-Wallis statistic test followed by the Dunn's multiple comparisons test *p<0.05.

FIG. 8D: In FIG. 8D the Pearson correlation between percent specific CD8 T cells secreting IFNg in the spleen at D24 post tumor inoculation and tumor size is shown. A correlation exists between tumor size and specific CD8 cellular response to E7, where the higher activation of the immune response corresponds to the smaller tumor sizes (****Significance was set at p<0.0001).

FIG. 9: Specific IFNγ secreting cells evaluated by ELISPOT in response to immunization with the different vaccine candidates at the two doses, after separate restimulation with the CD8 OVA peptide.

The cellular response was evaluated at D28 with an anti-mouse IFNγ ELISPOT. The constructs containing OVX313 triggered a better activation compared to their counterparts with the STOP-non-sense sequence. A similar difference was observed with the two doses (3 μg and 0.7 μg), although for the OVX313-containing sequence the maximum activation level was achieved already at the lowest dose of 0.7 μg/mouse. Statistical significance was calculated with a Kruskal-Wallis test followed by the Dunn's multiple comparisons test (*p<0.05, **p<0.01, ***p<0.001)

FIG. 10A: IFN-γ-secreting CD8+ T-cell, in the splenocytes restimulated with 2 μg/ml of Flu NP CD8 epitope after DNA vaccination with plasmids encoding one Flu NP-derived CD8 epitope with and without OVX313.

Cellular immune responses induced 1 weeks after the last DNA vaccination were measured in the spleens by ELISpots using the immunodominant NP CD8 peptide to stimulate CD8+ T cells. The administration NP CD8-OVX313 DNA plasmid (black squares) induced higher interferon-γ (IFN-γ)-producing CD8+ T cells response in the spleens compared to the Empty plasmid (negative control—grey dots) and compared to its counterpart without OVX313 (white squares), the level of response being significantly increased compared to the response achieved after vaccination with Flu-CD8-STOP nonsense DNA (p<0.05).

The fold increase of the response between the plasmids bearing the sequences with and without OVX313, can be calculated on the average value obtained for the ELISPOT count per each group and it's equal to 10 in the spleens.

Statistics performed using Kruskal-Wallis statistic test followed by the Dunn's multiple comparisons test (*p>0.05; **p<0.01).

FIG. 10B: IFN-γ-secreting CD8+ T-cell, in the splenocytes restimulated with 2 μg/ml of Flu NP CD8 epitope after DNA vaccination with plasmids encoding one Flu NP-derived CD8 epitope with and without OVX313.

The fold increase of the response between the plasmids bearing the sequences with and without OVX313, can be calculated on the average value obtained for the ELISPOT count per each group and it's equal to 10 in the spleens. The same ration can be calculated for the experiments described in EXAMPLE 2, where the same sequences were compared in an mRNA form, as shown in FIG. 10B, for the spleens. In that case the fold increase was 37 for the spleens, showing a much more powerful effect of the mRNA versions of these constructs. Statistics performed using Kruskal-Wallis statistic test followed by the Dunn's multiple comparisons test (*p>0.05; **p<0.01).

FIG. 10C: IFN-γ-secreting CD8+ T-cell, in the lungs restimulated with 2 μg/ml of Flu NP CD8 epitope after DNA vaccination with plasmids encoding one Flu NP-derived CD8 epitope with and without OVX313.

Cellular immune responses induced 1 weeks after the last DNA vaccination were measured in the lungs by ELISpots using the immunodominant NP CD8 peptide to stimulate CD8+ T cells. The administration NP CD8-OVX313 DNA plasmid (black squares) induced higher interferon-γ (IFN-γ)-producing CD8+ T cells response in the lungs compared to the Empty plasmid (negative control—grey dots) and compared to its counterpart without OVX313 (white squares), the level of response being significantly increased compared to the response achieved after vaccination with Flu-CD8-STOP nonsense DNA (p<0.05).

The fold increase of the response between the plasmids bearing the sequences with and without OVX313, can be calculated on the average value obtained for the ELISPOT count per each group and it's equal to 11 in the lungs.

Statistics performed using Kruskal-Wallis statistic test followed by the Dunn's multiple comparisons test (*p>0.05; **p<0.01).

FIG. 10D: IFN-γ-secreting CD8+ T-cell, in the lungs restimulated with 2 μg/ml of Flu NP CD8 epitope after DNA vaccination with plasmids encoding one Flu NP-derived CD8 epitope with and without OVX313.

The fold increase of the response between the plasmids bearing the sequences with and without OVX313, can be calculated on the average value obtained for the ELISPOT count per each group and it's equal to 11 in the lungs. The same ration can be calculated for the experiments described in EXAMPLE 2, where the same sequences were compared in an mRNA form, as shown in FIG. 10D, for the lungs. In that case the fold increase was 20 for the lungs, showing a much more powerful effect of the mRNA versions of these constructs. Statistics performed using Kruskal-Wallis statistic test followed by the Dunn's multiple comparisons test (*p>0.05; **p<0.01).

### DETAILED DESCRIPTION

### Definitions

In order for the present disclosure to be more readily understood, certain terms are first defined. Additional definitions are set forth throughout the detailed description.

The term “about” as used herein when referring to a measurable value is meant to encompass variations of ±20%; ±10%, ±5% or ±1% from the specified value, as such variations are appropriate to perform the disclosed methods.

As used herein, an “immunogenic composition” refers to a composition which includes at least one antigen, or a nucleic acid encoding at least said antigen, or particles carrying such antigen, which, when injected into a subject, provokes an immune response against such antigen or against a virus, prokaryotic (e.g. bacterial) or eukaryotic (e.g. mammalian tumor cell) cell expressing such antigen.

As used herein, a “vaccine” refers to an immunogenic composition that induces an immune response upon one or more inoculations into a subject. The immune response can be directed against a pathogen (a virus, a bacterium or a parasite) or against tumor cells (expressing tumor associated antigen, or tumor mutation-derived neoepitopes). In some embodiments, the induced immune response provides protective immunity, either to prevent from an infection from a pathogen or to reduce the risk of having severe symptoms of a disease in the context of infectious disease. In some embodiments, the induced immune response is an increased CD8 T cell response against a specific antigen comprising a CD8 T cell epitope. In some embodiments the induced immune response provides a therapeutic effect against a tumor, for example by stopping, delaying or reducing the growth of a tumor in a subject.

As used herein, an “immune response” refers to a process involving the activation and/or induction of an effector function in an immune cell, for example, a T cell, B cell, natural killer (NK) cell, and/or antigen-presenting cell (APC). For example, an immune response includes, without limitation, an antigen-specific activation and/or induction of a helper CD4 T cell or cytotoxic CD8 T cell, an induction of antigen-specific antibody production, activation of APC, macrophage, neutrophil and the like.

As used herein, the term “antigen” or “Ag” refers to a molecule or molecular structure that can bind to a specific antibody or T-cell receptor. The presence of an antigen in the body may trigger an immune response. Antigens can be proteins, peptides or polysaccharides, lipids or nucleic acids. An antigen may comprise several epitopes. The antigen may be natural or non-natural polypeptide, in particular, according to the present disclosure, the antigen is part of a fusion protein, which is expressed by a RNA molecule, for use as an immunogenic composition or a vaccine.

As used herein, the term “epitope”, also called herein as the “antigenic determinant”, is the part of an antigen that is recognized by the immune system, specifically by antibodies, B cells, or T cells (CD4 or CD8 T cells). T-cell epitopes are presented on the surface of antigen-presenting cell, where they are bound to major histocompatibility complex (MHC) molecules. In human, T cell epitopes presented by MHC class I molecule are typically peptides between 8 and 11 amino acids in length, whereas MHC class II molecules present longer peptides, for example between 13-17 amino acids in length. The part of the antigen that is recognized by immunoglobulin or antibodies is called a B-cell epitope.

As used herein, the term “flanking regions” with reference to an epitope refers to the amino acid residues which are naturally found C-terminally and N-terminally to the epitope sequence in the native antigen sequence comprising such epitope, or variant sequences thereof. Preferably, each flanking region refers to a fragment consisting of the 15 or less contiguous amino acids that are directly found C-terminally or N-terminally to the epitope in the native antigen, for example from 2 to 15 contiguous amino acid residues, typically, 2 to 10, or 3 to 8 contiguous amino acid residues, typically 5 contiguous amino acid residues.

The term “amino acid” refers to naturally occurring and unnatural amino acids (also referred to herein as “non-naturally occurring amino acids”), e.g., amino acid analogues and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogues refer to compounds that have the same basic chemical structure as a naturally occurring amino acid, e.g., an alpha carbon that is bound to a hydrogen, a carboxyl group, an amino group, and an R group, e.g., homoserine, norleucine, methionine sulfoxide, methionine methyl sulfonium. Such analogues can have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that function similarly to a naturally occurring amino acid. The terms “amino acid” and “amino acid residue” are used interchangeably throughout.

Substitution refers to the replacement of a naturally occurring amino acid either with another naturally occurring amino acid or with an unnatural amino acid.

As used herein, the term “protein” refers to any organic compounds made of amino acids arranged in one or more linear chains (also referred as “polypeptide”) and folded into a globular form. It includes proteinaceous materials or fusion proteins. The amino acids in such polypeptide chain may be joined together by the peptide bonds between the carboxyl and amino groups of adjacent amino acid residues. The term “protein” further includes, without limitation, peptides, single chain polypeptide or any complex proteins consisting primarily of two or more chains of amino acids. It further includes, without limitation, glycoproteins or other known post-translational modifications. It further includes known natural or artificial chemical modifications of natural proteins, such as without limitation, glycoengineering, pegylation, hesylation, PASylation and the like, incorporation of non-natural amino acids, amino acid modification for chemical conjugation or other molecules, etc . . . .

As used herein, the term “fusion protein” refers to a protein comprising at least one polypeptide chain which is obtained or obtainable by genetic fusion, for example by genetic fusion of at least two nucleic acid fragments encoding functional domains or epitope of distinct proteins. A protein fusion of the present disclosure includes for example one or more peptide moieties consisting of fragments of an antigen and at least one other moiety, the other moiety being a carrier protein comprising a C4bp-derived oligomerization domain and a positively charged tail thereof as described below. A protein fusion in the context of the present disclosure is typically expressed from the coding sequence of an RNA molecule formulated as an immunogenic composition, and administered in a subject in need thereof.

As used herein, the term “fragment” refers to a portion of a protein (in particular a natural protein), e.g. an antigen, that is at least 8 amino acids in length. A fragment of an antigen may be 100% identical to the full length except missing at least one amino acid from the N and/or C-terminal. A fragment may consist of 10% or less, 20% or less, 30% or less, 40% or less, 50% or less, 60% or less, 70% or less, 80% or less, 90% or less, 95% or less of the length of the particular full length native protein, e.g. of a particular full length native antigen.

As used herein, an open reading frame (ORF) refers to a sequence of several nucleotide triplets, which is translated into a peptide, polypeptide, or protein in an appropriate host cell. An open reading frame preferably contains a start codon coding for methionine (ATG or AUG) and is preferably terminated by a stop codon (e.g. TAA, UAA, TAG, UAG, TGA, UGA). An open reading may also be termed “coding sequence”. Unless other specified, an “open reading-frame encoding an amino acid sequence” include all nucleotide sequences that are degenerate versions of each other and that encode the same amino acid sequence.

As used herein, the percent identity between the two sequences is a function of the number of identical positions shared by the sequences (i.e., % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below.

The percent identity between two amino acid sequences can be determined using the Needleman and Wunsch algorithm [Needleman, Saul B. & Wunsch, Christian D. (1970). Journal of Molecular Biology. 48 (3): 443-53].

The percent identity between two nucleotide or amino acid sequences may also be determined using for example algorithms such as EMBOSS Needle (pair wise alignment; available at www.ebi.ac.uk, Rice et al 2000 Trends Genet 16:276-277). For example, EMBOSS Needle may be used with a BLOSUM62 matrix, a “gap open penalty” of 10, a “gap extend penalty” of 0.5, a false “end gap penalty”, an “end gap open penalty” of 10 and an “end gap extend penalty” of 0.5. In general, the “percent identity” is a function of the number of matching positions divided by the number of positions compared and multiplied by 100. For instance, if 6 out of 10 sequence positions are identical between the two compared sequences after alignment, then the identity is 60%. The percent identity is typically determined over the whole length of the query sequence on which the analysis is performed. Two molecules having the same primary amino acid sequence or nucleic acid sequence are identical irrespective of any chemical and/or biological modification.

As used herein, the term “subject” includes any human or nonhuman animal, preferably human. The term “nonhuman animal” preferably includes mammals, such as nonhuman primates, sheep, dogs, cats, horses, etc.

The term “expression vector” refers to a vector comprising a recombinant polynucleotide comprising expression control sequences operatively linked to a nucleotide sequence to be expressed, for example as an in vitro transcribed RNA molecule. Expression vectors include, without limitation, cosmids, plasmids (e.g. naked or contained in liposomes), viruses (e.g. lentiviruses, retroviruses, adenoviruses and adeno-associated viruses) that incorporate the recombinant polynucleotide.

In the context of the present disclosure, conservative substitutions may be defined by substitutions within the classes of amino acids reflected as follows:
Aliphatic residues I, L, V, and M
Cycloalkenyl-associated residues F, H, W, and Y
Hydrophobic residues A, C, F, G, H, I, L, M, R, T, V, W, and Y
Negatively charged residues D and E
Polar residues C, D, E, H, K, N, Q, R, S, and T
Positively charged residues H, K, and R
Small residues A, C, D, G, N, P, S, T, and V
Very small residues A, G, and S
Residues involved in turn A, C, D, E, G, H, K, N, Q, R, S, P, and formation T
Flexible residues Q, T, K, S, G, P, D, E, and R

A “functional variant” is a variant which retains the properties of interest of the native polypeptide.

In preferred embodiments, a variant comprises an amino acid sequence which is at least 50%, 60%, 70%, 80%, 90%, or 95% identical to the native polypeptide sequence.

As used herein, the term “treatment” or “treating” refers to a therapeutic effect that is obtained by suppression, diminution, remission, prevention, or eradication of at least one sign or symptom of a disease.

As used herein, the term “prevent” or “preventing” in relation to a disease refers to one or more of (1) reducing the risk of experiencing or displaying the disease condition or disorder or any symptoms associated to the disease, in an individual who may be exposed at such risk, and (2) reducing the risk of severe disease, or of exhibiting one or more severe symptoms of the disease or (3) reducing the viral shedding, thereby potentially reducing the spread of the virus from one subject to another. In particular, with reference to the prevention of an infectious disease, the term “prevention” may refer to the prevention of infection by the infectious agent, inhibition of the replication of the infectious agent, reduction or elimination of any symptoms associated to the disease, reduction of viral shedding, or reduction of the severity and/or the duration of one or more of the symptoms associated to the infection, or eradication of the infectious agent. As used herein, when referring to reducing the incidence of severe disease or of exhibiting one or more severe symptoms of the disease, the term “protection” may be used interchangeably.

As used herein, the term “coronavirus disease” refers to any disease caused by coronavirus infection, and in particular, SARS (severe acute respiratory syndrome), MERS (middle east respiratory syndrome) or, preferably, COVID-19 (coronavirus disease 2019), and more specifically severe COVID-19.

Prevention or protection is determined in a group of subjects and may not be applicable to all individuals. Typically, prevention or protection may be determined by randomized clinical trials as compared to a control group.

The Fusion Protein

The open reading frame of the RNA molecule for use in the immunogenic composition or vaccine according to the present disclosure encodes a fusion protein which comprises at least the following elements
(i) a first polypeptide domain, comprising the antigenic determinant of the fusion protein,
fused to
(ii) a second polypeptide domain, comprising at least an oligomerization domain for enhancing immunogenicity of the antigenic determinant.

In the present section, the fusion protein is the result of the expression in a host of the RNA molecule according to the present disclosure, for example, as resulting from expression of the RNA molecule in a mammalian subject, after injection of an effective amount of an immunogenic composition of the present disclosure to said subject.

As used herein, the term “fusion protein” refers to an artificial protein wherein at least two distinct polypeptides (hereafter referred as “polypeptide domains”) are linked together by peptide bond and can be generated by translation of an RNA molecule encoding such fusion protein with at least the two distinct polypeptide domains.

Specific embodiments of the first and second polypeptide domains are described in detail in the next sections.

In preferred embodiments, the open reading frame of the RNA molecule encodes a fusion protein wherein the second polypeptide domain is fused C-terminally to the first polypeptide domain, optionally via a peptide linker.

Peptide linkers may be selected from any peptide linker generally used for fusion protein, usually short peptide linkers of 2 to 8 amino acid residues. Preferred peptide linkers, includes glycine-serine linker, such as the dipeptide gly-ser, or gly-ser-ser-ser, or (gly-ser-ser-ser)ₙ, wherein n is an integer between 1 and 4.

In specific embodiments, said fusion proteins form heptameric particles after self-assembling.

In specific embodiments, said fusion proteins form particles with diameters below 100 nm, for example between 5 and 100 nm, and more specifically between 10 and 100 nm, after self-assembling. The diameter of said particle may be measured for example by dynamic light scattering (DLS). DLS measures the hydrodynamic diameter of particles across the size range of approximatively 0.3 nm to 10 μm. DLS measurements are very sensitive to temperature and dispersant viscosity. Therefore, the temperature must be kept constant at 25° C. and the viscosity of the dispersant must be known.

In specific embodiments, the open reading frame of the RNA molecule may further comprise in addition to the coding sequence of the first and second polypeptide domain, the coding sequence of a signal sequence. This signal sequence may facilitate the secretion of the fusion protein, for example once translated in the cells after injection of the immunogenic compositions of the present disclosure or their corresponding RNA encoding molecule.

Hence, in specific embodiments, the fusion protein further includes a signal or secretion peptide sequence. This signal sequence may facilitate the secretion of the fusion protein, for example once translated in the cells after injection of the immunogenic compositions of the present disclosure or their corresponding RNA encoding molecule. Examples of peptide sequence include without limitation the Tissue plasminogen activator (tPA) signal sequence of SEQ ID NO:7, or Human Ig kappa light chain V-Ill region signal peptide of SEQ ID NO:34.

In specific embodiments, the first polypeptide domain of the fusion protein comprises an epitope of a tumor associated antigen or mutation-derived neoepitope and includes a signal or secretion peptide sequence, such as the tPA signal sequence of SEQ ID NO:7.

In other specific embodiments, the fusion protein does not comprise a signal or secretion peptide sequence.

The First Polypeptide Domain Comprising the Antigenic Determinant

According to the present disclosure, the open reading frame of the RNA molecule encodes a first polypeptide domain which comprises either,
a. an antigen or a fragment thereof comprising at least one epitope of said antigen, or
b. a peptide moiety comprising a single epitope of an antigen, or
c. a plurality of peptide moieties, wherein each peptide moiety comprises an epitope of an antigen and wherein said peptide moieties are fused together, optionally via peptide linker,

In a specific embodiment, the first polypeptide domain comprises a native full-length antigen or a fragment of said native full-length antigen comprising one or more epitopes of said native full-length antigen.

As used herein, the term “artificial” refers to a substance which is not found in nature, and for example, which can only be obtained by genetic engineering or DNA recombinant technologies.

As used herein, the term “native” in relation to a polypeptide or antigen, refers to the form of the polypeptide or antigen as occurring in nature, as opposed to “artificial” which refers to modified forms of the polypeptide or antigen, e.g. by genetic engineering and/or DNA recombinant technologies. “Native” antigens include natural mutant versions of antigens, for example cancer mutation-derived neoepitope or viral antigens from natural variant strains.

In other specific embodiments, the first polypeptide domain comprises a peptide moiety comprising a single epitope, preferably T cell epitope, and more preferably CD8 or CD4 T cell epitope, optionally with its flanking regions, thereby forming a first polypeptide domain comprising a single epitope of a specific antigen, for example, a tumor associated antigen or cancer mutation-derived neoepitope.

In other specific embodiments, the first polypeptide domain comprises a plurality of peptide moieties which are directly fused together, or indirectly via peptide linkers, each peptide moiety comprising an epitope of an antigen, optionally with its flanking regions, thereby forming an artificial polypeptide domain comprising different epitopes of one or more antigens fused together. In specific embodiments with a plurality of peptide moieties fused together, each peptide moiety can comprise a distinct epitope of the same antigen, or distinct epitope of different antigens from the same organism or pathogen, or similar epitope of the same antigens from different variant strains of the same organism or pathogen, e.g. different variant strains of a virus.

In other specific embodiments, the first polypeptide domain comprises a plurality of peptide moieties which are directly fused together, or indirectly via peptide linkers, wherein said plurality of peptide moieties may comprise identical peptide moieties (same epitope), distinct epitopes of the same antigen, or epitopes of distinct antigens, or a combination thereof.

In specific embodiments with a plurality of peptide moieties fused together, each peptide moiety can comprise a distinct epitope of the same antigen, or distinct epitope of different antigens from the same tumor, or similar epitopes of the same antigen from different tumor cells, e.g. different mutation-derived neoepitopes from a subject having a cancer disease, or different epitope of antigens from different tumors.

The skilled person will be able to identify the epitope of an antigen for example using web based applications such as SYFPEITHI (http://www.syfpeithi.de/bin/MHCServer.dll/EpitopePrediction.htm, Schuler M M et al. “SYFPEITHI: database for searching and T-cell epitope prediction” Methods Mol Biol. 2007; 409:75-93. doi: 10.1007/978-1-60327-118-9_5) or NetMHCcons—1.1

(https:services.healthtech.dtu.dk/service.php?NetMHCcons-1.1, Karosiene E et al. “NetMHCcons: a consensus method for the major histocompatibility complex class I predictions” Immunogenetics. 2012 March;64(3):177-86. doi: 10.1007/s00251-011-0579-8. Epub 2011 Oct. 20) and corresponding nucleic acid coding sequence for use in the RNA molecule of the present disclosure.

The epitopes of an antigen comprised in the first polypeptide domain may be for example selected among B-cell, CD4 T cell or CD8 T cell epitopes or a combination thereof.

In specific embodiments, said first polypeptide domain comprises from two and up to forty, or more, peptide moieties, either the same or different peptide moieties. In specific embodiments, said first polypeptide domain comprises 1, 2, 3, 4, 5, 6, 7, 8, 9, 10. 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or more, peptide moieties, either the same or different, each peptide moiety comprising a CD8 and/or CD4 T cell epitope of the same antigen or different antigen.

The skilled person will adapt the number and type of epitopes with the desired immunogenic response.

In specific embodiments, said first polypeptide domain comprises two, three, four, five, six, seven, eight, nine, ten or more, different peptide moieties, each peptide moiety comprising a distinct CD8 and/or CD4 T cell epitope of the same antigen or of different antigens.

In a related specific embodiment, at least a first peptide moiety comprises a CD8 T cell epitope of an antigen and a second peptide moiety comprises a CD4 T cell epitope of the same antigen.

In another specific embodiment, at least a first peptide moiety comprises a T cell CD8 epitope of an antigen and a second peptide moiety comprises a second T cell CD8 epitope of the same antigen.

In another specific embodiment, at least a first peptide moiety comprises a CD4 T cell epitope of an antigen.

In specific embodiments, each peptide moiety comprises or essentially consists of an epitope flanked by its corresponding N-terminal and C-terminal flanking regions or variants thereof, preferably wherein each flanking regions consists of 2 to 15, for example of 2 to 10, or of 3 to 8 contiguous amino acid residues, typically 5 amino acid residues.

In related preferred embodiments, each peptide moiety essentially consists of a fragment of an antigen consisting of 8 to 50 contiguous amino acid residues of said antigen with at least one epitope of said antigen, for example a fragment of an antigen of 10 to 30 contiguous amino acid residues, each fragment comprising at least one epitope of said antigen, and wherein said fragments are fused together to form said first polypeptide domain.

In specific embodiments, the antigen or peptide moiety for use in the first polypeptide domain is selected from viral, bacterial, parasitic or tumor antigens.

Preferred examples of viral antigens include without limitation antigens selected from influenza (e.g. hemagglutinin HA2 or nucleoprotein NP), coronavirus (e.g. nucleocapsid N or protein spike S, preferably of SARS-COV2), human papilloma virus (HPV) (e.g. E6 and E7), zika virus (e.g. prM-E, E, NS1, EDIII)), HIV (e.g. gp41 of HIV-1 or HIV-2), Ebola virus (EBOV) (e.g. gp1,2), Hepatitis B virus (e.g. S-antigen), Epstein-Barr virust (EBV) (gH/gL and gB proteins).

Examples of bacterial antigens include, but are not limited to, antigens selected from Neisseria meningitidis (e.g. fHbp, NadA, NHBA), Yersinia enterocolitica (e.gYopB, LcrV) Moraxella catarrhalis (e.g. OMP CD, McaP, PilA1a), Streptococcus pyogenes (e.g. M protein, prgA), Streptococcus agalactiae (e.g. Sip, pilus protein 2a), Pseudomonas Aeruginosa (PiIQ protein).

Examples of parasitic antigens include, but are not limited to, antigens selected from Plasmodium (e.g.PfEMP1, CSP, SSP2), mycobacteria (e.g. antigen 85A, Rv3621c, Rv3618, 38 kDa-ESAT-6), Cryptosporidium (e.g. CP15 antigen) or Leishmania (e.g. leishmania promastigote antigens LaPSA-38S and LiPSA-50S).

Examples of tumor antigens include, but are not limited to, antigens selected from tumor associated antigen, including without limitation, MAGE-C1, MAGE-C2, NY-SEO-1, surviving, 5 T4, MUC1, PSA, PSCA, PSMA, STEAP1, PAP, MAGE-C3, tyrosinase, gp100, CT7, MAGE-A1, MAGE-A3, CEA, or antigens in virus-associated tumors, such as HPV associated tumors, including E7 HPV antigen.

Other examples include tumor mutation-derived neoepitope(s) from beta catenin (CTNNB1) (in melanoma), CDK4 (in melanoma), alpha-actinin-4 (ACTN4) (in non-small cell lung cancer), nuclear transcription factor γ subunit gamma, Acyl-CoA:lysophosphatidylglycerol acyltransferase, KRAS (in Colorectal cancer), Protrudin (ZFYVE27) in pancreatic cancer, calcium-dependent secretion activator 2 (CASPS2) in breast cancer, B-RAF in melanoma, dek-can fusion protein in myeloid leukemia. The antigen may be selected among the natural antigens, for example, natural viral antigens, bacterial antigens or tumor antigens as identified in nature, typically selected among the specific native antigens as disclosed herein, or their antigenic artificial variants thereof.

In specific embodiments, an antigenic artificial variant of an antigen is a fragment of a native antigen having at least 5, 10, 20, 30, 40, 50, 100, 150, 200, 250, 300, 350, 400, 450, 490 consecutive amino acid residues of the corresponding native antigen, for example the viral, bacterial or cancer antigen, typically selected among the specific native antigens as disclosed herein, and comprising at least an epitope of said antigen, typically a CD8 and/or a CD4 T cell epitope, optionally with its flanking regions.

In specific embodiments, an antigenic artificial variant of an antigen is an antigenic polypeptide variant having at least 50%, 60%, 70%, 80%, 90%, 95% or 99% identity to corresponding sequence of the native antigen, typically selected among the specific native antigens as disclosed herein.

In a particular embodiment, said variant differs from the corresponding native antigen, through only amino acid substitutions, with natural or non-natural amino acids, preferably only 1, 2, 3, 4, 5, 6, 7, 8, 9 or 10 amino acid substitutions as compared to the native antigen. In a specific embodiment, a variant is a mutant variant having 1, 2 or 3 amino acid substitutions as compared to the corresponding sequence of the native antigen, typically selected among the specific native antigens as disclosed herein.

In more specific embodiments, the amino acid sequence of said mutant variant may differ from the corresponding sequence of the native antigen through mostly conservative amino acid substitutions; for instance at least 10, such as at least 9, 8, 7, 6, 5, 4, 3, 2 or 1 of the substitutions in the variant are conservative amino acid residue replacements.

More conservative substitutions groupings include: valine-leucine-isoleucine, phenylalanine-tyrosine, lysine-arginine, alanine-valine, and asparagine-glutamine. Conservation in terms of hydropathic/hydrophilic properties and residue weight/size also may be substantially retained in a variant mutant polypeptide as compared to a parent polypeptide of any corresponding natural antigen, typically selected among the specific antigens as disclosed herein.

Embodiments with Influenza Antigens

In specific embodiments, said viral antigen is selected among antigens from influenza, in particular from viral strain of Influenza A, B or C, or combinations thereof. In some embodiments, the strain of Influenza A or Influenza B is associated with birds, pigs, horses, dogs, humans or non-human primates. In some embodiments, the viral strain is selected from the group consisting of H1N1, H3N2, H7N9, and H10N8.

In specific embodiments, the influenza antigen is selected from influenza virus A, more specifically, from strain A/Wilson-Smith/1933 H1N1.

Examples of influenza antigen includes the HA antigen, NA antigen, NP antigen, M1 antigen, M2 ion channel antigen, and any antigenic variants thereof or any combination thereof.

In specific embodiments, the influenza nucleoprotein antigen is the NP antigen of influenza virus A, more specifically, from strain A/Wilson-Smith/1933 H1N1, for example comprising the polypeptide of SEQ ID NO:8, or their corresponding antigenic variants, preferably having at least 90% identity to SEQ ID NO:8, more preferably at least 95%, 96%, 97%, 98%, or at least 99% identity to SEQ ID NO:8.

In specific embodiments, a mutant variant comprises a polypeptide which is identical to SEQ ID NO:8, except for 1, 2 or 3 amino acid residues which have been replaced by another natural amino acid by conservative amino acid substitutions as defined above.

In specific embodiments, said first polypeptide domain comprises a plurality of peptide moieties or a fragment of an antigen, each peptide moiety or fragment of an antigen comprising an epitope selected among the peptide of SEQ ID NO:9 and/or SEQ ID NO:10, or their antigenic variants.

In specific embodiments, said first polypeptide domain comprises at least two peptide moieties, a first peptide moiety comprising an epitope of SEQ ID NO:9 and a second peptide moiety comprising an epitope of SEQ ID NO:10, or their antigenic variants.

In specific embodiments, said first polypeptide domain comprises at least two peptide moieties, a first peptide moiety comprising or essentially consisting of a first fragment of NP antigen of SEQ ID NO: 11 and a second peptide moiety comprising or essentially consisting of a second fragment of NP antigen SEQ ID NO12., or their antigenic variants.

Embodiments with Coronavirus Antigens

In specific embodiments, said viral antigen is selected among antigens from coronavirus, in particular, SARS-Cov1, MERS-Cov and SARS-Cov2, and more specifically derived from viral strain of SARS-Cov2 selected from the group consisting of Wuhan (A), Europe (B.1), Alpha—UK (B.1.1.7), Beta S. Africa (B.1.351), Gamma Brazil (P.1), Delta India (B.1.617.2) and Omicron strain.

Examples of coronavirus antigen includes the natural Spike S antigen or the nucleocapsid antigen, Membrane protein M, Envelope Protein (Protein E) and any antigenic variants thereof.

In specific embodiments, the antigen is the nucleocapsid N antigen of SARS-Cov2 strain, more specifically, from strain Wuhan (A), for example comprising the polypeptide of SEQ ID NO:1 or their corresponding variant selected from the group consisting of Europe (B.1), Alpha—UK (B.1.1.7), Beta S. Africa (B.1.351), Gamma Brazil (P.1), Delta India (B.1.617.2) and Omicron strain, or their antigenic variants.

In specific embodiments, said first polypeptide domain comprises or essentially consists of the Receptor Binding Domain (RBD) of the protein spike from coronavirus strain, for example from SARS-Cov2 strain, more specifically derived from viral strain of SARS-Cov2 selected from the group consisting of Wuhan (A), Europe (B.1), Alpha—UK (B.1.1.7), Beta S. Africa (B.1.351), Gamma Brazil (P.1), Delta India (B.1.617.2) and Omicron strain.

Embodiments with Tumor Antigens

An RNA encoding a fusion protein as described herein wherein the first polypeptide domain comprise at least a tumor antigen of a fragment thereof, or at least an epitope of a tumor, should be capable of inducing a T cell response against said tumor antigen, e.g. CD8 T cell response and/or stimulating an anti-tumor response against a tumor expressing said antigen.

In specific embodiments, the RNA molecule comprises at least one coding sequence of a peptide moiety comprising at least an epitope, optionally with its flanking region, e.g., a T cell epitope, preferably a CD8 T or CD4 T cell epitope, of a tumor antigen as defined herein, which antigen is derived or identified from a tumor cell, preferably a mammalian, more preferably human, tumor cell. In specific embodiments, the RNA molecule comprises a coding sequence of a plurality of peptide moieties (for example from 2 to 40 peptide moieties), each peptide moiety encoding at least an epitope of a tumor antigen as defined herein, optionally with its flanking regions, preferably at least one or more CD8 T cell epitope(s). The plurality of peptide moieties may include a plurality of the same peptide moieties (same epitopes) or distinct epitopes of the same antigen or distinct epitopes of distinct antigens, or a combination thereof.

Any protein produced in a tumor cell that has an abnormal sequence or structure due to mutation can act as a tumor antigen. Mutation of protooncogenes and tumor suppressors which lead to abnormal protein production are the cause of the tumor and thus such abnormal proteins are called tumor-specific antigens. Examples of tumor-specific antigens include the abnormal products of ras and p53 genes. In contrast, mutation of other genes unrelated to the tumor formation may lead to synthesis of abnormal proteins which are called tumor-associated antigens.

A tumor antigen is preferably located in or on the surface of a tumor cell derived from a mammalian; preferably from a human tumor, such as systemic, or a solid tumor.

In specific embodiments said antigen is selected from tumor associated antigen or tumor mutation-derived neoepitope(s).

Examples of tumor associated antigen include without limitation, MAGE-C1, MAGE-C2, NY-SEO-1, surviving, 5 T4, MUC1, PSA, PSCA, PSMA, STEAP1, PAP, MAGE-C3, tyrosinase, gp100, CT7, MAGE-A1, MAGE-A3, CEA, or antigens in virus-associated tumors, such as HPV associated tumors, including E7 HPV antigen.

Other examples include tumor mutation-derived neoepitope(s) from beta catenin (CTNNB1) (in melanoma), CDK4 (in melanoma), POLA2 (in melanoma), CO18A1 (in melanoma), GANAB (in melanoma), NSDHL (in melanoma), AHNAK (in melanoma), FAM3C (in melanoma), KIFC2 (in melanoma), CDK12 (in melanoma), PLEKHM2 (in melanoma), RECQL5 (in melanoma), TFDP2 (in melanoma), GNB5 (in melanoma), XPNPEP1 (in melanoma), UGGT2 (in melanoma), GAS7 (in melanoma), HELZ2 (in melanoma), CENPL (in melanoma), WDR46 (in melanoma), PRDX3 (in melanoma), GCN1L1 (in melanoma), NCAPH2 (in melanoma), MYO1B (in melanoma), CIT (in melanoma), DCAK2 (in melanoma), ACPP (in melanoma), FAM50B (in melanoma), COL22A1 (in melanoma), DDX3X (in melanoma), VPS16 (in melanoma), TDX4 (in melanoma), ITGA9 (in melanoma), CASP1 (in melanoma), AFMID (in melanoma), alpha-actinin-4 (ACTN4) (in non-small cell lung cancer), nuclear transcription factor γ subunit gamma, Acyl-CoA:lysophosphatidylglycerol acyltransferase, KRAS (in Colorectal cancer), Protrudin (ZFYVE27) in pancreatic cancer, calcium-dependent secretion activator 2 (CASPS2) in breast cancer, B-RAF in melanoma, dek-can fusion protein in myeloid leukemia.

More specifically, tumor mutation-derived neoepitope(s) may be selected from one or more of the following sequences:

(SEQ ID NO: 21)
SYLDSGIHF from beta catenin (CTNNB1) (in melanoma);
(SEQ ID NO: 22)
ACDPHSGHFV from CDK4 (in melanoma);
(SEQ ID NO: 65)
TRSSGSHFVF from POLA2 gene of human melanoma,
(SEQ ID NO: 66
VLLGVKLFGV from COL18A1 gene of human melanoma,
(SEQ ID NO: 67)
ALYGFVPVL from GANAB gene of human melanoma,
(SEQ ID NO: 68)
ILTGLNYEV from NSDHL gene of human melanoma,
(SEQ ID NO: 69)
FMPDFDLHL from AHNAK gene of human melanoma
(SEQ ID NO: 70)
TESPFEQHI from FAM3C gene of human melanoma,
(SEQ ID NO: 71)
RLFPGLTIKI from KIF2C gene of human melanoma,
(SEQ ID NO: 72)
CILGKLFTK from CDK12 gene of human melanoma,
(SEQ ID NO: 73)
LTDDRLFTCY from PLEKHM2 gene of human melanoma,
(SEQ ID NO: 74)
GEEDGAGGHSL from RECQL5 gene of human melanoma,
(SEQ ID NO: 75)
GQFLTPNSH from TFDP2 gene of human melanoma,
(SEQ ID NO: 76)
RVSTLRVSL from GNB5 gene of human melanoma,
(SEQ ID NO: 53)
WLIRETQPITK from XPNPEP1 gene of human melanoma,
(SEQ ID NO: 46)
GLLDEDFYA from UGGT2 gene of human melanoma,
(SEQ ID NO: 48)
SLADEAEVYL from GAS7 gene of human melanoma,
(SEQ ID NO: 60)
QTNPVTLQY from the HELZ2 gene of human melanoma,
(SEQ ID NO: 61)
TLYSLTLLY from the CENPL gene of human melanoma,
(SEQ ID NO: 62)
FLIYLDVSV from the WDR46 gene of human melanoma,
(SEQ ID NO: 63)
FFYLLDFTF from the PRDX3 gene of human melanoma,
(SEQ ID NO: 64)
IMQTLAGELY from the GCN1L1 of human melanoma,
(SEQ ID NO: 45)
GVYPMPGTQK from NCAPH2 gene of human melanoma,
(SEQ ID NO: 47)
KINKNPKYKK from MYO1B gene of human melanoma,
(SEQ ID NO: 49)
VRTLLSQVNK from CIT gene of human melanoma,
(SEQ ID NO: 50)
RFLEYLPLRF from DCAKD gene of human melanoma,
(SEQ ID NO: 51)
KLKFVTLVF from ACPP gene of human melanoma,
(SEQ ID NO: 52)
DMKARQKALV from FAM50B gene of human melanoma,
(SEQ ID NO: 53)
LPNEYAFVTT from COL22A1 gene of human melanoma,
(SEQ ID NO: 54)
FPKKIQMLA from DDX3X gene of human melanoma,
(SEQ ID NO: 55)
LRAAFFGKCF from VPS16 gene of human melanoma,
(SEQ ID NO: 56)
YPVIFKSIM from TBX4 gene of human melanoma,
(SEQ ID NO: 57)
VTEKLQPTY from ITGA9 gene of human melanoma,
(SEQ ID NO: 58)
WRNILLLSLH from CASP1 gene of human melanoma,
(SEQ ID NO: 59)
EVLPFFLFF from AFMID gene of human melanoma;
(SEQ ID NO: 23)
FIASNGVKLV from alpha-actinin-4 (ACTN4) (in non-small cell lung cancer);
(SEQ ID NO: 24)
AQQITKTEV from nuclear transcription factor Y subunit gamma (in non-small
cell lung cancer),
(SEQ ID NO: 25)
AEPINIQTW from acyl-CoA:lysophosphatidylglycerol acyltransferase (in
bladder cancer),
(SEQ ID NO: 26)
GADGVGKSAL from KRAS (in Colorectal cancer),
(SEQ ID NO: 27)
EHEGSGPEL from Protrudin (ZFYVE27) (in pancreatic cancer),
(SEQ ID NO: 28)
TYDTVHRHL from calcium-dependent secretion activator 2 (CASPS2) (in
breast cancer),
(SEQ ID NO: 29)
EDLTVKIGDFGLATEKSRWSGSHQFEQLS from B-RAF in melanoma,
(SEQ ID NO: 30)
TMKQICKKEIRRLHQY from dek-can fusion protein in myeloid leukemia.

Accordingly, in specific embodiments, the immunogenic compositions comprise an RNA molecule encoding said fusion protein, wherein said fusion protein comprises one or more tumor mutation-derived neoepitope(s) or tumor associated antigen or an antigenic fragment or variant thereof, for example as expressed in a tumor of a subject in need of said immunogenic composition.

The Second Polypeptide Domain Comprising a C4bp-Derived Oligomerization Domain and a Positively Charged Tail

The open-reading frame of the RNA molecule for use in the immunogenic composition or vaccine according to the present disclosure encodes a second polypeptide domain to which the first polypeptide domain is fused, thereby rendering the resulting fusion protein more immunogenic, when expressed in a subject.

The function of the second polypeptide domain is therefore to increase the immunogenicity of the antigenic determinant to which it is fused.

According to the present disclosure, the second polypeptide domain for use in the fusion protein comprises a C4bp-derived oligomerization domain and a positively charged tail.

The complement inhibitor C4-binding protein (C4bp) is an abundant plasma protein first discovered in mice. Its natural function is to inhibit the classical and lectin pathways of complement activation. The last exon of the C4bp alpha chain gene encodes the only domain in the protein which does not belong to the complement control protein family. This non-complement control protein domain contains 57 amino acid residues in human and 54 amino acid residues in mice and is both necessary and sufficient for the oligomerization of the C4bp. It has been found that, when fused to antigens, said C4bp oligomerization domain is also necessary and sufficient for the oligomerization of the resulting fusion protein.

In specific embodiments, said C4bp-derived oligomerization domain enables the fusion protein to form a heptameric protein after self-assembling.

PCT/IB2004/002717 and PCT/EP03/08926 describe the use of mammalian C4bp-derived oligomerization domains to increase the immunogenicity of antigens in mammals. WO2007/062819 further describe a C4bp-derived oligomerization domain of chicken species and variants thereof.

In preferred embodiments, in order to minimize self-immune reaction, the C4bp-derived oligomerization domain has an identity to human C4bp oligomerization domain which is lower than 30%, preferably lower than 20%.

In particular, in specific embodiments, said C4bp-derived oligomerization domain comprises or essentially consists of SEQ ID NO:2.

In specific embodiments, a functional variant of the C4bp-derived oligomerization domain has at least 50%, 60%, 70%, 80%, 90%, 95% or 99% identity to SEQ ID NO:2.

A functional variant may include any variant with one or more amino acid additions, deletions and/or substitutions as compared to SEQ ID NO:2 which retain the oligomerization property of the polypeptide of SEQ ID NO:2.

In a particular embodiment, said variant differs from SEQ ID NO:2, through only amino acid substitutions, with natural or non-natural amino acids, preferably only 1, 2, 3, 4, 5, 6, 7, 8, 9 or 10 amino acid substitutions with natural amino acids. In a specific embodiment, a variant is a mutant variant having 1, 2 or 3 amino acid substitutions with natural amino acids as compared to SEQ ID NO:2.

In more specific embodiments, the amino acid sequence of said mutant variant may differ from the self-assembling polypeptide of SEQ ID NO:2 through mostly conservative amino acid substitutions; for instance, at least 10, such as at least 9, 8, 7, 6, 5, 4, 3, 2 or 1 of the substitutions in the variant are conservative amino acid residue replacements.

The carrier protein further comprises a C-terminal tail consisting of positively charged peptide. The C-terminal tail is preferably a peptide consisting of 6-10 amino acids, with at least 50% of positively charged amino acids. Amino acids with positive charges include arginine or lysine. Examples of such positively charged peptide are disclosed in WO2014/090905 and WO2014/147087.

In preferred embodiments, said positively charged tail comprises the sequence ZXBBBBZ (SEQ ID NO:3), wherein (i) Z is absent or is any amino acid, (ii) X is any amino acid, and (iii) B is an arginine or a lysine, preferably said positively charged tail comprises or essentially consists of the sequence of SEQ ID NO:4.

In more preferred embodiments, said second polypeptide domain essentially consists of OVX313 polypeptide, corresponding to the polypeptide of SEQ ID NO:5.

In specific embodiments, said carrier protein is a functional variant of OVX313 polypeptide of SEQ ID NO:5 having at least 70%, 80%, or more preferably at least 90% identity to SEQ ID NO:5.

In other embodiments, said second polypeptide domain is a functional variant of OVX313 polypeptide of SEQ ID NO:5 which differ from SEQ ID NO:5, by only 1, 2, 3, 4, 5, 6, 7, 8, 9, or 10 amino acids by amino acid substitution. In other embodiments, said carrier protein is a functional variant of OVX313 polypeptide of SEQ ID NO:5 which differ from SEQ ID NO:5, by only 1, 2, 3, 4, 5, 6, 7, 8, 9, or 10 amino acids by conservative amino acid substitution.

Specific Embodiments of the Open Reading Frame Encoding the Fusion Protein

The RNA molecule for use in the immunogenic composition or vaccine includes an open reading frame comprising the coding sequences for any of the first polypeptide domain fused to any of the second polypeptide domain as described in the previous sections, optionally with a signal sequence.

In specific embodiments, said open reading frame encodes a fusion protein comprising the influenza nucleoprotein antigen of influenza virus A, more specifically, from strain A/Wilson-Smith/1933 H1N1, said open reading frame having a coding sequence of the first polypeptide domain, said coding sequence having at least 90% identity to SEQ ID NO:13, more preferably at least 95%, 96%, 97%, 98%, or at least 99% identity, or 100% identity to SEQ ID NO:13.

In specific embodiments, said open reading frame encodes a fusion protein wherein said first polypeptide domain comprises a plurality of peptide moieties or fragments of an antigen, each peptide moiety or fragment, comprises a coding sequence of an epitope selected from SEQ ID NO:9 and SEQ ID NO:10, or variant nucleotide sequence encoding antigenic variants of said epitope with at least 90% identity, more preferably at least 95%, 96%, 97%, 98%, or at least 99% identity to the corresponding SEQ ID NO:9 or SEQ ID NO:10.

In specific embodiments, said open reading frame encodes a fusion protein wherein said first polypeptide domain comprises at least two peptide moieties, a first peptide moiety comprising an epitope encoded by SEQ ID NO:14 and a second peptide moiety comprising an epitope encoded by SEQ ID NO:15, or variant nucleotide sequences encoding antigenic variants with at least 90% identity, more preferably at least 95%, 96%, 97%, 98%, or at least 99% identity to the corresponding SEQ ID NO:14 or SEQ ID NO:15.

In specific embodiments, said open reading frame encodes a fusion protein wherein said first polypeptide domain comprises at least two peptide moieties, a first peptide moiety comprising or essentially consisting of a first fragment of NP antigen, as encoded SEQ ID NO:16, and a second fragment of NP antigen, as encoded by SEQ ID NO:17, or variant nucleotide sequences encoding antigenic variants with at least 90% identity, more preferably at least 95%, 96%, 97%, 98%, or at least 99% identity to the corresponding SEQ ID NO:16 or SEQ ID NO:17.

In preferred embodiments, said open reading frame encodes a fusion protein of CD4 and CD8 epitopes of the influenza nucleocapsid NP antigen, said fusion protein comprising or essentially consisting of SEQ ID NO:18, for example encoded by a coding sequence comprising SEQ ID NO19, or variant nucleotide sequences encoding antigenic variants with at least 90% identity, more preferably at least 95%, 96%, 97%, 98%, or at least 99% identity to the corresponding SEQ ID NO:18.

In preferred embodiments, said open reading frame encodes a fusion protein which essentially consists of OVX033 polypeptide, corresponding to the polypeptide of SEQ ID NO:6, and encoded by SEQ ID NO:20.

The RNA Molecule for Use in the Immunogenic Composition or Vaccine

As used herein, the term “ribonucleic acid” or “RNA” or “mRNA” refers to a nucleic acid molecule which is a polymer of nucleotides, these nucleotides being usually adenosine-monophosphate, uridine-monophosphate, guanosine monophosphate and cytidine monophosphate which are connected to each other along a backbone, formed by phosphodiester bonds between a sugar (ribose) of a first and a phosphate moiety of a second, adjacent monomer. Usually, messenger RNA may be obtainable by transcription of a DNA sequence, e.g. inside a cell. In eukaryotic cells, transcription is performed inside the nucleus and results in a premature RNA which is then processed in a messenger RNA (abbreviated as mRNA). Processing of premature RNA generally comprises a variety of different posttranscriptional modifications such as splicing, 5′-capping, polyadenylation, export from the nucleus or the mitochondria. Accordingly, a mature mRNA typically comprises a 5′-cap, a 5-untranslated region (UTR), an open reading frame (ORF), a 3′ UTR, and a poly(A) sequence.

As used herein, the term “RNA” or “mRNA”, in reference to the immunogenic compositions or vaccines of the present disclosure, preferably refers to a synthetic RNA, including chemically modified RNA molecules, in particular for stabilization of the RNA molecule, typically to render them more stable to disintegration or degradation, e.g., by environmental factors or enzymatic digest. For example, the RNA is a “nucleoside-modified” nucleic acid comprising at least one modified nucleoside that are capable of being translated by translational machinery in a cell. Exemplary modified nucleosides are described for example in Pardi N at al. Nature Reviews Drug Discovery volume 17, pages 261-279 (2018); Karikó K et al., Mol Ther. 2008 November; 16(11):1833-40; and, Andries O et al. J Control Release. 2015 Nov. 10; 217( ):337-44. For example, an mRNA may be modified by replacement of some or all of the uridines with pseudouridine, 1-methyl pseudouridine or other modified uridine. In other embodiments, the mRNA may include some or all cytidines replaced by methylated cytidines.

The RNA molecule for use in the immunogenic composition should direct the expression of the fusion protein and generate an immune response against the antigen or epitope(s) carried by the fusion protein.

In specific embodiments, the RNA molecule is a mRNA comprising at least, from 5′ to 3′,
(i) a 5′-cap,
(ii) a 5-untranslated region (UTR),
(iii) an open reading frame (ORF) including coding sequences as described in the previous sections and, optionally intron (non-coding) sequences,
(iv) a 3′ UTR, and
(v) a poly(A) sequence.

For example, the 5′ cap structure may contain a 7-methylguanosine nucleoside linked through a triphosphate bridge to the 5′ end of the mRNA, thereby protecting from degradation by exonuclases. 5′cap1 structure may be generated using Vaccinia capping enzyme and 2′-O-methyltransferase enzymes (CellScript, Madison, WI). It also acts synergistically with the poly(A) tail at the 3′ end, poly(A) binding proteins and translation initiation factor proteins to circularize mRNA and recruit ribosomes for initiating translation. The first or second nucleotide from the 5′ end may also be methylated on the 2′ hydroxyl of the ribose (2′-O-methylation) for preventing recognition by cytosolic sensors of viral RNA, and hence prevents unintended responses.

The poly(A) may for example have a length of 100-400 nucleotides, for example 100-150 nucleotides to interact with poly(A) binding proteins and form complexes for initiating translation. ATP analogs may be incorporated into the poly(A) tail using poly(A) polymerase. ATP analogs can further increase the stability of the RNA.

In specific embodiments, the 5′UTR can contain the Kozak sequence of the endogenous gene or contain a 5′UTR that is not endogenous to the gene, for example a consensus Kozak sequence. Such consensus Kozak sequence can increase the efficiency of translation of some RNA transcripts.

In specific embodiments, naturally occurring UTRs (5′ and 3′) flanking the open reading frame of highly expressed genes, such as the α and β-globin gens, may be preferred for synthetic mRNAs. Alternative sequences may be used, including optimized sequences depending on the desired application and intended cell target. Engineered UTR sequences may minimize mRNA degradation by excluding miRNA-binding sites and AU-rich regions in the 3′UTR. They also may minimize regions that prevent ribosomes from scanning the mRNA transcript, such as sequences with secondary and tertiary structures (for example, hairpins) in the 5′ UTR.

In specific embodiments, the coding sequence of the open reading frame of the RNA molecule may be optimized to increase translation without altering the protein sequence for example by replacing rarely used codons with more frequently occurring codons that encode the same amino acids. Accordingly, in certain embodiments, the RNA molecule for use in the immunogenic composition or vaccine of the disclosure includes optimized coding sequences and not the corresponding natural coding sequence of an antigen. For example, in specific embodiments, when it is possible without altering the amino acid sequence, RNA codons have G or C instead or A or U at the third position of the open reading frame.

In specific embodiments, the RNA molecule incorporates modified nucleosides (hereafter referred as “nucleoside-modified RNA”), such as pseudouridine, N1-methylpseudouridine or other nucleoside analogues, in particular to prevent recognition by pattern recognition receptors, such as TLR3, TLR7 or TLR8 receptors. Methods for preparing nucleoside-modified RNA for use in RNA immunogenic composition or mRNA vaccines are disclosed for example in Pardi N at al. Nature Reviews Drug Discovery volume 17, pages 261-279 (2018); Karikó K et al., Mol Ther. 2008 November; 16(11):1833-40; Andries O et al. J Control Release. 2015 Nov. 10; 217( ):337-44; Pardi et al. “In Vitro Transcription of Long RNA Containing Modified Nucleosides” Methods Mol Biol. 2013; 969:29-42. doi: 10.1007/978-1-62703-260-5_2. Accordingly, in preferred embodiments, the RNA molecule of the disclosure is a nucleoside-modified RNA.

Methods for Preparing the RNA Molecule

The RNA molecule for use according to the present disclosure may be prepared by any conventional methods for preparing RNA molecules for use in RNA immunogenic compositions or RNA vaccines. For example, the RNA molecule may be in vitro transcribed to mimic the structure of the natural RNA while improving its stability and increasing translation. Hence, in specific embodiments, the immunogenic or vaccine compositions of the present disclosure include in vitro transcribed (IVT) RNA encoding the above-described fusion protein.

Typically, synthetic mRNA may be transcribed in vitro from a corresponding DNA plasmid or synthetic DNA, phage DNA or cDNA, for example, by using the bacteriophage RNA polymerase, e.g. T7, T3 or SP6 RNA polymerase. It may be co-transcriptionally capped (for example using CleanCap, as developed by TriLink BioTechnologies) with a 2′-O-methylated cap and is preferably purified to remove double-stranded RNA (dsRNA), contaminants, reactants, and incomplete transcripts.

In addition, it is possible to introduce a short UGC linker in the poly(A) tail, in particular to overcome stability issues during production with long poly(A) tail, for example >100 bp.

The present disclosure also pertains to the expression vector for in vitro transcription of said RNA molecule as described herein, such as DNA plasmid encoding such RNA molecule.

The present disclosure also pertains to a composition comprising a purified synthetic RNA molecule, devoid of dsRNA, contaminants or incomplete transcripts, for example as obtained from in vitro transcription and purification.

The present disclosure further relates to a method for preparing said RNA molecule as disclosed herein, said method comprising;
(i) providing an expression vector encoding said RNA molecule, e.g., a DNA plasmid,
(ii) in vitro transcribing said RNA molecule from said expression vector, and,
(iii) purifying said RNA molecule.

Methods of preparing said RNA molecule are for example described in Sousa Rosa S. et al. “mRNA vaccines manufacturing: Challenges and bottlenecks” Vaccine. 2021 Apr. 15; 39(16): 2190-2200.

Immunogenic Compositions

In another aspect, the present disclosure provides a composition, e.g. an immunogenic composition containing an RNA molecule as described in the previous sections, and one or more pharmaceutically acceptable excipients.

In specific embodiments, the composition comprises suitable delivery vehicles for in vivo delivery. In particular, because mRNA is large and negatively charged, it cannot pass through the anionic lipid bilayer of cell membrane. Moreover, inside the body, it is engulfed by cells of the innate immune system and degraded by nucleases. In vivo delivery requires vehicles that can transfect cells, without causing toxicity or unwanted immunogenicity.

In specific embodiments, non-viral delivery system is used for delivery of the mRNA molecule. The mRNA molecule may be associated with lipids, for example, as liposomes, wherein the mRNA molecule is encapsulated, entrapped or complexed.

A suitable vehicle for in vivo delivery of mRNA molecule is nanoparticle encapsulating mRNA, typically selected from lipid-based nanoparticles (LNP), polyplexes and polymeric nanoparticles.

Hence, in specific embodiments, the immunogenic composition or RNA molecule is formulated in a lipid-based nanoparticles, polyplexes or polymeric nanoparticles, preferably lipid-based nanoparticles (LNP).

In specific embodiments, the lipid nanoparticles may have a mean diameter of from about 30 nm to about 150 nm. In some embodiments, the lipid nanoparticles may comprise any lipid capable of forming a particle to which the one or more mRNA molecules are attached, or in which the one or more mRNA molecules are encapsulated. It may comprise one or more cationic lipids and one or more stabilizing lipids, including neutral or pegylated lipids.

In specific embodiments, lipid-based nanoparticles include one or more of the following components:
(i) an ionizable lipid,
(ii) cholesterol, or cholesterol analogues, for enhancing the stability of the nanoparticles by filling gaps between lipids,
(iii) a helper phospholipid, for modulating nanoparticle fluidity and enhance efficacy
(iv) a PEGylated lipid, to stabilize the LNP, regulate nanoparticle size by limiting lipid fusion,
which alone or as combined together encapsulate the mRNA core.

Examples of lipid-based nanoparticles include cationic lipids, such as DOTMA and their synthetic analogues DOTAP, ionizable lipids formulated with mRNA into nanoparticles in acidic buffer, such as DODAP and DODMA, DLinDMA and DLin-MC3-DMA, or other potent lipids such as C12-200, 503O₁₃, 3060, SM-102 and ALC-0315. Lipid-based nanoparticles may also include lipids containing polycyclic adamantane tails, targeting T cells in vivo, such as 11-A-M or those containing cyclic imidazole heads, such as 93-O17S. For a review, please see Hou et al 2021 Nature Reviews/Materials Vol 6, 1078-1094.

Examples of helper (or neutral) lipids, includes for example distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoyl-phosphatidyethanol amine (SOPE), and 1,2-dielaidoyl-sn-glycero-3-phophoethanolamine (transDOPE). In one embodiment, the neutral lipid is 1,2-distearoyl-sn-glycero-3-phosphocholine (DSPC). In some embodiments, the LNPs comprise a neutral lipid selected from DSPC, DPPC, DMPC, DOPC, POPC, DOPE and SM.

In specific embodiments, the LNPs further comprise a steroid or steroid analogue.

The PEGylated lipid components of LNP consists of polyethylene glycol (PEG) conjugated to an anchoring lipid such as DPME or DMG. Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols. The molecular weight of PEG can vary from 350 to 3,000 Da, for example between 1,500 and 2,500 Da, and the lipid anchor's tail length can vary from 10 to 18 carbons, for example between 12 and 15 carbon-low saturated lipid anchors.

Other formulations of lipid nanoparticles are reviewed in Hou, X., Zaks, T., Langer, R. et al. Lipid nanoparticles for mRNA delivery. Nat Rev Mater 6, 1078-1094 (2021); and Reichmuth A M et al. “mRNA vaccine delivery using lipid nanoparticles” Ther Deliv. 2016 May; 7(5): 319-334.

In other specific embodiments, the immunogenic composition or RNA molecule is formulated in polyplexes or polymeric nanoparticles. Cationic polymers that condense nucleic acids into complexes may be used, for example, polyethylenimine polymers. They may be used in combination with PEG, or conjugated to cyclodextrin and with disulfide linkage to mitigate toxicity.

In other embodiments, biodegradable polymers may be used for the nanoparticles, such as poly(β-amino esters), or poly(amidoamine)s (also optionally including PEG or disulfide linkage in the dendrimer core).

In other embodiments, poly(aspartamide) conjugated to ionizable aminoethylene side chains may be used as delivery vehicle.

The immunogenic or vaccine compositions may be formulated as pharmaceutical compositions. The immunogenic or vaccine compositions can be administered, for example, systemically or locally. Routes for systemic administration in general include, for example, transdermal, oral, parenteral routes, including subcutaneous, intravenous, intramuscular, intraarterial, intradermal and intraperitoneal injections and/or intranasal administration routes. Routes for local administration in general include, for example, topical administration routes but also intradermal, transdermal, subcutaneous, or intramuscular injections or intralesional, intracranial, intrapulmonal, intracardial, intratumoral and sublingual injections. More preferably, vaccines may be administered by an intradermal, subcutaneous, or intramuscular route, preferably by injection, which may be needle-free and/or needle injection.

They may be prepared as a single unit dose, or as a plurality of single unit dose. A single unit dose is a discrete amount of the composition comprising a predetermined amount (for example an immunogenic dose) of the active ingredient.

In addition to the active ingredient, e.g. the RNA molecule, or lipid nanoparticles comprising the RNA molecule, the immunogenic or vaccine compositions may further comprise one or more pharmaceutically acceptable carriers, fillers and diluents.

Pharmaceutically acceptable carriers, fillers and diluents must, of course, have sufficiently high purity and sufficiently low toxicity to make them suitable for administration to a person to be treated. Some examples of compounds which can be used as pharmaceutically acceptable carriers, fillers or constituents thereof are sugars, such as, for example, lactose, glucose, trehalose and sucrose; starches, such as, for example, corn starch or potato starch; dextrose; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; alginic acid.

The choice of a pharmaceutically acceptable carrier is determined, in principle, by the manner in which the immunogenic composition or vaccine is administered. Compositions/vaccines are preferably formulated in liquid or solid form. For example, the immunogenic or vaccine compositions suitable for parenteral administration may comprise the active ingredient combined with a pharmaceutically active carrier, such as sterile water, or sterile isotonic saline. It may also be prepared as injectable formulations, packaged in unit dosage form, such as in ampules or in multi-dose containers. It may also be provided in dry form (powder or granular) for reconstitution with a suitable vehicle prior to parenteral administration.

Methods of Use of the RNA Molecule and their Immunogenic Compositions

The RNA molecule and their immunogenic compositions as described in the previous sections are useful as a vaccine in the treatment and/or prophylaxis of infectious disease or cancer, in particular for the prevention or protection from COVID-19, or flu disorders, typically for protection from severe COVID-19 or severe symptoms of flu, in a human subject in need thereof.

Accordingly, the present disclosure provides compositions (e.g., immunogenic compositions as described in the previous section), methods, kits and reagents for the treatment and/or prophylaxis of infectious diseases or cancer in humans and other mammals.

Methods of Use for Protection from Infectious Diseases

The immunogenic compositions disclosed herein can be used as prophylactic agents against infectious diseases, in particular viral, bacterial or parasitic infectious diseases.

Examples of infectious viral disease include without limitation Flu, Covid-19, papilloma, zika, viral immunodeficiency and AIDS, Ebola, Hepatitis B, Mononucleosis infections.

Examples of infectious bacterial disease include without limitation meningitis, enterocolitis, chronic obstructive pulmonary disease (COPD), streptococcal infections, Pseudomonas infections.

Examples of parasitic disease include without limitation Malaria, Leprosis, Cryptosporidiosis or Leishmania (e.g. leishmaniosis).

In specific embodiments, the immunogenic compositions may be used in medicine as a vaccine to prevent and/or protect from coronavirus disease, in particular COVID-19, more specifically to protect from severe COVID-19.

In specific embodiments, the RNA molecules or immunogenic compositions may be used in medicine as a vaccine to prevent and/or protect from influenza disease, more specifically to protect from severe influenza disease.

In exemplary aspects, the RNA molecules or immunogenic compositions of the present disclosure are used to provide prophylactic protection from infectious diseases, and in particular SARS-COV2 or influenza disease, for example COVID-19 or flu disorder, typically for protection from severe COVID-19 or severe influenza. Prophylactic protection from infectious disease can be achieved following administration of an immunogenic amount of a composition or vaccine of the present disclosure, typically with one or more immunogenic doses. The immunogenic composition can be administered once, twice, three times, four times or more.

As used herein, the term “immunogenic dose” refers to an amount specific to induce an antigen specific immune response in a subject when administering such amount in a subject in need thereof, one or several times.

In some embodiments, the RNA molecules or immunogenic compositions of the present disclosure can be used in a method of preventing a viral infection in a subject, the method comprising administering to said subject at least one immunogenic dose of a RNA molecule or composition as provided herein, typically a composition comprising a RNA molecule encoding a fusion protein comprising the antigenic determinant against the infectious agent.

In some embodiments, the RNA molecules or immunogenic compositions of the present disclosure can be used as a method of inhibiting a primary viral infection in a subject, the method comprising administering to said subject at least one immunogenic dose of a RNA molecule or composition as provided herein, typically a composition comprising a RNA molecule encoding a fusion protein comprising the antigenic determinant against the infectious agent.

In some embodiments, the RNA molecules or immunogenic compositions of the present disclosure can be used as a method of reducing an incidence of an infectious disease, typically coronavirus or influenza severe disease in a subject, the method comprising administering to said subject at least an immunogenic dose of a RNA molecule or composition as provided herein, typically a composition comprising a RNA molecule encoding a fusion protein comprising an antigenic determinant against the infectious agent. In some embodiments, the RNA molecules or immunogenic compositions of the present disclosure can be used as a method of protecting from infectious disease, typically coronavirus severe disease or influenza disease, in a subject, the method comprising administering to said subject at least an immunogenic dose of a RNA molecule or composition as provided herein, typically a RNA molecule encoding a fusion protein comprising an antigenic determinant against the infectious disease.

As used herein, the term “severe COVID-19” refers to either
Moderate Illness: Individuals who show evidence of lower respiratory disease during clinical assessment or imaging and who have an oxygen saturation (SpO2)≥94% on room air at sea level.
Severe Illness: Individuals who have SpO2<94% on room air at sea level, a ratio of arterial partial pressure of oxygen to fraction of inspired oxygen (PaO2/FiO2)<300 mm Hg, a respiratory rate >30 breaths/min, or lung infiltrates >50%.
Critical Illness: Individuals who have respiratory failure, septic shock, and/or multiple organ dysfunction.

In some embodiments, the term “severe COVID-19” refers to COVID-19 with respiratory deficiency symptoms requiring hospitalization.

As used herein, the term “severe influenza” refers to a sudden onset of fever and cough or sore throat) and presenting at least one of the following clinical presentations:
Dyspnea, tachypnea, or hypoxia
Radiological signs of lower respiratory tract disease
Central nervous system involvement (e.g., encephalopathy, encephalitis)
Severe dehydration
Acute renal failure
Septic shock
Exacerbation of underlying chronic disease, including asthma, chronic obstructive pulmonary disease (COPD), chronic hepatic or renal insufficiency, diabetes mellitus, or other cardiovascular conditions,
Any other influenza-related condition or clinical presentation requiring hospital admission.

In some embodiments, the RNA molecules or immunogenic compositions of the present disclosure can be used as a method of protecting from one or more of severe symptoms of an infectious disease, in a subject, the method comprising administering to said subject at least an immunogenic dose of a RNA molecule or composition as provided herein, typically a composition comprising a fusion protein encoding the antigenic determinant against the infectious agent. In some embodiments, the RNA molecules or immunogenic compositions of the present disclosure can be used as a method of inhibiting spread of a virus from a first subject infected with a virus to a second subject not infected with said virus, the method comprising administering to said first subject and said second subject at least one immunogenic dose of a RNA molecule or composition as provided herein, typically a composition comprising encoding a fusion protein comprising an antigenic determinant of said virus.

Some embodiments of the present disclosure provide methods of inducing an antigen specific immune response in a subject, comprising administering to the subject any of the immunogenic compositions as provided herein, in an amount effective to produce an antigen-specific immune response. In some embodiments, an antigen specific immune response comprises total T cell response (in particular CD4+ or CD8+ antigen-specific T cell response) and/or a B cell response (specific anti-antigen immunoglobulin response).

In some embodiments, the immunogenic composition is administered to a subject by intradermal injection, intramuscular injection, or by intranasal administration. In preferred embodiments, the immunogenic composition is administered to a subject by intramuscular injection.

In some embodiments, an effective amount of the RNA molecule is one or more doses of 1 μg to 500 μg, preferably 1 μg to 200 μg.

In specific embodiments, the effective amount of the RNA molecule is one or more doses of 1 to 100 μg, preferably 1 to 50 μg, and 1 to 10 μg.

Methods of Use for Treatment or Protection from Cancer Diseases

In specific embodiments wherein the antigenic determinant of the fusion protein comprises tumor antigen, or tumor mutation derived neoepitopes, the immunogenic compositions disclosed herein can be used as prophylactic or therapeutic agents against cancer disease, in particular for treating solid tumor cancer, or cancer with tumor cells expressing such tumor antigen or neoepitope, as disclosed herein.

Such immunogenic compositions for use in treating cancer may also be referred as cancer vaccines.

The terms “cancer” or “neoplasms” include malignancies of the various organ systems, such as affecting lung, breast, thyroid, blood (lymphoid and myeloid tissues.) gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

In specific embodiments, where the RNA molecule encodes a tumor antigen, the administration of the immunogenic compositions of the present disclosure is capable of inducing an anti-tumor response against tumor expressing said tumor antigen.

Examples of cancer disease include without limitation breast cancer (including triple-negative breast cancer), melanoma, lymphoma, non-small cell lung cancer (NSCLC), prostate, colorectal cancer, bladder cancer, pancreatic cancer, chronic myeloid leukemia, head and neck squamous cell carcinoma, myeloid leukemia, lung squamous cell carcinoma, renal cell carcinoma.

In specific embodiments, the RNA molecules or immunogenic compositions of the present disclosure are used to provide prophylactic protection or anti-tumor efficacy from cancer diseases. Prophylactic protection or anti-tumor response from cancer disease can be achieved following administration of an immunogenic amount of a composition of the present disclosure, typically with one or more therapeutically effective doses. The immunogenic composition can be administered once, twice, three times, four times or more.

In some embodiments, the RNA molecules or immunogenic compositions of the present disclosure can be used in a method of preventing or treating a cancer disease in a subject, the method comprising administering to said subject at least a therapeutically efficient amount of a RNA molecule or composition as provided herein, typically a composition comprising a RNA molecule encoding a fusion protein comprising an antigenic determinant against a tumor expressing said antigenic determinant (tumor antigen).

Said RNA molecules can express a fusion protein with either full-length tumor antigen or fragments thereof or a plurality of epitopes of one or more tumor antigens as the first polypeptide domain of the fusion protein. The expression in vivo of said fusion protein comprising one or more antigenic determinants of one or more tumor antigens allow antigen-presenting cells to simultaneously present or cross-present multiple epitopes with class I and II patient-specific human leukocyte antigen (HLA).

In some embodiments, the RNA molecules or immunogenic compositions of the present disclosure can be used as a method of delaying, stopping or inhibiting the growth of a tumor, or reducing the size or even eliminating a tumor in a subject, the method comprising administering to said subject at least one dose of a therapeutically efficient amount of a RNA molecule or composition as provided herein, typically a composition comprising a RNA molecule encoding a fusion protein comprising the antigenic determinant of a tumor antigen expressed in said tumor.

As used herein in the context of cancer treatment, the term “therapeutically efficient amount” refers to an amount specific to induce an antigen specific anti-tumor response in a subject when administering such amount in a subject in need thereof, one or several times.

Some embodiments of the present disclosure provide methods of inducing an antigen specific anti-tumor response in a subject, comprising administering to the subject any of the immunogenic compositions as provided herein, in an amount effective to produce an antigen-specific anti-tumor response. In some embodiments, an antigen specific anti-tumor response comprises total T cell response (in particular CD4+ or CD8+ antigen-specific T cell response) and/or a B cell response (specific anti-antigen immunoglobulin response).

Some embodiments of the present disclosure provide methods of inducing or increasing a CD8 T cell response against a specific tumor antigen or tumor mutation-derived neoepitope in a subject, said method comprising administering to the subject any of the immunogenic compositions as provided herein, in particular comprising a RNA encoding a fusion protein as disclosed herein wherein the first polypeptide domain comprises a CD8 epitope of said tumor antigen or a tumor mutation-derived neoepitope, in an amount effective to induce or increase a CD8 T cell response against said specific tumor antigen or neoepitope.

In some embodiments, the immunogenic composition for use in treating cancer is administered to a subject by intratumoral, intradermal injection, intramuscular injection, intravenous or by intranasal administration. In preferred embodiments, the immunogenic composition is administered to a subject by intramuscular injection.

In some embodiments, an effective amount of the RNA molecule is one or more doses of 1 μg to 500 μg, preferably 1 μg to 200 μg. In specific embodiments, the effective amount of the RNA molecule is one or more doses of 1 to 100 μg, preferably 1 to 50 μg, and 1 to 10 μg.

In some embodiments, the immunogenic composition for use as a cancer vaccine provides therapeutic or prophylactic treatment against a cancer selected from the group consisting of breast cancer (including triple-negative breast cancer), melanoma, lymphoma, non-small cell lung cancer (NSCLC), prostate, colorectal cancer, bladder cancer, pancreatic cancer, head and neck squamous cell carcinoma, myeloid leukemia, lung squamous cell carcinoma, and renal cell carcinoma.

The immunogenic compositions of the present disclosure for use in treating or preventing from cancer disease, may be administered as a sole active ingredient, or in combination therapy, either simultaneously (i.e. in the same pharmaceutical composition), concurrently (i.e. in separate pharmaceutical composition administered one right after the other in any order) or sequentially in any order. Sequential administration is particularly useful when the therapeutic agents of the combination therapy are in different dosage forms and/or are administered on different dosing schedules, e.g., a chemotherapeutic that is administered at least daily while the immunogenic compositions or cancer vaccines is administered less frequently.

In specific embodiments, the immunogenic composition or cancer vaccine of the present disclosure is administered in combination with another cancer vaccine or immunogenic composition, including peptide-based or DNA-based cancer vaccine.

In specific embodiments, the immunogenic composition or cancer vaccine of the present disclosure is administered in combination with another immunotherapy, in particular comprising immune checkpoint inhibitors (in particular anti-PD1, anti-PDL1 and anti-CTLA4 antibody) as active ingredients.

Examples of such anti-PD1 or anti-PDL1 antibody includes without limitation, nivolumab, pembrolizumab, avelumab, durvalumab, cemiplimab, or atezolizumab.

Examples of such anti-CTLA4 antibody includes without limitation, ipilimumab.

In other specific embodiments, the immunogenic composition or cancer vaccine of the present disclosure is administered in combination chemotherapy.

Chemotherapy includes chemotherapeutic agents selected from alkylating agents, antimetabolites, antimicrotubule agents, topoisomerase inhibitors and cytotoxic antibiotics. Alkylating agents include classical alkylating agents, including nitrogen mustards, nitrosoureas, tetrazines, aziridines, cisplatins and derivatives, and non-classical alkylating agents. Nitrogen mustards include mechlorethamine, cyclophosphamide, melphalan, chlorambucil, ifosfamide and busulfan. Nitrosoureas include N-Nitroso-N-methylurea (MNU), carmustine (BCNU), lomustine (CCNU) and semustine (MeCCNU), fotemustine and streptozotocin. Tetrazines include dacarbazine, mitozolomide and temozolomide. Aziridines include thiotepa, mytomycin and diaziquone (AZQ). Cisplatin and derivatives include cisplatin, carboplatin and oxaliplatin. Non-classical alkylating agents include procarbazine and hexamethylmelamine.

Anti-metabolites include the anti-folates, fluoropyrimidines, deoxynucleoside analogues, and thiopurines. The anti-folates include methotrexate and pemetrexed. The fluoropyrimidines include fluorouracil and capecitabine. The deoxynucleoside analogues include cytarabine, gemcitabine, decitabine, azacitidine, fludarabine, nelarabine, cladribine, clofarabine, and pentostatin. The thiopurines include thioguanine and mercaptopurine.

Anti-microtubule agents include vinca alkaloids, and taxanes. The original vinca alkaloids are natural products that include vincristine and vinblastine. Semi-synthetic vinca alkaloids include vinorelbine, vindesine, and vinflunine. Taxanes include paclitaxel, docetaxel.

Topoisomerase inhibitors include irinotecan and topotecan (derived from camptothecin), or etoposide, doxorubicin, mitoxantrone and teniposide, or novobiocin, merbarone and aclarubicin.

Cytotoxic antibiotics, including anthracyclines and bleomycins, mitomycin C and actinomycin. Anthracyclines include doxorubicin and daunorubicin, epirubicin and idarubicin, pirarubicin, aclarubicin, and mitoxantrone.

In other specific embodiments, the immunogenic composition or cancer vaccine of the present disclosure is administered in combination targeted therapy, such as antibody-drug-conjugates, or targeted immunotherapy such as antibody-based cancer therapy, including for example CD20, HER2 or VEGF targeting antibodies.

One advantage of RNA-based cancer vaccine is the development of personalized treatment, wherein the sequence of the tumor antigen, or fragment or epitope is derived from the sequence of a neoepitope of a tumor of a subject in need of such treatment.

Accordingly, the present disclosure relates to a method a treating a tumor in a subject in need thereof, said method comprising
(i) identifying a specific neoepitope in the tumor of said subject,
(ii) in vitro transcribing or synthesizing an RNA molecule encoding a fusion protein as described herein, wherein said first polypeptide domain includes an amino acid sequence of at least said specific neoepitope,
(iii) producing an immunogenic composition comprising the in vitro transcribed or synthesized RNA molecule,
(iv) administering a therapeutically efficient amount of said immunogenic composition in a subject in need thereof.

The disclosure will be further illustrated by the following embodiments, examples and figures. However, these examples and figures should not be interpreted in any way as limiting the scope of the present disclosure.

### Specific Embodiments

E1. An immunogenic composition for use in the treatment and/or prophylaxis of an infectious disease or cancer, in a subject in need thereof, wherein said immunogenic composition comprises a ribonucleic acid (RNA) molecule comprising an open-reading frame encoding a fusion protein, said fusion protein comprising
(i) a first polypeptide domain comprising either
   a. an antigen or a fragment thereof comprising at least one epitope of said antigen,
   b. a peptide moiety comprising a single epitope of an antigen, or
   c. a plurality of peptide moieties, wherein each peptide moiety comprises an epitope of an antigen and wherein said peptide moieties are fused together, optionally via peptide linker,
and wherein said first polypeptide domain is fused to
(ii) a second polypeptide domain comprising a C4bp-derived oligomerization domain and a positively charged tail.

E2. The immunogenic composition for use of Embodiment E1, wherein the second polypeptide domain is fused C-terminally to said first polypeptide domain, optionally via a peptide linker.

E3. The immunogenic composition for use of E1 or E2, wherein said first polypeptide domain comprises a peptide moiety including at least a CD8 or CD4 T cell epitope of said antigen, preferably a CD8 T cell epitope, optionally, with its corresponding C-terminal and N-terminal natural flanking regions, wherein each optional flanking region consists of 2 to 8 contiguous amino acid residues, typically 5 amino acid residues.

E4. The immunogenic composition for use of Embodiment any of E1-E3, wherein said first polypeptide domain comprises a plurality of peptide moieties which are directly fused together, or indirectly via peptide linkers, and each peptide moiety comprises a fragment of an antigen including (i) an epitope of said antigen and (ii) the corresponding C-terminal and N-terminal natural flanking regions to said epitope, wherein each flanking region consists of 2 to 8 contiguous amino acid residues, typically 5 amino acid residues.

E5. The immunogenic composition for use of any one of Embodiments E1-E4, wherein said first polypeptide domain comprises at least two peptide moieties, each peptide moiety comprising an epitope of an antigen, for example a first peptide moiety comprising a CD8 T cell epitope of an antigen and a second peptide moiety comprising a CD4 T cell epitope of the same antigen.

E6. The immunogenic composition for use of any one of Embodiments E1-E5, wherein said RNA molecule encodes a fusion protein which consists essentially of
(i) a first polypeptide domain essentially consisting of a plurality of peptide moieties being fused together, optionally via peptide linker, each peptide moiety comprising an epitope of an antigen, wherein each peptide moiety essentially consists of a fragment of an antigen of 10 to 30 contiguous amino acid residues,
said first polypeptide domain being fused directly or indirectly via a peptide linker, to,
(ii) a second polypeptide domain essentially consisting of a C4bp oligomerization domain and a positively charged tail.

E7. The immunogenic composition for use of any one of Embodiments E1-E6, wherein said fusion protein forms a heptameric protein after self-assembling.

E8. The immunogenic composition for use of any one of Embodiments E1-E7, wherein said antigen or fragments of antigen or said peptide moiety is selected from viral antigens or their fragments including at least one epitope, for example derived from influenza, or coronavirus, and more preferably selected from the group consisting of influenza nucleoprotein NP or hemagglutinin HA2, coronavirus nucleocapsid N, coronavirus protein spike S, coronavirus RED, and HPV antigen E6 and E7.

E9. The immunogenic composition for use of Embodiment E8, wherein said nucleoprotein NP antigen comprises
(i) a polypeptide of SEQ ID NO:8, or
(ii) an antigenic polypeptide variant having at least 90% identity to SEQ ID NO:8.

E10. The immunogenic composition for use of Embodiment E8 or E9, wherein said fusion protein comprises
(i) an epitope of SEQ ID NO:9,
(ii) an epitope of SEQ ID NO:10,
(iii) a combination of (i) and (ii),
(iv) an antigenic polypeptide variant having at least 90% identity to SEQ ID NO:9 or SEQ ID NO:10 or their combinations.

E11. The immunogenic composition for use of any one of Embodiments E1 to E7, wherein said fusion protein comprises one or more tumor mutation-derived neoepitope(s) or epitope(s) from tumor associated antigen(s), for example as expressed in a tumor of a subject in need of said immunogenic composition.

E12. The immunogenic composition for use of Embodiment E11, wherein said fusion protein comprises one or more tumor epitope(s) of one or more tumor associated antigen(s), preferably selected from the group consisting of MAGE-C1, MAGE-C2, NY-SEO-1, surviving, 5 T4, MUC1, PSA, PSCA, PSMA, STEAP1, PAP, MAGE-C3, tyrosinase, gp100, CT7, MAGE-A1, MAGE-A3, CEA, or antigens in virus-associated tumors, such HPV associated tumors, including E7 HPV antigen, for example as expressed in a tumor of a subject in need of said immunogenic composition.

E13. The immunogenic composition for use of Embodiment E11, wherein said fusion protein comprises one or more tumor mutation-derived neoepitopes selected from the group consisting of neoepitopes from beta catenin (CTNNB1), CDK4, alpha-actinin-4 (ACTN4), nuclear transcription factor γ subunit gamma, Acyl-CoA:lysophosphatidylglycerol acyltransferase, KRAS, Protrudin (ZFYVE27), calcium-dependent secretion activator 2 (CASPS2), B-RAF, and dek-can fusion protein.

E14. The immunogenic composition for use of Embodiment E13, wherein said one or more neoepitopes are selected from the following sequences: SYLDSGIHF (SEQ ID NO: 21) from beta catenin (CTNNB1); ACDPHSGHFV (SEQ ID NO: 22) from CDK4; FIASNGVKLV (SEQ ID NO: 23) from alpha-actinin-4 (ACTN4); AQQITKTEV (SEQ ID NO: 24) from nuclear transcription factor γ subunit gamma, AEPINIQTW (SEQ ID NO: 25) from acyl-CoA:lysophosphatidylglycerol acyltransferase, GADGVGKSAL (SEQ ID NO: 26) from KRAS, EHEGSGPEL (SEQ ID NO: 27) from Protrudin (ZFYVE27), TYDTVHRHL (SEQ ID NO: 28) from calcium-dependent secretion activator 2 (CASPS2), EDLTVKIGDFGLATEKSRWSGSHQFEQLS (SEQ ID NO: 29) from B-RAF, and TMKQICKKEIRRLHQY (SEQ ID NO: 30) from dek-can fusion protein, optionally with their C-terminal and N-terminal flanking regions.

E15. The immunogenic composition for use of any one of Embodiments E1-E14, wherein said C4bp-derived oligomerization domain comprises SEQ ID NO:2, or a functional variant thereof having at least 90% identity to SEQ ID NO:2.

E16. The immunogenic composition for use of any one of Embodiments E1-E15, wherein said positively charged tail comprises the sequence ZXBBBBZ (SEQ ID NO:3), wherein (i) Z is absent or is any amino acid, (ii) X is any amino acid, and (iii) B is an arginine or a lysine, preferably said positively charged tail comprises the sequence of SEQ ID NO:4.

E17. The immunogenic composition for use of any one of Embodiments E1-E16, wherein said second polypeptide domain essentially consists of SEQ ID NO:5, or said second polypeptide domain is a functional variant of SEQ ID NO:5 having at least 90% identity to SEQ ID NO:5.

E18. The immunogenic composition for use of any one of Embodiments E1-E17, wherein said RNA is a messenger RNA, preferably synthetic mRNA molecule, comprising (i) a 5′ cap, (ii) a 5′ untranslated region (UTR), (iii) an open-reading frame that encode the fusion protein and (iv) a poly A tail.

E19. The immunogenic composition for use of any one of Embodiments E1-E18, wherein said RNA molecule is a nucleoside-modified RNA, for example a RNA including pseudouridine or N1-methylpseudouridine.

E20. The immunogenic composition for use of any one of Embodiments E1-E19, further comprising one or more of a pharmaceutically acceptable excipients, a further antigen, or a further nucleic acid encoding an antigen.

E21 The immunogenic composition for use of any one of Embodiments E1-E20, which is formulated in lipid nanoparticle (LNP).

E22. The immunogenic composition for use of any one of Embodiments E1-E21, wherein said immunogenic composition is administered via intramuscular route.

E23. A method for treating or preventing an infectious disease or cancer disorder in a subject in need thereof, said method comprising: administering to said subject an effective amount of an immunogenic composition as defined in any of Embodiments E1-E22.

E24. An immunogenic or vaccine composition comprising a ribonucleic acid (RNA) molecule comprising an open-reading frame encoding a fusion protein, said fusion protein comprising
(i) a first polypeptide domain comprising either
   a. an antigen or a fragment thereof comprising at least one epitope of said antigen, or
   b. a peptide moiety comprising a single epitope of an antigen, or
   c. a plurality of peptide moieties, wherein each peptide moiety comprises an epitope of an antigen and wherein said peptide moieties are fused together, optionally via peptide linker,
and wherein said first polypeptide domain is fused to
(ii) a second polypeptide domain comprising a C4bp-derived oligomerization domain and a positively charged tail.

E25. The immunogenic composition of Embodiment E24, wherein the second polypeptide domain is fused C-terminally to said first polypeptide domain, optionally via a peptide linker.

E26. The immunogenic composition of Embodiment E24 or E25, wherein said first polypeptide domain comprises a peptide moiety including at least a CD8 T cell epitope or a CD4 T cell epitope of said antigen, preferably a CD8 T cell epitope, optionally, with its corresponding C-terminal and N-terminal natural flanking regions, wherein each optional flanking region consists of 3 to 8 contiguous amino acid residues, typically 5 amino acid residues.

E27. The immunogenic composition of any of Embodiments E24-E26, wherein said first polypeptide domain comprises a plurality of peptide moieties which are directly fused together, or indirectly via peptide linkers, and each peptide moiety comprises a fragment of an antigen including (i) an epitope of said antigen and (ii) the corresponding C-terminal and N-terminal natural flanking regions to said epitope, wherein each flanking region consists of 3 to 8 contiguous amino acid residues, typically 5 amino acid residues.

E28. The immunogenic composition of any one of Embodiments E24-E27, wherein said first polypeptide domain comprises at least two peptide moieties, each peptide moiety comprising an epitope of an antigen, for example a first peptide moiety comprising a CD8 T cell epitope of an antigen and a second peptide moiety comprising a CD4 T cell epitope of the same antigen.

E29. The immunogenic composition of any one of Embodiments E24-E28, wherein said RNA molecule encodes a fusion protein which consists essentially of
(iii) a first polypeptide domain essentially consisting of a plurality of peptide moieties being fused together, optionally via peptide linker, each peptide moiety comprising an epitope of an antigen, wherein each peptide moiety essentially consists of a fragment of an antigen of 10 to 30 contiguous amino acid residues,
said first polypeptide domain being fused directly or indirectly via a peptide linker, to,
(iv) a second polypeptide domain essentially consisting of a C4bp oligomerization domain and a positively charged tail.

E30. The immunogenic composition of any one of Embodiments E24-E29, wherein said fusion protein forms a heptameric protein after self-assembling.

E31. The immunogenic composition of any one of Embodiments E24-E30, wherein said antigen or fragments of antigen or said peptide moiety is selected from viral antigens or their fragments including at least one epitope, for example derived from influenza, or coronavirus, and more preferably selected from the group consisting of influenza nucleoprotein NP or hemagglutinin HA2, coronavirus nucleocapsid N, coronavirus protein spike S, coronavirus RED, and HPV antigen E6 and E7.

E32. The immunogenic composition of Embodiment E31, wherein said nucleoprotein NP antigen comprises
(i) a polypeptide of SEQ ID NO:8, or
(ii) an antigenic polypeptide variant having at least 90% identity to SEQ ID NO:8.

E33. The immunogenic composition of Embodiment E31 or E32, wherein said fusion protein comprises
(i) an epitope of SEQ ID NO:9,
(ii) an epitope of SEQ ID NO:10,
(iii) a combination of (i) and (ii),
(iv) an antigenic polypeptide variant having at least 90% identity to SEQ ID NO:9 or SEQ ID NO:10 or their combinations.

E34. The immunogenic composition of any one of Embodiments E24 to E31, wherein said fusion protein comprises one or more tumor mutation-derived neoepitope(s) or epitope(s) from tumor associated antigen(s), for example as expressed in a tumor of a subject in need of said immunogenic composition.

E35. The immunogenic composition of Embodiment E34, wherein said fusion protein comprises one or more tumor epitope(s) of one or more tumor associated antigen(s), preferably selected from the group consisting of MAGE-C1, MAGE-C2, NY-SEO-1, surviving, 5 T4, MUC1, PSA, PSCA, PSMA, STEAP1, PAP, MAGE-C3, tyrosinase, gp100, CT7, MAGE-A1, MAGE-A3, CEA, or antigens in virus-associated tumors, such as HPV associated tumors, including E7 HPV antigen, for example as expressed in a tumor of a subject in need of said immunogenic composition.

E36. The immunogenic composition of Embodiment E34, wherein said fusion protein comprises one or more tumor mutation-derived neoepitopes selected from the group consisting of neoepitopes from beta catenin (CTNNB1), CDK4, alpha-actinin-4 (ACTN4), nuclear transcription factor γ subunit gamma, Acyl-CoA:lysophosphatidylglycerol acyltransferase, KRAS, Protrudin (ZFYVE27), calcium-dependent secretion activator 2 (CASPS2), B-RAF, and dek-can fusion protein.

E37. The immunogenic composition of Embodiment E36, wherein said one or more neoepitopes are selected from the following sequences: SYLDSGIHF (SEQ ID NO: 21) from beta catenin (CTNNB1); ACDPHSGHFV (SEQ ID NO: 22) from CDK4; FIASNGVKLV (SEQ ID NO: 23) from alpha-actinin-4 (ACTN4); AQQITKTEV (SEQ ID NO: 24) from nuclear transcription factor γ subunit gamma, AEPINIQTW (SEQ ID NO: 25) from acyl-CoA:lysophosphatidylglycerol acyltransferase, GADGVGKSAL (SEQ ID NO: 26) from KRAS, EHEGSGPEL (SEQ ID NO: 27) from Protrudin (ZFYVE27), TYDTVHRHL (SEQ ID NO: 28) from calcium-dependent secretion activator 2 (CASPS2), EDLTVKIGDFGLATEKSRWSGSHQFEQLS (SEQ ID NO: 29) from B-RAF, and TMKQICKKEIRRLHQY (SEQ ID NO: 30) from dek-can fusion protein, optionally with their C-terminal and N-terminal flanking regions.

E38. The immunogenic composition of any one of Embodiments E24-E37, wherein said C4bp-derived oligomerization domain comprises SEQ ID NO:2, or a functional variant thereof having at least 90% identity to SEQ ID NO:2.

E39. The immunogenic composition of any one of Embodiments E24-E38, wherein said positively charged tail comprises the sequence ZXBBBBZ (SEQ ID NO:3), wherein (i) Z is absent or is any amino acid, (ii) X is any amino acid, and (iii) B is an arginine or a lysine, preferably said positively charged tail comprises the sequence of SEQ ID NO:4.

E40. The immunogenic composition of any one of Embodiments E24-E39, wherein said second polypeptide domain essentially consists of SEQ ID NO:5, or said second polypeptide domain is a functional variant of SEQ ID NO:5 having at least 90% identity to SEQ ID NO:5.

E41. The immunogenic composition of any one of Embodiments E24-E40, wherein said RNA is a messenger RNA, preferably synthetic mRNA molecule, comprising (i) a 5′ cap, (ii) a 5′ untranslated region (UTR), (iii) an open-reading frame that encode the fusion protein and (iv) a poly A tail.

E42. The immunogenic composition of any one of Embodiments E24-E41, wherein said RNA molecule is a nucleoside-modified RNA, for example a RNA including pseudouridine or N1-methylpseudouridine.

E43. The immunogenic composition of any one of Embodiments E24-E42, further comprising one or more of a pharmaceutically acceptable excipients, a further antigen, or a further nucleic acid encoding an antigen.

E44 The immunogenic composition of any one of Embodiments E24-E43, which is formulated in lipid nanoparticle (LNP).

E45. The immunogenic composition of any one of Embodiments E24-E44, wherein the fusion protein further includes a signal peptide, such as tPA signal peptide.

E46. A method for treating or preventing an infectious disease or cancer disorder in a subject in need thereof, said method comprising: administering to said subject an effective amount of an immunogenic composition as defined in any of Embodiments E24-E45.

E47. A method of inducing or increasing a CD8 or CD4 T cell response against a specific epitope in a subject in need thereof, said method comprising administering an effective amount of an immunogenic composition of any of Embodiments E24-E45.

E48. The method of Embodiment E47, wherein said CD8 or CD4 T cell response is directed against an epitope of a specific tumor associated antigen or tumor mutation derived neoepitope.

### EXAMPLES

### Example 1: Head-to-Head Comparison Between Two mRNA Constructs Containing Influenza Virus Nucleoprotein (NP)-Derived Epitopes Fused or not Fused to OVX313 Heptamerization Domain

The mRNA sequences encoding for one Influenza nucleoprotein (NP) CD8 epitope and one NP CD4 epitope were combined in one construct and tested in mice, with and without OVX313. The CD8 peptide sequence was: ASNENMETM (SEQ ID NO:9), corresponding to positions 366-374 of NP protein sequence. The CD4 peptide sequence was: QVYSLIRPNENPAHK (SEQ ID NO:10), corresponding to positions 311-325 of NP sequence. The constructs were designed in order to contain a combination of the NP CD8 epitope and the CD4 epitope in series, within the same mRNA molecule.

The NP CD8 and NP CD4 epitopes were further designed to include 5 amino acids before and 5 amino acids after the epitope CD8 and CD4 sequences. These 5 amino acids are the flanking regions of the two epitopes as they are naturally found in the native NP full-length sequence. The addition of these flanking regions is aimed at improving the processing of the CD4 and CD8 epitopes by the immune system, once they are translated in the cells after mRNA injection (X Zhao et al, PLoS Pathog. 2020 Sep. 4; 16(9): e1008827.ddoi: 10.1371/journal. ppat.1008827).

The CD8 sequence with the flanking aa (underlined) was: RGVQIASNENMETMESSTL (SEQ ID NO:11). The CD4 sequence with the flanking aa (underlined) was: LLQNSQVYSLIRPNENPAHKSQLVW (SEQ ID NO:12).

Starting from the DNA sequence optimized for expression in mouse, the mRNA constructs were obtained by adding untranslated regions (UTRs) both at 5′ and 3′ of the RNA molecules, a Cap1 chemical capping and a polyA tail. mRNA constructs were then formulated in LNPs.

A positive control was added, consisting of the OVX313-NP protein (OVX836) dosed at 30 μg per mouse. PBS buffer will be used as a negative control. The OVX836 protein (NP protein fused with OVX313) was obtained as described in J Del Campo et al. NPJ Vaccines. 2019 Jan. 23; 4:4. doi: 10.1038/s41541-019-0098-4.

In Table 1, the experimental groups are listed, together with the immunization schedule summary.

TABLE 1
Experiment 1 consisted of 4 groups of 5 mice each. Each group was injected with 50 μL of a
solution containing either 1 μg of mRNA (groups 1 and 2), or with 30 μg of OVX836 (Gr3)
or buffer (Gr4). Mice were injected at Day 1 and Day 28 and sacrificed at Day 35, for sample collection.

| Study name | Group Number - Specimen code - Specimen description | Schedule |
|---|---|---|
| OVX836mRNA-Ia | Gr1 - #7 - CD8-Pep₃₆₆₋₃₇₄ + CD4-Pep₃₁₁₋₃₂₅ mRNA 1 μg | Day 1-Day 28 → |
| | Gr2 - #8 - CD8-Pep₃₆₆₋₃₇₄ + CD4-Pep₃₁₁₋₃₂₅-OVX313 mRNA 1 μg | injections |
| | Gr3 - OVX836 - Protein 30 μg | Day 35 → |
| | Gr4 - Buffer - PBS | sacrifice |

Mice Immunizations

Six-week-old female C57BL/6 mice (Charles River Laboratories, Lyon, France) were used in all experiments. The animals were kept under specific pathogen-free conditions, with ad libitum access to food and water. All animal procedures were approved by the Institutional Animal Care ethics committee of the Plateau de Biologie Expérimental de la Souris (CECCAPP_ENS_2020_019, Lyon, France), and accreditations have been obtained from governmental agencies. Mice were immunized twice, 28 days apart, with 1 μg per construction, under RNAse-free conditions. Immunizations were performed by injection into the gastrocnemius muscle, with both injections being administered in the same hind limb. For immunogenicity studies, seven days after the second immunization, mice were sacrificed to collect the spleens. All samples were processed individually immediately after collection.

Preparation of Murine Spleen Cells

Upon mice sacrifice, spleens were collected from each mouse and processed individually. The spleens were individually transferred into C Tubes and sterile PBS was added before proceeding with the dissociation using gentleMACS™ and following manufacturer's instructions with the appropriate program to dissociate spleens; after tissue dissociation, samples were transferred through a cell strainer and centrifuged. ACK lysis buffer was then added to the pellet for 1 minute. After centrifugation, cells were resuspended in complete α-MEM medium and counted with NucleoCounter® NC-202™ following manufacturer's instructions.

IFNγ-ELISpot Assays

Influenza NP-specific T-cells secreting IFN-γ were enumerated using an IFN-γ ELISpot assay (Mabtech, Sweden). Lymphocytes were isolated from the spleens from individual mice as described above. ELISpot plates were coated with the capture mAb (#3321-2H) then incubated overnight at 4° C. according to the instruction manual of Mabtech. Then 2×105 T-cells were cultured for 20 h at 37° C./5% CO2 with 2 μg/mL of the NP366-374 (GenScript, Netherlands) immunodominant peptide epitope in C57BL/6 mice. Concavalin A (Sigma-Aldrich, France) was used as a positive control and unstimulated splenocytes/lung cells were used as negative controls. Spots were counted with an ELISpot reader system (ASTOR™, MABTECH, Swede). The number of protein- or peptide-reactive cells was represented as spot-forming cells (SFCs) per 2×105 cells per well.

Statistical Analysis

The plotting of data and statistical analysis were performed using GraphPad Prism 9 software. Statistical significance was determined using the non-parametric Kruskal-Wallis test followed by Dunn's multiple comparisons test. Differences were considered significant if the p value was p<0.05.

Results

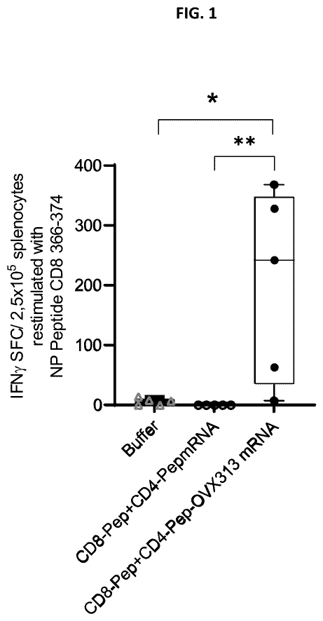

In FIG. 1 the results are summarized. The construct containing the CD8 and CD4 combination only (without the heptamerization domain) showed no activation of IFNγ splenocytes restimulated with the CD8 peptide, being the obtained signal in the Elispot experiment very close to the negative control (buffer). The OVX313-containing constructs surprisingly showed a statistically significant increased response when compared with the other construct. This unexpectedly shows the need of the OVX313 heptamerization domain to trigger a specific CD8 immune response when an mRNA encoding a combination of epitopes is injected in mice.

### Example 2: Head-to-Head Comparison Between Two mRNA Constructs Containing an Influenza Virus Nucleoprotein (NP)-Derived CD8 Epitope Fused or not Fused to OVX313 Heptamerization Domain

The mRNA sequence encoding for one Influenza nucleoprotein (NP) CD8 epitope was tested in mice, with and without OVX313.

The CD8 peptide sequence was: ASNENMETM (SEQ ID NO:9), corresponding to positions 366-374 of NP protein sequence.

The NP CD8 epitope was designed to include 5 amino acids before and 5 amino acids after the CD8 epitope sequence. These 5 amino acids are the flanking regions of the epitope as it is found in the native NP full-length sequence. The addition of these flanking regions is aimed at improving the processing of the CD8 epitope by the immune system, once they are translated in the cells after mRNA injection (X Zhao et al, PLoS Pathog. 2020 Sep. 4; 16(9): e1008827.ddoi: 10.1371/journal.ppat.1008827).

The CD8 sequence with the flanking aa (underlined) was: RGVQIASNENMETMESSTL (SEQ ID NO:11).

Starting from the DNA sequence optimized for expression in mouse, the mRNA constructs were obtained by adding untranslated regions (UTRs) both at 5′ and 3′ of the RNA molecules, a Cap1 chemical capping and a polyA tail. mRNA constructs were formulated in LNPs.

A positive control was added, consisting of the OVX313-NP protein (OVX836) dosed at 30 μg per mouse. PBS buffer will be used as a negative control. The OVX836 protein (NP protein fused with OVX313) was obtained as described in J Del Campo et al. NPJ Vaccines. 2019 Jan. 23; 4:4. doi: 10.1038/s41541-019-0098-4.

In table 2, the experimental groups are listed, together with the immunization schedule details. In this case, all constructs bear tPA (Tissue Plasminogen Activator) leader sequence for secretion of the translated polypeptide.

TABLE 2
Experiment 2 consisted of 4 groups of 5 mice each. Each group was injected
with 50 μL of a solution containing either 1 μg of mRNA (groups
1 and 2), or with 30 μg of OVX836 (Gr3) or buffer (Gr4). Mice were
injected at Day 1 and Day 28 and sacrificed at Day 35, for sample collection.

| Study name | Group Number - Specimen code - Specimen description | Schedule |
|---|---|---|
| OVX836mRNA-Ib | Gr1 - #7 - CD8-Pep₃₆₆₋₃₇₄ mRNA 1 μg | Day 1-Day 28 → |
| | Gr2 - #8 - CD8-Pep₃₆₆₋₃₇₄-OVX313 mRNA 1 μg | injections |
| | Gr3 - OVX836 - Protein 30 μg | Day 35 → |
| | Gr4 - Buffer - PBS | sacrifice |

Mice Immunizations

Six-week-old female C57BL/6 mice (Charles River Laboratories, Lyon, France) were used in all experiments. The animals were kept under specific pathogen-free conditions, with ad libitum access to food and water. All animal procedures were approved by the Institutional Animal Care ethics committee of the Plateau de Biologie Expérimental de la Souris (CECCAPP_ENS_2020_019, Lyon, France), and accreditations have been obtained from governmental agencies. Mice were immunized twice, 28 days apart, with 1 μg per construction, under RNAse-free conditions. Immunizations were performed by injection into the gastrocnemius muscle, with both injections being administered in the same hind limb. For immunogenicity studies, seven days after the second immunization, mice were sacrificed to collect lungs, and splenocytes. All samples were processed individually immediately after collection.

Preparation of Murine Spleen Cells

Upon mice sacrifice, spleens were collected from each mouse and processed individually. The spleens were individually transferred into C Tubes and sterile PBS was added before proceeding with the dissociation using gentleMACS™ and following manufacturer's instructions with the appropriate program to dissociate spleens; after tissue dissociation, samples were transferred through a cell strainer and centrifuged. ACK lysis buffer was then added to the pellet for 1 minute, addition of PBS1X to block the reaction, samples were centrifuged, and pellets were resuspended again in complete α-MEM medium and counted with NucleoCounter® NC-202™ following manufacturer's instructions.

Preparation of Murine Lung Cells

Upon mice sacrifice, lungs were collected from each mouse and minced with sterile surgical blades or scissors. Minced lungs were then individually transferred in a C tube containing 5 mL/lung of digestion medium before proceeding with the tissue dissociation using gentleMACS™ and following manufacturer's instructions with the appropriate program for lung dissociation; after an incubation of 30 mins at 37° C., a further dissociation cycle was applied on gentleMACS™, following manufacturer's instructions. After tissue dissociation, samples were centrifuged and ACK lysis buffer was then added to the pellet for 1 minute. After addition of PBS1X to block the reaction, samples were centrifuged, and pellets were resuspended again in complete α-MEM medium and transferred through a cell strainer. Cells were then counted with NucleoCounter® NC-202™ following manufacturer's instructions.

IFNγ—ELISpot Assays

Influenza NP-specific T-cells secreting IFN-γ were enumerated using an IFN-γ ELISpot assay (Mabtech, Sweden). Lymphocytes were isolated from the spleens and the lungs from individual mice as described above. ELISpot plates were coated with the capture mAb (#3321-2H) then incubated overnight at 4° C. according to the instruction manual of Mabtech. Then 2×10⁵ T-cells were cultured for 20 h at 37° C./5% CO2 with 2 μg/mL of the NP366-374 (GenScript, Netherlands) immunodominant peptide epitope in C57BL/6 mice. Concavalin A (Sigma-Aldrich, France) was used as a positive control and unstimulated splenocytes/lung cells were used as negative controls. Spots were counted with an ELISpot reader system (ASTOR™, MABTECH, Swede). The number of protein- or peptide-reactive cells was represented as spot-forming cells (SFCs) per 2×10⁵ cells per well.

Statistical Analysis

The plotting of data and statistical analysis were performed using GraphPad Prism 9 software. Statistical significance was determined using the non-parametric Kruskal-Wallis test followed by Dunn's multiple comparisons test. Differences were considered significant if the p value was p<0.05.

Results

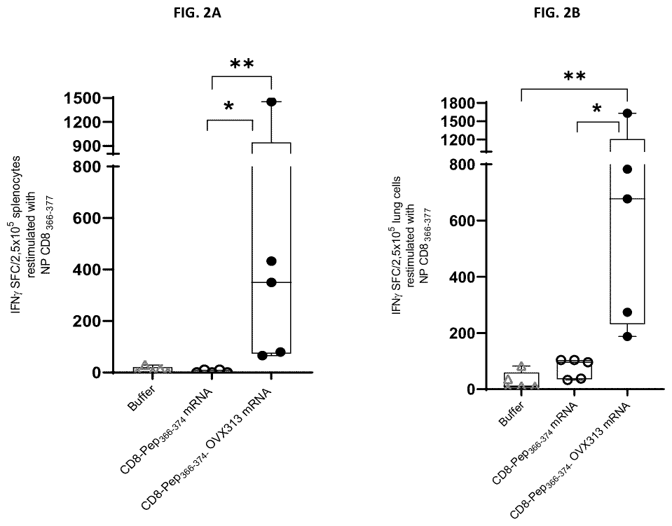

In FIG. 2 the results are summarized. The construct containing the CD8 epitope only (without the heptamerization domain) showed no activation of IFNγ splenocytes restimulated with the CD8 peptide, being the obtained signal in the Elispot experiment very close to the negative control (buffer). The OVX313-containing construct unexpectedly showed a statistically significant increased response when compared with the other construct. This showed the need of the OVX313 heptamerization domain to surprisingly trigger a specific CD8 immune response when an mRNA encoding a combination of epitopes is injected in mice.

### Example 3: Head-to-Head Comparison Between Two mRNA Constructs Containing Influenza Virus Nucleoprotein (NP)-Derived Epitopes Fused to OVX313 Heptamerization Domain with and without tPA Leader Signal Sequence

The mRNA sequences encoding for one Influenza nucleoprotein (NP) CD8 epitope and one NP CD4 epitope were combined in one construct and tested in mice with OVX313, with and without tPA signal sequence. This signal sequence ensures the secretion of the constructs, once translated in the cells after injection. We wanted to see if a secretion signal sequence was able to improve the immune response of this construct: a head-to-head comparison was therefore be done between constructs with and without tPA signal sequence.

The CD8 peptide sequence was: ASNENMETM, corresponding to positions 366-374 of NP protein sequence.

The CD4 peptide sequence was: QVYSLIRPNENPAHK, corresponding to positions 311-325 of NP sequence.

The constructs were designed in order to contain a combination of the NP CD8 epitope and the CD4 epitope in series, within the same mRNA molecule.

The NP CD8 and NP CD4 T cell epitopes were designed to include 5 amino acids before and 5 amino acids after the pure CD8 and CD4 sequences. These 5 amino acids are the flanking regions of the two epitopes as they are in the NP full-length sequence. The addition of these flanking regions is aimed at improving the processing of the CD8 and CD4 epitopes by the immune system, once they are translated in the cells after mRNA injection (X Zhao et al, PLoS Pathog. 2020 Sep. 4; 16(9): e1008827.ddoi: 10.1371/journal.ppat.1008827).

The CD8 sequence with the flanking aa (underlined) was: RGVQIASNENMETMESSTL

The CD4 sequence with the flanking aa (underlined) was:

LLQNSQVYSLIRPNENPAHKSQLVW

Starting from the DNA sequence optimized for expression in mouse, the mRNA constructs were obtained by adding untranslated regions (UTRs) both at 5′ and 3′ of the RNA molecules, a Cap1 chemical capping and a polyA tail. mRNA constructs were formulated in LNPs.

A positive control was added, consisting of the OVX313-NP protein (OVX836) dosed at 30 μg per mouse. PBS buffer will be used as a negative control.

In table 3 below, the experimental groups are listed, together with the immunization schedule summary.

TABLE 3
Experiment 3 consisted of 4 groups of 5 mice each. Each group was injected with 50 μL of a
solution containing either 1 μg of mRNA (groups 1 and 2), or with 30 μg of OVX836 (Gr3)
or buffer (Gr4). Mice were injected at Day 1 and Day 28 and sacrificed at Day 35, for sample collection.

| Study name | Group Number - Specimen code - Specimen description | Schedule |
|---|---|---|
| OVX836mRNA-Ia | Gr1 - #8 - CD8-Pep₃₆₆₋₃₇₄ + CD4-Pep₃₁₁₋₃₂₅-OVX313 mRNA- tPA 1 μg | Day 1-Day 21 → |
| | Gr2 - #9 - CD8-Pep₃₆₆₋₃₇₄ + CD4-Pep₃₁₁₋₃₂₅-OVX313 mRNA 1 μg | injections |
| | Gr3 - Buffer - PBS | Day 28 → |
| | | sacrifice |

Mice Immunizations

Six-week-old female C57BL/6 mice (Charles River Laboratories, Lyon, France) were used in all experiments. The animals were kept under specific pathogen-free conditions, with ad libitum access to food and water. All animal procedures were approved by the Institutional Animal Care ethics committee of the Plateau de Biologie Expérimental de la Souris (CECCAPP_ENS_2020_019, Lyon, France), and accreditations have been obtained from governmental agencies. Mice were immunized twice, 21 days apart, with 1 μg per construction, under RNAse-free conditions. Immunizations were performed by injection into the gastrocnemius muscle, with both injections being administered in the same hind limb. For immunogenicity studies, seven days after the second immunization, mice were sacrificed to collect the spleens. All samples were processed individually immediately after collection.

Preparation of Murine Spleen Cells

Upon mice sacrifice, spleens were collected from each mouse and processed individually. The spleens were individually transferred into C Tubes and sterile PBS was added before proceeding with the dissociation using gentleMACS™ and following manufacturer's instructions with the appropriate program to dissociate spleens; after tissue dissociation, samples were transferred through a cell strainer and centrifuged. ACK lysis buffer was then added to the pellet for 1 minute, addition of PBS1X to block the reaction, samples were centrifuged, and pellets were resuspended again in complete α-MEM medium and counted with NucleoCounter® NC-202™ following manufacturer's instructions.

Preparation of Murine Lung Cells

Upon mice sacrifice, lungs were collected from each mouse and minced with sterile surgical blades or scissors. Minced lungs were then individually transferred in a C tube containing 5 mL/lung of digestion medium before proceeding with the tissue dissociation using gentleMACS™ and following manufacturer's instructions with the appropriate program for lung dissociation; after an incubation of 30 mins at 37° C., a further dissociation cycle was applied on gentleMACS™, following manufacturer's instructions. After tissue dissociation, samples were centrifuged and ACK lysis buffer was then added to the pellet for 1 minute. After addition of PBS1X to block the reaction, samples were centrifuged, and pellets were resuspended again in complete α-MEM medium and transferred through a cell strainer. Cells were then counted with NucleoCounter® NC-202™ following manufacturer's instructions.

IFNγ—ELISpot Assays

Influenza NP-specific T-cells secreting IFN-γ were enumerated using an IFN-γ ELISpot assay (Mabtech, Sweden). Lymphocytes were isolated from the spleens and the lungs from individual mice as described above. ELISpot plates were coated with the capture mAb (#3321-2H) then incubated overnight at 4° C. according to the instruction manual of Mabtech. Then 2×105 T-cells were cultured for 20 h at 37° C./5% CO2 with 2 μg/mL of the NP366-374 (GenScript, Netherlands) immunodominant peptide epitope in C57BL/6 mice. Concavalin A (Sigma-Aldrich, France) was used as a positive control and unstimulated splenocytes/lung cells were used as negative controls. Spots were counted with an ELISpot reader system (ASTOR™, MABTECH, Swede). The number of protein- or peptide-reactive cells was represented as spot-forming cells (SFCs) per 2×105 cells per well

Statistical Analysis

The plotting of data and statistical analysis were performed using GraphPad Prism 9 software. Statistical significance was determined using the non-parametric Kruskal-Wallis test followed by Dunn's multiple comparisons test. Differences were considered significant if the p value was p<0.05.

Results

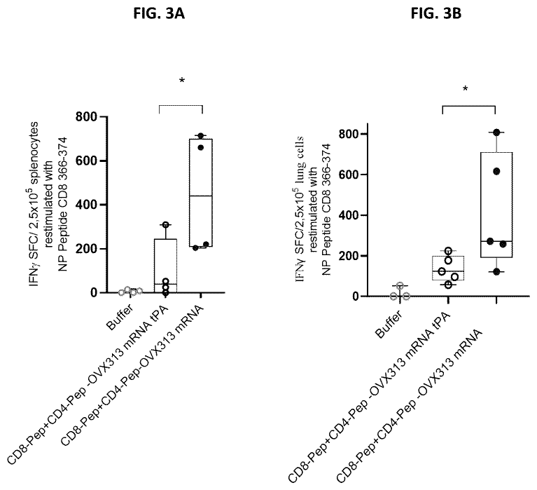

In FIG. 3 the results are summarized. The construct containing the CD8-CD4-OVX313 with the tPA signal sequence showed a better activation of IFNγ splenocytes and lung PBMCs restimulated with the CD8 peptide compared to the negative control. Yet, the construct without tPA showed a statistically significant increased response when compared with the other construct. This shows a better performance of this construct, when the tPA leader sequence is not present in the sequence, when an mRNA encoding a combination of CD8 and CD4 epitopes with OVX313 is injected in mice.

### Example 4: Head-to-Head Comparison Between Two mRNA Constructs Containing an Influenza Virus Nucleoprotein (NP)-Derived Epitope Fused or not Fused to OVX313 Heptamerization Domain

The mRNA sequence encoding for one Influenza nucleoprotein (NP) CD8 epitope was tested in mice, with and without OVX313, both constructs also including a tPA signal sequence. This signal sequence is aimed at ensuring the secretion of the constructs, once translated in the cells after injection.

The CD8 peptide sequence was: ASNENMETM, corresponding to positions 366-374 of NP protein sequence.

The NP CD8 epitopes was designed to include 5 amino acids before and 5 amino acids after the pure CD8 sequence. These 5 amino acids are the same flanking regions of the epitope as they are in the NP full-length sequence. The addition of these flanking regions is aimed at improving the processing of the CD8 epitope by the immune system, once it is translated in the cells after mRNA injection (X Zhao et al, PLoS Pathog. 2020 Sep. 4; 16(9): e1008827.ddoi: 10.1371/journal.ppat.1008827).

The CD8 sequence with the flanking aa (underlined) was: RGVQIASNENMETMESSTL.

Starting from the DNA sequence optimized for expression in mouse, the mRNA constructs were obtained by adding untranslated regions (UTRs) both at 5′ and 3′ of the RNA molecules, a Cap1 chemical capping and a polyA tail. mRNA constructs were formulated in LNPs.

In this experiment, the sequence without OVX313 was built in order to have the same mRNA length as its counterpart with OVX313 (thus excluding any potential contribution to the immune response of the extra mRNA nucleotides of the OVX313-containing construct) but it was coding only for the NP CD8 epitope.

To achieve this, a stop codon was inserted after the NP CD8 epitope encoding sequence followed by a non-sense OVX313 sequence obtained with a one-nucleotide shift in the reading frame, in order to have an untranslated non-sense mRNA sequence after the NP CD8 encoding sequence; the reading frame shift created 4 additional stop codons, that ensured the lack of OVX313 in the final protein product.

PBS buffer was used as a negative control.

In table 4, the experimental groups are listed, together with the immunization schedule summary.

TABLE 4
Experiment 4 consisted of 3 groups of 5 mice each. Each group was injected with
50 μL of a solution containing either 1 μg of mRNA (groups 1 and 2), or buffer (Gr3). Mice were
injected at Day1 and Day 21 and sacrificed at Day 28, for sample collection.

| Study name | Group Number-Specimen Code-Specimen description | Schedule |
|---|---|---|
| OVX836mRNA-II | Gr1-#17-CD8-Pep₃₆₆₋₃₇₄-OVX313 mRNA tPA 1 μg | Day1-Day21 → |
| | Gr2-#19-CD8-Pep₃₆₆₋₃₇₄-STOP codon-non-sense mRNA tPA 1 μg | injections |
| | Gr3-Buffer-PBS | Day28 → |
| | | sacrifice |

Mice Immunizations

Six-week-old female C57BL/6 mice (Charles River Laboratories, Lyon, France) were used in all experiments. The animals were kept under specific pathogen-free conditions, with ad libitum access to food and water. All animal procedures were approved by the Institutional Animal Care ethics committee of the Plateau de Biologie Expérimental de la Souris (CECCAPP_ENS_2020_019, Lyon, France), and accreditations have been obtained from governmental agencies. Mice were immunized twice, 21 days apart, with 1 μg per construct, under RNAse-free conditions. Immunizations were performed by injection into the gastrocnemius muscle, with both injections being administered in the same hind limb. For immunogenicity studies, seven days after the second immunization, mice were sacrificed to collect the spleens and the lungs. All samples were processed individually immediately after collection.

Preparation of Murine Spleen Cells

Upon mice sacrifice, spleens were collected from each mouse and processed individually. The spleens were individually transferred into C Tubes and sterile PBS was added before proceeding with the dissociation using gentleMACS™ and following manufacturer's instructions with the appropriate program to dissociate spleens; after tissue dissociation, samples were transferred through a cell strainer and centrifuged. ACK lysis buffer was then added to the pellet for 1 minute, addition of PBS1X to block the reaction, samples were centrifuged, and pellets were resuspended again in complete α-MEM medium and counted with NucleoCounter® NC-202™ following manufacturer's instructions.

Preparation of Murine Lung Cells

Upon mice sacrifice, lungs were collected from each mouse and minced with sterile surgical blades or scissors. Minced lungs were then individually transferred in a C tube containing 5 mL/lung of digestion medium before proceeding with the tissue dissociation using gentleMACS™ and following manufacturer's instructions with the appropriate program for lung dissociation; after an incubation of 30 mins at 37° C., a further dissociation cycle was applied on gentleMACS™, following manufacturer's instructions. After tissue dissociation, samples were centrifuged and ACK lysis buffer was then added to the pellet for 1 minute. After addition of PBS1X to block the reaction, samples were centrifuged, and pellets were resuspended again in complete α-MEM medium and transferred through a cell strainer. Cells were then counted with NucleoCounter® NC-202™ following manufacturer's instructions.

IFNγ—ELISpot Assays

Influenza NP-specific T-cells secreting IFN-γ were enumerated using an IFN-γ ELISpot assay (Mabtech, Sweden). Lymphocytes were isolated from the spleens and the lungs from individual mice as described above. ELISpot plates were coated with the capture mAb (#3321-2H) then incubated overnight at 4° C. according to the instruction manual of Mabtech. Then 2×105 T-cells were cultured for 20 h at 37° C./5% CO2 with 2 μg/mL of the NP366-374 (GenScript, Netherlands) immunodominant peptide epitope in C57BL/6 mice. Concavalin A (Sigma-Aldrich, France) was used as a positive control and unstimulated splenocytes/lung cells were used as negative controls. Spots were counted with an ELISpot reader system (ASTOR™, MABTECH, Swede). The number of protein- or peptide-reactive cells was represented as spot-forming cells (SFCs) per 2×105 cells per well

Statistical Analysis

The plotting of data and statistical analysis were performed using GraphPad Prism 9 software. Statistical significance was determined using the non-parametric Kruskal-Wallis test followed by Dunn's multiple comparisons test. Differences were considered significant if the p value was p<0.05.

Results

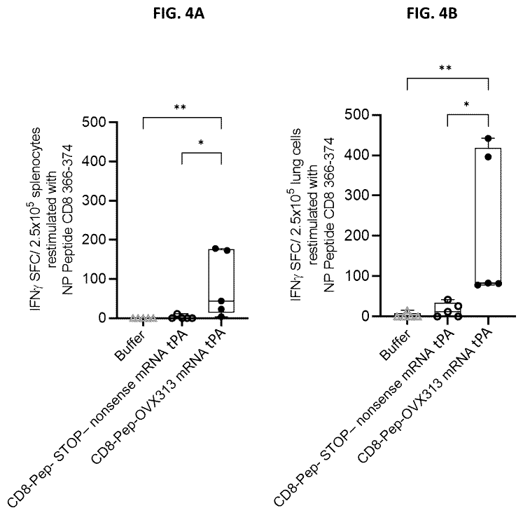

In FIG. 4 the results are summarized. In the Spleen, the construct containing the CD8-OVX313 showed a statistically significant better activation of IFNγ secreting splenocytes compared to its counterpart without OVX313 and compared to the negative control, after restimulation of the splenocytes with the CD8 peptide. Similarly, in the lungs, the CD8-OVX313 construct showed a statistically significant better activation of IFNγ secreting PBMCs compared to its counterpart without OVX313 in PBMCs restimulated with the CD8 peptide. This shows a better performance of the construct with OVX313 where an mRNA encoding an NP CD8 epitope fused to OVX313 is injected in mice, as compared to a similar construct without OVX313 coding sequence.

### Example 5: Head-to-Head Comparison Between Two mRNA Constructs Containing One HPV16 E7 Protein-Derived CD8 Epitope Fused to OVX313 Heptamerization Domain or not

The mRNA sequences encoding for one HPV16 E7-derived CD8 epitope were tested in mice with and without OVX313, with and without tPA signal sequence.

The sequence encoding the CD8 HPV16-E7 epitope was fused at its C-term to OVX313 in a single mRNA molecule and compared head-to-head with the same mRNA sequence without the heptamerization domain OVX313 (replaced by an untranslated nonsense sequence).

The CD8 epitope sequence was designed to include 5 amino acids before and 5 amino acids after the epitope sequence. These 5 amino acids are the flanking regions of the epitope as they are in the HPV16-E7 full-length sequence. The addition of these flanking regions is aimed at improving the processing of the epitope by the immune system, once they are translated in the cells after mRNA injection (X Zhao et al, PLoS Pathog. 2020 Sep. 4; 16(9): e1008827.ddoi: 10.1371/journal.ppat.1008827).

Both constructs were synthesized with and without tPA signal sequence at the N-terminus and compared head-to-head.

The HVP16 E7 CD8 Epitope sequence is SEQ ID NO:31 (RAHYNIVTF) The HPV16 E7 CD8 Epitope sequence including the flanking amino acids (underlined) was the following:

(SEQ ID NO: 33)
QAEPDRAHYNIVTFCCKCD

Starting from the DNA sequence optimized for expression in mouse, the mRNA constructs were obtained by adding untranslated regions (UTRs) both at 5′ and 3′ of the RNA molecules, a Cap1 chemical capping and a polyA tail. mRNA constructs were formulated in LNPs.

In this experiment, 3 μg of each construct were used per mouse.

In table 5, the experimental groups are listed, together with the immunization schedule summary.

TABLE 5
Experiment HPV16-E7-EPITOPE consisted of 5 groups of 5 mice each. Each
mouse was injected with 50 μL of a solution containing 3 μg
of mRNA (groups 1 to 4), or buffer (Gr 5). Mice were injected at Day
1 and Day 21 and sacrificed at Day 28, for sample collection. The
specimen code is the internal construct code for these sequences

| Study name | Group Number - Specimen code - Specimen description | Schedule |
|---|---|---|
| HPV16-E7- | Gr1 - #29 - E7 CD8-OVX313 mRNA | Day 1-Day 21 → |
| EPITOPE | Gr2 - #41 - E7 CD8-OVX313 mRNA - tPA | injections |
| | Gr3 - #30 - E7 CD8-STOP- «nonsense» mRNA | Day 28 → |
| | Gr4 - #32 - E7 CD8- STOP- «nonsense» mRNA - tPA | sacrifice |
| | Gr5 - Buffer - PBS | |

Mice Immunizations

Six-week-old female C57BL/6 mice (Charles River Laboratories, Lyon, France) were used in all experiments. The animals were kept under specific pathogen-free conditions, with ad libitum access to food and water. All animal procedures were approved by the Institutional Animal Care ethics committee of the Plateau de Biologie Expérimental de la Souris (CECCAPP_ENS_2020_019, Lyon, France), and accreditations have been obtained from governmental agencies. Mice were immunized twice, 21 days apart, with 1 μg per construct, under RNAse-free conditions. Immunizations were performed by injection into the gastrocnemius muscle, with both injections being administered in the same hind limb. For immunogenicity studies, seven days after the second immunization, mice were sacrificed to collect the spleens. All samples were processed individually immediately after collection.

Preparation of Murine Spleen Cells

Upon mice sacrifice, spleens were collected from each mouse and processed individually. The spleens were individually transferred into C Tubes and sterile PBS was added before proceeding with the dissociation using gentleMACS™ and following manufacturer's instructions with the appropriate program to dissociate spleens; after tissue dissociation, samples were transferred through a cell strainer and centrifuged. ACK lysis buffer was then added to the pellet for 1 minute, addition of PBS1X to block the reaction, samples were centrifuged, and pellets were resuspended again in complete α-MEM medium and counted with NucleoCounter® NC-202™ following manufacturer's instructions.

IFNγ—ELISpot Assays

HPV16 E7 epitope-specific T-cells secreting IFN-γ were enumerated using an IFN-γ ELISpot assay (Mabtech, Sweden). Lymphocytes were isolated from the spleens from individual mice as described above. ELISpot plates were coated with the capture mAb (#3321-2H) then incubated overnight at 4° C. according to the instruction manual of Mabtech. Then 2×105 T-cells were cultured for 20 h at 37° C./5% C02 with 2 μg/mL of the HPV16 E7 immunodominant peptide in C57BL/6 mice (GenScript, Netherlands). Concavalin A (Sigma-Aldrich, France) was used as a positive control and unstimulated splenocytes/lung cells were used as negative controls. Spots were counted with an ELISpot reader system (ASTOR™, MABTECH, Swede). The number of peptide-reactive cells was represented as spot-forming cells (SFCs) per 2×105 cells per well.

Statistical Analysis

The plotting of data and statistical analysis were performed using GraphPad Prism 9 software. Statistical significance was determined using the non-parametric Kruskal-Wallis test followed by Dunn's multiple comparisons test. Differences were considered significant if the p value was p<0.05.

Results

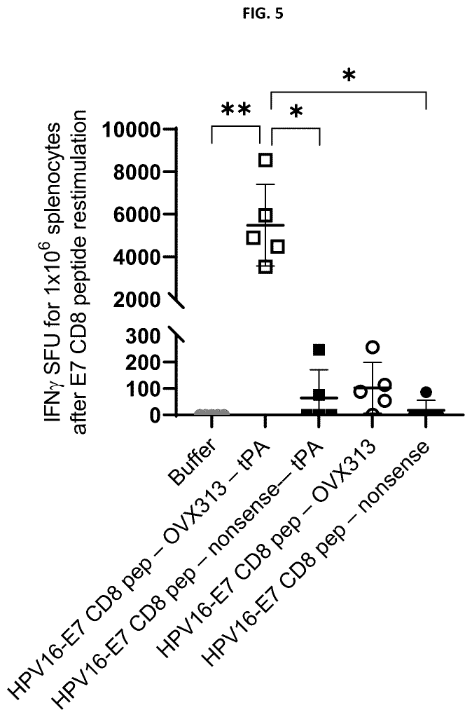

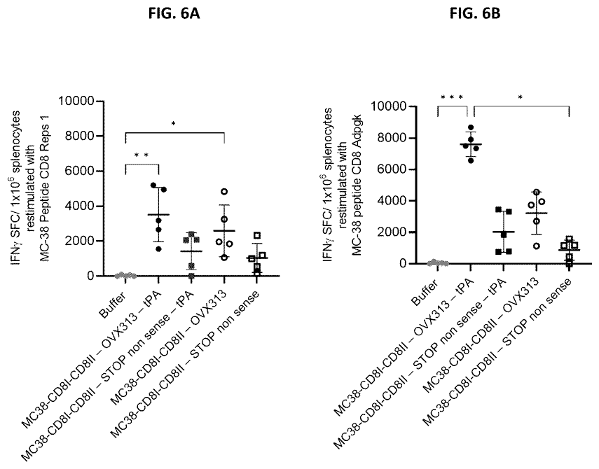

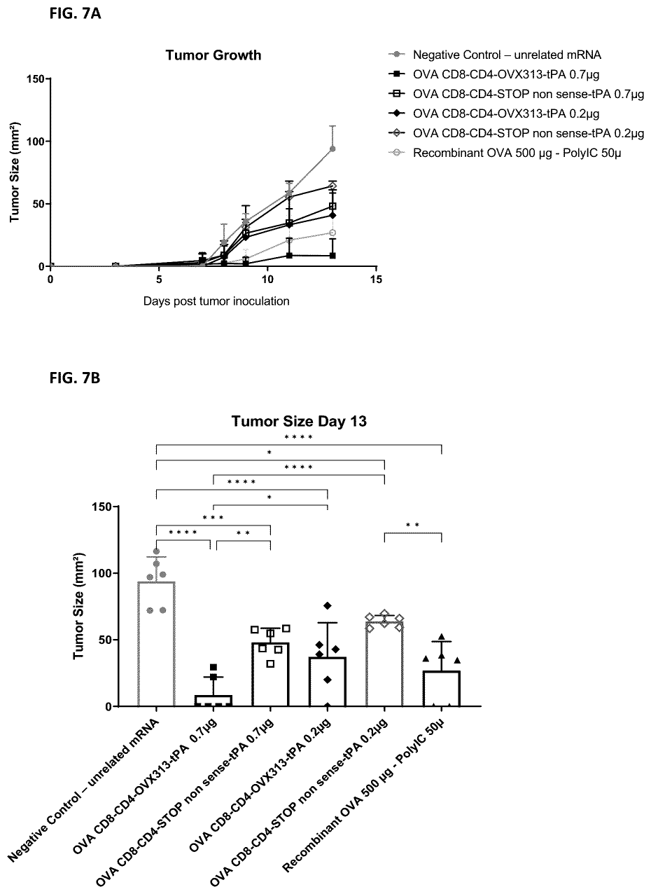

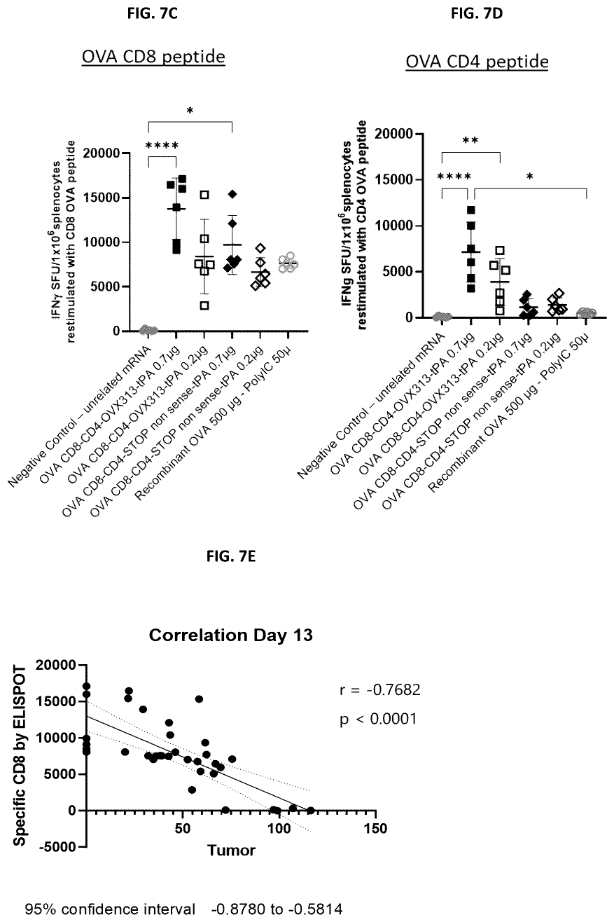

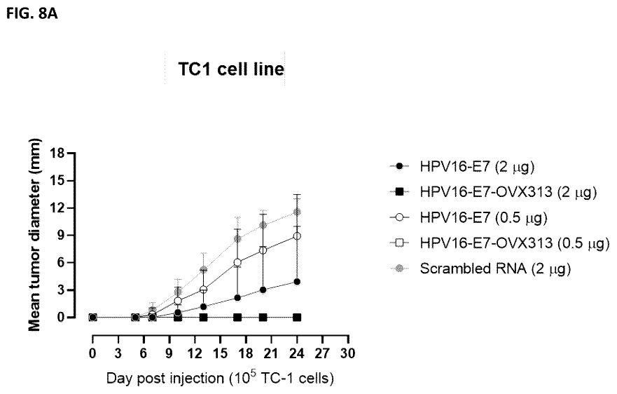

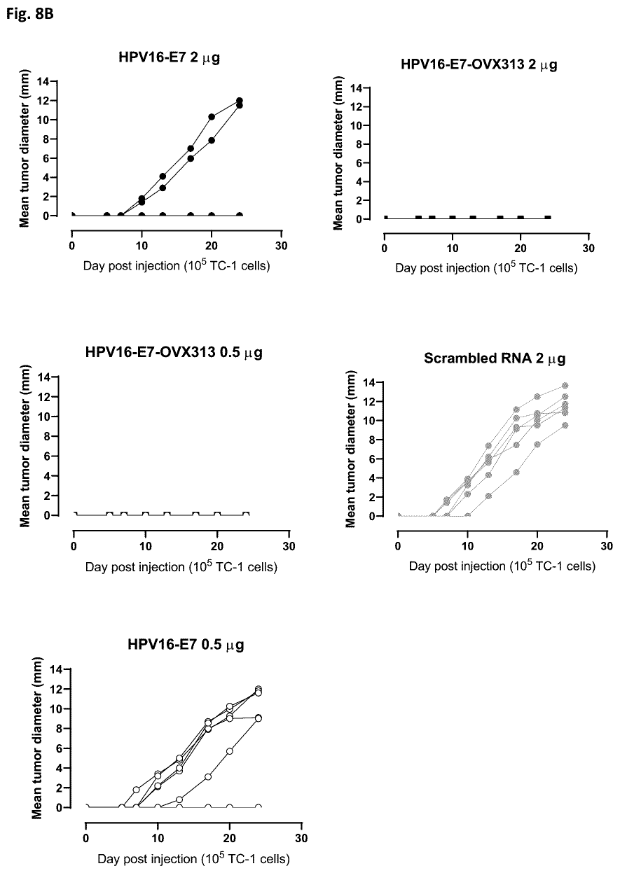

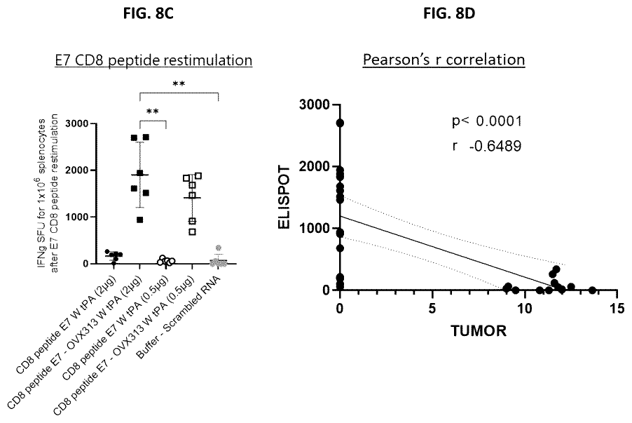

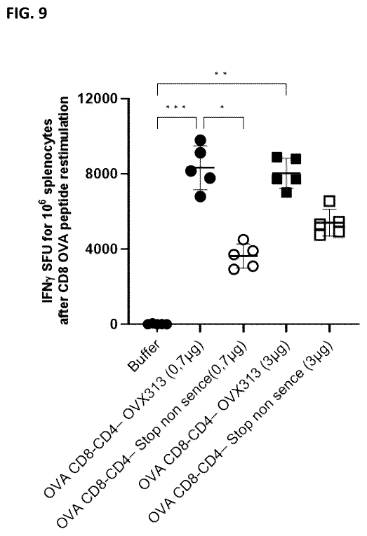

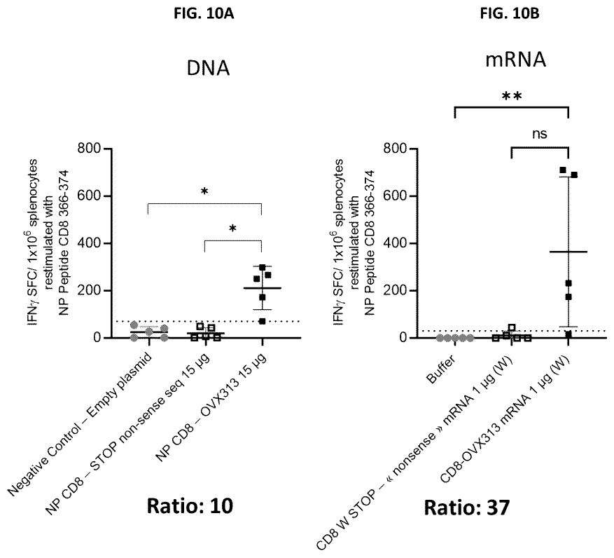

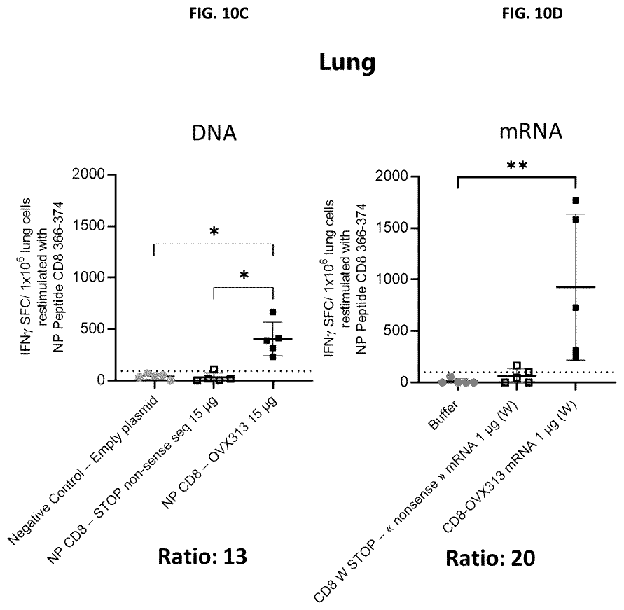

In FIG. 5, the results are summarized. The construct containing the OVX313-E7 CD8 with the tPA signal sequence showed a better activation of IFNγ splenocytes restimulated with the E7 CD8 peptide, compared to the negative control and compared to all the other specimens tested, the level of response being significantly increased compared to the response achieved after vaccination with HPV16E7-CD8-STOP-tPA mRNA (the counterpart without OVX313).

Moreover, this experiment shows a better performance of the construct where the tPA leader sequence is present in the sequence, when an mRNA encoding the HPV16 E7 CD8 epitope fused to OVX313 is injected in mice.

### Example 6: Head-to-Head Comparison Between mRNA Constructs Encoding Two MC38 Murine Tumor CD8 Epitopes Fused or not Fused to OVX313 Heptamerization Domain

The mRNA sequences encoding for two CD8 epitopes from two different mutated antigens of the murine MC38 tumor were tested in mice with and without OVX313, with and without tPA signal sequence. The sequence encoding the CD8 epitopes in series was fused at its C-term to OVX313 in a single mRNA molecule and compared head-to-head with the same mRNA sequence without the heptamerization domain OVX313 (replaced by an untranslated nonsense sequence). Both constructs were synthesized with and without tPA signal sequence at the N-terminus and compared head-to-head. The MC38 CD8 epitopes were designed to include 5 amino acids before and 5 amino acids after the pure CD8 sequences. These 5 amino acids are the flanking regions of the two epitopes as they are in the full-length antigens they are taken from. The addition of these flanking regions is aimed at improving the processing of the epitope by the immune system, once they are translated in the cells after mRNA injection (X Zhao et al, PLoS Pathog. 2020 Sep. 4; 16(9): e1008827.ddoi: 10.1371/journal. ppat.1008827).

The Reps1 CD8 epitope sequence, including the flanking amino acids (underlined) was:

(SEQ ID NO: 34)
ELFRAAQLANDVVLQIMEL.

The Adpgk CD8 epitope sequence, including the flanking amino acids (underlined) was:

(SEQ ID NO: 35)
VHLELASMTNMELMSSIVH.

Starting from the DNA sequence optimized for expression in mouse, the mRNA constructs were obtained by adding untranslated regions (UTRs) both at 5′ and 3′ of the RNA molecules, a Cap1 chemical capping and a polyA tail. mRNA constructs were formulated in LNPs.

In this experiment, 3 μg of each construct were used per mouse.

In table 6, the experimental groups are listed, together with the immunization schedule summary.

TABLE 6
Experiment MC38-2EPITOPES consisted of 5 groups of 5 mice each. Each
group was injected with 50 μL of a solution containing 3 μg
of mRNA (groups 1 to 4), or or buffer (Gr5). Mice were injected at
Day 1 and Day 21 and sacrificed at Day 28, for sample collection.
The specimen code is the internal construct code for these sequences

| Study name | Group Number - Specimen code - Specimen description | Schedule |
|---|---|---|
| MC38- | Gr1 - #23 - MC38-CD8 pep IAQLANDVVL + | Day 1-Day 21 → |
| 2EPITOPES | CD8 pep IIASMTNMELM + STOP - non-sense | injections |
| | Gr2 - #21 - MC38-CD8 pep IAQLANDVVL + | Day 28 → |
| | CD8 pep IIASMTNMELM + OVX313 | sacrifice |
| | Gr3 - #24 - MC38-CD8 pep IAQLANDVVL + | |
| | CD8 pep IIASMTNMELM + STOP - non sense - tPA | |
| | Gr4 - #22 - MC38-CD8 pep IAQLANDVVL + | |
| | CD8 pep IIASMTNMELM + OVX313 - tPA | |
| | Gr5 - Buffer - PBS | |

Mice Immunizations

Six-week-old female C57BL/6 mice (Charles River Laboratories, Lyon, France) were used in all experiments. The animals were kept under specific pathogen-free conditions, with ad libitum access to food and water. All animal procedures were approved by the Institutional Animal Care ethics committee of the Plateau de Biologie Expérimental de la Souris (CECCAPP_ENS_2020_019, Lyon, France), and accreditations have been obtained from governmental agencies. Mice were immunized twice, 21 days apart, with 1 μg per construction, under RNAse-free conditions. Immunizations were performed by injection into the gastrocnemius muscle, with both injections being administered in the same hind limb. For immunogenicity studies, seven days after the second immunization, mice were sacrificed to collect the spleens. All samples were processed individually immediately after collection.

Preparation of Murine Spleen Cells

Upon mice sacrifice, spleens were collected from each mouse and processed individually. The spleens were individually transferred into C Tubes and sterile PBS was added before proceeding with the dissociation using gentleMACS™ and following manufacturer's instructions with the appropriate program to dissociate spleens; after tissue dissociation, samples were transferred through a cell strainer and centrifuged. ACK lysis buffer was then added to the pellet for 1 minute, addition of PBS1X to block the reaction, samples were centrifuged, and pellets were resuspended again in complete α-MEM medium and counted with NucleoCounter® NC-202™ following manufacturer's instructions.

IFNγ—ELISpot Assays

MC38 Reps1 CD8 epitope and MC38 Adpgk CD8 epitope-specific T-cells secreting IFN-γ were enumerated using an IFN-γ ELISpot assay (Mabtech, Sweden). Lymphocytes were isolated from the spleens from individual mice as described above. ELISpot plates were coated with the capture mAb (#3321-2H) then incubated overnight at 4° C. according to the instruction manual of Mabtech. Then 2×105 T-cells were cultured for 20 h at 37° C./5% CO2 with 2 μg/mL of the MC38 Reps1 CD8 epitope or with 2 μg/mL MC38 Adpgk CD8 epitope (GenScript, Netherlands) immunodominant peptides in C57BL/6 mice. Concavalin A (Sigma-Aldrich, France) was used as a positive control and unstimulated splenocytes/lung cells were used as negative controls. Spots were counted with an ELISpot reader system (ASTOR™, MABTECH, Swede). The number of peptide-reactive cells was represented as spot-forming cells (SFCs) per 2×105 cells per well.

Statistical Analysis

The plotting of data and statistical analysis were performed using GraphPad Prism 9 software. Statistical significance was determined using the non-parametric Kruskal-Wallis test followed by Dunn's multiple comparisons test. Differences were considered significant if the p value was p<0.05.

Results

In FIG. 6 the results are summarized. When encoded into an mRNA, OVX313 significantly increases the cellular immune response to a combination of two CD8 epitopes deriving from MC38 murine colon carcinoma cell line. This is true upon the restimulation with both peptides. Moreover, the tPA signal sequence contributed to a statistically significant better immune response of the OVX313-bearing construct, upon restimulation with one of the two CD8 peptides (derived from Adpgk antigen), when compared to its counterpart without tPA. Such improvement is still visible when the restimulation is done with the second peptide, although less evident.

### Example 7: Tumor Growth Rate Reduction Effect on an OVA-Expressing B16 Murine Melanoma Cell Line of an mRNA Construct Encoding One CD8 and One CD4 OVA-Derived Epitope Fused with OVX313, Compared to its Counterpart without OVX313: A Head-to-Head Comparison

The mRNA sequence encoding for two epitopes (one CD4 and one CD8 epitope) from ovalbumin (OVA) was tested in an in vivo experiment to evaluate the tumor growth rate reduction capability against a B16 melanoma cell line expressing OVA. The mRNA constructs contained also the secretion sequence from the human tissue plasminogen activator (tPA) at the N-term. This signal sequence ensures the secretion of the constructs, once translated in the cells after injection. The sequence encoding the CD8-CD4 epitope combination in series was fused at its C-term to OVX313 in a single mRNA molecule and compared head-to-head with the same mRNA sequences without the heptamerization domain OVX313 (replaced by an untranslated nonsense sequence). Both constructs were synthesized with tPA signal sequence at the N-terminus. The CD8 and CD4 epitopes were designed to include 5 amino acids before and 5 amino acids after the peptide sequences. These 5 amino acids are the flanking regions of the two epitopes as they are in the full-length OVA antigen they are taken from. The addition of these flanking regions is aimed at improving the processing of the epitope by the immune system, once they are translated in the cells after mRNA injection (X Zhao et al, PLoS Pathog. 2020 Sep. 4; 16(9): e1008827.ddoi: 10.1371/journal.ppat.1008827).

The OVA CD8 epitope sequence, including the flanking amino acids (underlined) was:

(SEQ ID NO: 36)
LEQLESIINFEKLTEWTS.

The OVA CD4 epitope sequence, including the flanking amino acids (underlined) was:

(SEQ ID NO: 37)
AESLKISQAVHAAHAEINEAGREVVGS.

Starting from the DNA sequence optimized for expression in mouse, the mRNA constructs were obtained by adding untranslated regions (UTRs) both at 5′ and 3′ of the RNA molecules, a Cap1 chemical capping and a polyA tail. mRNA constructs were formulated in LNPs.

The experiment consisted in the subcutaneous injection of 2×10⁵ cells per mouse at d=0, followed by 5 repeated injections of two concentrations of the constructs to be compared. The full-length OVA protein was injected intraperitoneally with polyIC adjuvant as positive control. An uncorrelated mRNA sequence encoding for two flu epitopes fused to OVX313 was used as a negative control.

In this experiment, 0.7 μg and 0.2 μg of each construct were used per mouse, while 500 μg of OVA were mixed with 50 μg of PolyIC in the positive control.

In table 7, the experimental groups are listed, together with the immunization schedule summary.

TABLE 7
Experiment CD4-CD8 OVA EPITOPES consisted of 6 groups of 6 mice each. Each group
was injected with 50 μL of a solution containing 0.7 or 0.2 μg of mRNA (depending
on group), or OVA protein 30 μg formulated with poly IC adjuvant (Gr5). B16-
OVA melanoma cells were subcutaneously implanted at d = 0 and mice were vaccinated
five times from Day 1 every two days and sacrificed at Day 13, for sample collection.
The specimen code is the internal construct code for these sequences

| Study name | Group Number - Specimen code - Specimen description | Schedule |
|---|---|---|
| CD4-CD8 OVA | Gr1 - #6 - CD8 (366-374) + CD4 (311-325) NP peptides + | Day 0 → |
| EPITOPES | OVX313 mRNA with tPA in LNPs - 0.7 g/dose | Tumor Implant |
| | Gr2 - #47 - CD8 (258-265) + CD4 (324-340) OVA peptides + | Days 1, 3, 5, 7, |
| | OVX313 mRNA with tPA in LNPs - 0.7 g/dose | 9 → injections |
| | Gr3 - #48 - CD8 (258-265) + CD4 (324-340) OVA peptidesh + | Day 13 → |
| | STOP - non sense seq mRNA with tPA in LNPs - 0.7 g/dose | sacrifice |
| | Gr4 - #47 - CD8 (258-265) + CD4 (324-340) OVA peptides + | |
| | OVX313 mRNA with tPA in LNPs - 0.2 g/dose | |
| | Gr5 - #48 - CD8 (258-265) + CD4 (324-340) OVA peptidesh + | |
| | STOP - non sense seq mRNA with tPA in LNPs - 0.2 g/dose | |
| | Gr6 - OVA 500 μg - Poly IC 50 μg/dose | |

Mice Treatments

Six-week-old female C57BL/6 mice (Janvier Labs, France) were used in all experiments. The animals were kept under specific pathogen-free conditions, with ad libitum access to food and water. All animal procedures were approved by the French Ministry of Research (agreement number: APAFIS #12905-2018010411002729 v7). Mice were inoculated with 2×105 OVA-B16 cells subcutaneously on the left hip at d=0 and immunized five times starting d=1, 2 days apart, with 0.7 μg or 0.2 μg per construct, under RNAse-free conditions. Immunizations were performed by injection into the right thigh muscle, with all injections being administered in the same hind limb. At d=3, 7, 8, 9, 11 and 13 tumors were measured to obtain growth curves for every group. For immunogenicity studies, at d=13 after tumor implant, mice were sacrificed to collect the spleens. All samples were processed individually immediately after collection.

Preparation of Murine Spleen Cells

Upon mice sacrifice, spleens were collected from each mouse and processed individually. The spleens were individually transferred into C Tubes and sterile PBS was added before proceeding with the dissociation using gentleMACS™ and following manufacturer's instructions with the appropriate program to dissociate spleens; after tissue dissociation, samples were transferred through a cell strainer and centrifuged. ACK lysis buffer was then added to the pellet for 1 minute, addition of PBS1X to block the reaction, samples were centrifuged, and pellets were resuspended again in complete α-MEM medium and counted with NucleoCounter® NC-202™ following manufacturer's instructions.

IFNγ—ELISpot Assays

OVA CD8 epitope and OVA CD4 epitope-specific T-cells secreting IFN-γ were enumerated using an IFN-γ ELISpot assay (Mabtech, Sweden). Lymphocytes were isolated from the spleens from individual mice as described above. ELISpot plates were coated with the capture mAb (#3321-2H) then incubated overnight at 4° C. according to the instruction manual of Mabtech. Then 2×105 T-cells were cultured for 20 h at 37° C./5% CO2 with 2 μg/mL of the OVA CD8 epitope or with 2 μg/mL OVA CD4 epitope (GenScript, Netherlands) immunodominant peptides in C57BL/6 mice. Concavalin A (Sigma-Aldrich, France) was used as a positive control and unstimulated splenocytes/lung cells were used as negative controls. Spots were counted with an ELISpot reader system (ASTOR™, MABTECH, Swede). The number of peptide-reactive cells was represented as spot-forming cells (SFCs) per 2×105 cells per well.

Statistical Analysis

The plotting of data and statistical analysis were performed using GraphPad Prism 9 software. Statistical significance was determined using the non-parametric Kruskal-Wallis test followed by Dunn's multiple comparisons test. Differences were considered significant if the p value was p<0.05.

Results

In FIG. 7 the results are summarized. OVA-CD4-CD8 encoding mRNA constructs display anti-tumor activity at both 0,2 and 0,7 μg/dose (FIG. 7-A).

The addition of OVX313 sequence greatly improves anti-tumor responses, especially when administered at 0,7 μg/dose (FIG. 7-A, B). The vaccine is highly efficient to trigger anti-tumor responses and subsequent tumor growth reduction, with 4 mice out of 6 remaining tumor-free.

The OVA CD4-CD8-OVX encoding mRNA vaccine is more efficient than the positive control PolyIC OVA vaccine that triggers an efficient anti-tumor immunity, albeit with a less robust tumor growth control and a lower tumor free mice number (2/6).

The observations rely on a strong tumor antigen-specific response as the control group, that has received mRNA with an irrelevant Ag (Flu NP epitopes) failed to display any anti-tumor activity.

From an immunological point of view, when encoded into an mRNA, OVX313 significantly increases the cellular immune response to a combination of two CD8 and CD4 epitopes deriving from OVA protein (FIGS. 7-C and D), in agreement with what observed for tumor growth reduction effect.

The Pearson correlation between percent specific CD8 T cells secreting IFNg in the spleen at D13 post tumor inoculation and tumor size was also calculated (FIG. 7-E). A correlation exists between tumor size and specific CD8 cellular response to OVA, where the higher the activation of the immune response, the smaller tumor sizes.

Altogether, these data demonstrate the added value of OVX313 in the design of anti-cancer vaccines.

### Example 8: Tumor Growth Rate Reduction Effect on an HPV16 E6- and HPV16-E7-Expressing TC-1 Murine Lung Tumor Cell Line of an mRNA Construct Encoding One CD8 E7-Derived Epitope Fused with OVX313, Compared to its Counterpart without OVX313: A Head-to-Head Comparison

The mRNA sequence encoding for one CD8 epitope from HPV16 E7 protein was tested in an in vivo experiment to evaluate its tumor growth rate reduction capability against the murine TC-1 lung tumor cell line expressing HPV16-E6 and HPV16-E7 proteins. The sequence encoding the CD8 epitope combination was fused at its C-term to OVX313 in a single mRNA molecule and compared head-to-head with the same mRNA sequence without the heptamerization domain OVX313 (replaced by an untranslated nonsense sequence).

Both constructs were synthesized with the human tissue plasminogen activator (tPA) signal sequence at the N-terminus. The tPA secretion sequence ensures the secretion of the constructs, once translated in the cells after injection. The CD8 epitope was designed to include 5 amino acids before and 5 amino acids after the pure peptide sequences. These 5 amino acids are the flanking regions of the epitope as it is in the full-length E7 antigen it is taken from. The addition of these flanking regions is aimed at improving the processing of the epitope by the immune system, once it is translated in the cells after mRNA injection (X Zhao et al, PLoS Pathog. 2020 Sep. 4; 16(9): e1008827.ddoi: 10.1371/journal.ppat.1008827).

The HPV16-E7 CD8 epitope sequence, including the flanking amino acids (underlined) was:

(SEQ ID NO: 33)
QAEPDRAHYNIVTFCCKCD.

Starting from the DNA sequence optimized for expression in mouse, the mRNA constructs were obtained by adding untranslated regions (UTRs) both at 5′ and 3′ of the RNA molecules, a Cap1 chemical capping and a polyA tail. mRNA constructs were formulated in LNPs.

The experiment consisted in two vaccination 21 days apart with of two concentrations of the constructs to be compared, followed by the subcutaneous injection of 1×105 TC-1 cells per mouse at d=35. An uncorrelated mRNA sequence encoding for one flu epitope fused to OVX313 was used as a negative control.

In this experiment, 2 μg and 0.5 μg of each construct were used per mouse.

In table 8 below, the experimental groups are listed, together with the immunization schedule summary.

TABLE 8
E7-CD8 EPITOPE TC1 consisted of 5 groups of 6 mice each. Each group was
injected twice with 50 μL of a solution containing 2 or 0.5 μg
of mRNA (depending on group). TC1 lung cells were subcutaneously implanted
at d = 35 and mice were sacrificed at Day 59, for sample collection.
The specimen code is the internal construct code for these sequences.

| Study name | Group Number - Specimen code - Specimen description | Schedule |
|---|---|---|
| E7-CD8 | Gr1 - #50 - HPV16-E7 CD8 peptide (RAHYNIVTF) + | Days 0, 21 → |
| EPITOPE | STOP NS mRNA with tPA in LNPs - 2 μg/dose | injections |
| TC1 | Gr2 - #49 - HPV16-E7 CD8 peptide (RAHYNIVTF) + | Day 35 → |
| | OVX313 mRNA with tPA in LNPs - 2 μg/dose | Tumor Implant |
| | Gr3 - #50 - HPV16-E7 CD8 peptide (RAHYNIVTF) + | Day 59 → |
| | STOP NS mRNA with tPA in LNPs - 0.5 μg/dose | sacrifice |
| | Gr4 - #49 - HPV16-E7 CD8 peptide (RAHYNIVTF) + | |
| | OVX313 mRNA with tPA in LNPs- 0.5 μg/dose | |
| | Gr5 - #25 - CD8 NP peptide (366-374) + | |
| | OVX313 mRNA with tPA in LNPs - 2 μg/dose | |

Mice Treatments

Six-week-old female C57BL/6 mice (Janvier Labs, France) were used in all experiments. The animals were kept under specific pathogen-free conditions, with ad libitum access to food and water. All animal procedures were approved by the Italian Ministry of Research (agreement number: CC652.124, Authorization number n° 107/2020). Mice were immunized two times starting d=0, 21 days apart, with 2 μg or 0.5 μg per construct, under RNAse-free conditions. Mice were then grafted with 1×10⁵ TC-1 murine lung tumor cells subcutaneously on the left hip at d=35. Two times a week, tumors were measured to obtain growth curves for every group. For immunogenicity studies, at d=61 after the first immunization, mice were sacrificed to collect the spleens. All samples were processed individually immediately after collection.

Preparation of Murine Spleen Cells

Upon mice sacrifice, spleens were collected from each mouse and processed individually. The spleens were individually transferred into C Tubes and sterile PBS was added before proceeding with the dissociation using gentleMACS™ and following manufacturer's instructions with the appropriate program to dissociate spleens; after tissue dissociation, samples were transferred through a cell strainer and centrifuged. ACK lysis buffer was then added to the pellet for 1 minute, addition of PBS1X to block the reaction, samples were centrifuged, and pellets were resuspended again in complete α-MEM medium and counted with NucleoCounter® NC-202™ following manufacturer's instructions.

IFNγ—ELISpot Assays

HPV16-E7 CD8 epitope-specific T-cells secreting IFN-γ were enumerated using an IFN-γ ELISpot assay (Mabtech, Sweden). Lymphocytes were isolated from the spleens from individual mice as described above. ELISpot plates were coated with the capture mAb (#3321-2H) then incubated overnight at 4° C. according to the instruction manual of Mabtech. Then 2×105 T-cells were cultured for 20 h at 37° C./5% CO2 with 2 μg/mL of the HPV16-E7 CD8 epitope (GenScript, The Netherlands) immunodominant peptides in C57BL/6 mice. Concavalin A (Sigma-Aldrich, France) was used as a positive control and unstimulated splenocytes were used as negative controls. Spots were counted with an ELISpot reader system (ASTOR™, MABTECH, Swede). The number of peptide-reactive cells was represented as spot-forming cells (SFCs) per 2×10⁵ cells per well.

Statistical Analysis

The plotting of data and statistical analysis were performed using GraphPad Prism 9 software. Statistical significance was determined using the non-parametric Kruskal-Wallis test followed by Dunn's multiple comparisons test. Differences were considered significant if the p value was p<0.05.

Results

In FIG. 9 the results are summarized. HPV16-E7 CD8 encoding mRNA construct displays some anti-tumor activity at both 2 and 0.5 μg/dose (FIG. 8-A).

The addition of OVX313 sequence greatly improves anti-tumor responses, at both doses (FIG. 8-A, B).

The vaccine is highly efficient to trigger anti-tumor responses and subsequent tumor growth reduction, with 6 mice out of 6 remaining tumor-free.

The observations rely on a strong tumor antigen-specific response as the control group, that has received mRNA with an irrelevant Ag (Flu NP epitopes) failed to display any anti-tumor activity.

From an immunological point of view, when encoded into an mRNA, OVX313 significantly increases the cellular immune response to the HPV16-E7 CD8 epitope (FIGS. 8-C and D), in agreement with what observed for tumor growth reduction effect.

The Pearson correlation between percent specific CD8 T cells secreting IFNg in the spleen at D61 post first immunization and tumor size was also calculated (FIG. 8-D). A correlation exists between tumor size and specific CD8 cellular response to the HPV16-E7 CD8, where the higher the activation of the immune response, the smaller tumor sizes.

Altogether, these data demonstrate the added value of OVX313 in the design of anti-cancer vaccines.

### Example 9: Head-to-Head Comparison Between mRNA Constructs Encoding a Combination of a CD8 and CD4 OVA Epitopes Fused or not Fused to OVX313 Heptamerization Domain, at Two Different Doses

The mRNA sequences encoding for a combination of a CD8 and a CD4 epitope from Ovalbumin (OVA) were tested in mice with and without OVX313, at two different doses.

The sequence encoding the CD8 and CD4 epitopes in series was fused at its C-term to OVX313 in a single mRNA molecule and compared head-to-head with the same mRNA sequence without the heptamerization domain OVX313 (replaced by an untranslated nonsense sequence). The OVA CD8 and CD4 epitopes were designed to include 5 amino acids before and 5 amino acids after the pure CD8 and CD4 epitope sequences. These 5 amino acids are the flanking regions of the two epitopes as they are in the full-length OVA. The addition of these flanking regions is aimed at improving the processing of the epitope by the immune system, once they are translated in the cells after mRNA injection (X Zhao et al, PLoS Pathog. 2020 Sep. 4; 16(9): e1008827.ddoi: 10.1371/journal.ppat.1008827).

The OVA CD4 epitope sequence, including the flanking amino acids (underlined) was:

(SEQ ID NO: 37)
AESLKISQAVHAAHAEINEAGREVVGS

The OVA CD8 epitope sequence, including the flanking amino acids (underlined) was:

(SEQ ID NO: 36)
LEQLESIINFEKLTEWTS

Starting from the DNA sequence optimized for expression in mouse, the mRNA constructs were obtained by adding untranslated regions (UTRs) both at 5′ and 3′ of the RNA molecules, a Cap1 chemical capping and a polyA tail. mRNA constructs were formulated in LNPs.

In this experiment, 3 μg and 0.7 μg of each construct were used per mouse, to compare the dose-dependent effect.

In table 9 below, the experimental groups are listed, together with the immunization schedule summary.

TABLE 9
Experiment CD8-CD4 OVA EPITOPES Immuno consisted of 5 groups of 5 mice each.
Each group was injected with 50 μL of a solution containing 3 or 0.7 μg of
mRNA (depending on group), or PBS as negative control. Mice were vaccinated
two times, three weeks apart and sacrificed at Day 28, for sample collection.
The specimen code is the internal construct code for these sequences

| Study name | Group Number - Specimen code - Specimen description | Schedule |
|---|---|---|
| CD8-CD4 | Gr1 - #47 - CD8 (258-265) + CD4 (324-340) OVA peptides + | Days 0, 21 → |
| OVA | OVX313 mRNA with tPA in LNPs - 3 μg/dose | injections |
| EPITOPES | Gr2 - #48 - CD8 (258-265) + CD4 (324-340) OVA peptides + | Day 28 → |
| Immuno | STOP -NS seq mRNA with tPA in LNPs - 3 μg/dose | sacrifice |
| | Gr3 - #47 - CD8 (258-265) + CD4 (324-340) OVA peptide + | |
| | OVX313 mRNA with tPA in LNPs - 0.7 μg/dose | |
| | Gr4 - #48 - CD8 (258-265) + CD4 (324-340) OVA peptides + | |
| | STOP - NS seq mRNA with tPA in LNPs - 0.7 μg/dose | |
| | Gr5 - Buffer - PBS | |

Mice Immunizations

Six-week-old female C57BL/6 mice (Charles River Laboratories, Lyon, France) were used in all experiments. The animals were kept under specific pathogen-free conditions, with ad libitum access to food and water. All animal procedures were approved by the Institutional Animal Care ethics committee of the Plateau de Biologie Expérimental de la Souris (CECCAPP_ENS_2022_002, Lyon, France), and accreditations have been obtained from governmental agencies. Mice were immunized twice, 21 days apart, with 1 μg per construction, under RNAse-free conditions. Immunizations were performed by injection into the gastrocnemius muscle, with both injections being administered in the same hind limb. For immunogenicity studies, seven days after the second immunization, mice were sacrificed to collect the spleens. All samples were processed individually immediately after collection.

Preparation of Murine Spleen Cells

Upon mice sacrifice, spleens were collected from each mouse and processed individually. The spleens were individually transferred into C Tubes and sterile PBS was added before proceeding with the dissociation using gentleMACS™ and following manufacturer's instructions with the appropriate program to dissociate spleens; after tissue dissociation, samples were transferred through a cell strainer and centrifuged. ACK lysis buffer was then added to the pellet for 1 minute, addition of PBS1X to block the reaction, samples were centrifuged, and pellets were resuspended again in complete α-MEM medium and counted with NucleoCounter® NC-202™ following manufacturer's instructions.

IFNγ—ELISpot Assays

OVA CD8 and CD4 epitopes-specific T-cells secreting IFN-γ were enumerated using an IFN-γ ELISpot assay (Mabtech, Sweden). Lymphocytes were isolated from the spleens from individual mice as described above. ELISpot plates were coated with the capture mAb (#3321-2H) then incubated overnight at 4° C. according to the instruction manual of Mabtech. Then 2×105 T-cells were cultured for 20 h at 37° C./5% CO2 with 2 μg/mL of the OVA CD4 epitope or with 2 μg/mL OVA CD8 epitope (GenScript, Netherlands) immunodominant peptides in C57BL/6 mice. Concavalin A (Sigma-Aldrich, France) was used as a positive control and unstimulated splenocytes/lung cells were used as negative controls. Spots were counted with an ELISpot reader system (ASTOR™, MABTECH, Swede). The number of peptide-reactive cells was represented as spot-forming cells (SFCs) per 1×106 cells per well.

Statistical Analysis

The plotting of data and statistical analysis were performed using GraphPad Prism 9 software. Statistical significance was determined using the non-parametric Kruskal-Wallis test followed by Dunn's multiple comparisons test. Differences were considered significant if the p value was p<0.05.

Results

In FIG. 9, the number of IFNγ secreting cells observed for splenocytes after restimulation with CD8 OVA peptide is shown. The constructs containing OVX313 triggered a better activation upon CD8 peptide restimulation compared to their counterparts with the STOP-non-sense sequence. A similar difference was observed with the two doses (3 μg and 0.7 μg), although for the OVX313-containing sequence the maximum activation level was achieved already at the lowest dose of 0.7 μg/mouse.

Upon cells restimulation with CD4 peptide, a much lower IFNγ response was detected in the groups immunized with the higher dose compared to the lower dose, for both constructs (with or without OVX313—data not shown). This is a known phenomenon which is described in the literature, where higher antigen doses can suppress the CD4 activation while still showing dose dependent CD8 activation, in an antigen-dependent manner. A good summary of this, with examples, is given in Billeskov R et al. HUMAN VACCINES & IMMUNOTHERAPEUTICS 2019, VOL. 15, NO. 2, 407-411, depending on the antigen. A good CD4 activation was instead observed in the presence of OVA-expressing tumor (see Example 7).

### Example 10. Head-to-Head Comparison Between Two DNA Constructs Containing One Flu NP-Derived CD8 Epitope Fused to OVX313 Heptamerization Domain with tPA Leader Signal Sequence (Comparative Example of a DNA Vaccine, not Part of the Invention)

The DNA sequences encoding for one Flu NP-derived CD8 epitope were tested in mice with OVX313 and with tPA signal sequence. The sequence encoding the Flu NP-derived CD8 epitope was fused at its C-term to OVX313 in a single DNA molecule, cloned in a plasmid and compared head-to-head with the same DNA sequence without the heptamerization domain OVX313 (replaced by an untranslated nonsense sequence). The epitope sequence was designed to include 5 amino acids before and 5 amino acids after the pure CD8 epitope sequence. These 5 amino acids are the flanking regions of the epitope as they are in the Flu NP full-length sequence. The addition of these flanking regions is aimed at improving the processing of the epitope by the immune system, once they are translated in the cells after mRNA injection (X Zhao et al, PLoS Pathog. 2020 Sep. 4; 16(9): e1008827.ddoi: 10.1371/journal. ppat.1008827).

Both constructs were cloned in a pcDNA™3.1 (+) Mammalian Expression Vector plasmid from Thermo Fisher.

The Flu NP CD8 Epitope sequence including the flanking amino acids (underlined) was the following:

(SEQ ID NO: 11)
RGVQIASNENMETMESSTL

In this experiment, 15 μg of each plasmid were used per mouse; the negative control was the empty pcDNA™3.1 (+) Mammalian Expression Vector plasmid.

In table 10, the experimental groups of Experiment ‘NP-EPITOPE-DNA’ are listed, together with the immunization schedule summary.

TABLE 10
Experiment NP-EPITOPE-DNA consisted of 3 groups of 5 mice each.
Each group was injected with 50 μL of a solution containing
50 μg of DNA plasmid. Injections were performed at d =
0 and d = 28 and mice sacrificed at Day 35, for sample collection.

| Study name | Group Number - Specimen description | Schedule |
|---|---|---|
| NP-EPITOPE-DNA | Gr1 - Negative Control-Empty plasmid | Days 8, 28 → |
| | Gr2 - CD8 (366-374) NP peptide + | injections |
| | OVX313 DNA Plasmid - 15 μg/dose | Day 35 → |
| | Gr3 - CD8 (366-374) NP peptide + | sacrifice |
| | STOP - non-sense seq DNA Plasmid - 15 μg/dose | |

Mice Immunizations

Six-week-old female C57BL/6 mice (Charles River Laboratories, Lyon, France) were used in all experiments. The animals were kept under specific pathogen-free conditions, with ad libitum access to food and water. All animal procedures were approved by the Institutional Animal Care ethics committee of the Plateau de Biologie Expérimental de la Souris (CECCAPP_ENS_2020_019, Lyon, France), and accreditations have been obtained from governmental agencies. Mice were immunized twice, 21 days apart, with 15 μg per plasmid. Immunizations were performed by injection into the gastrocnemius muscle, with both injections being administered in the same hind limb. For immunogenicity studies, seven days after the second immunization, mice were sacrificed to collect the spleens and the lungs. All samples were processed individually immediately after collection.

Preparation of Murine Spleen Cells

Upon mice sacrifice, spleens were collected from each mouse and processed individually. The spleens were individually transferred into C Tubes and sterile PBS was added before proceeding with the dissociation using gentleMACS™ and following manufacturer's instructions with the appropriate program to dissociate spleens; after tissue dissociation, samples were transferred through a cell strainer and centrifuged. ACK lysis buffer was then added to the pellet for 1 minute, addition of PBS1X to block the reaction, samples were centrifuged, and pellets were resuspended again in complete α-MEM medium and counted with NucleoCounter® NC-202™ following manufacturer's instructions.

Preparation of Murine Lung Cells

Upon mice sacrifice, lungs were collected from each mouse and minced with sterile surgical blades or scissors. Minced lungs were then individually transferred in a C tube containing 5 mL/lung of digestion medium before proceeding with the tissue dissociation using gentleMACS™ and following manufacturer's instructions with the appropriate program for lung dissociation; after an incubation of 30 mins at 37° C., a further dissociation cycle was applied on gentleMACS™, following manufacturer's instructions. After tissue dissociation, samples were centrifuged, and ACK lysis buffer was then added to the pellet for 1 minute. After addition of complete α-MEM medium to block the reaction, samples were centrifuged, and pellets were resuspended again in complete α-MEM medium and transferred through a cell strainer. Cells were then counted with NucleoCounter® NC-202™ following manufacturer's instructions.

IFNγ—ELISpot Assays

Influenza NP-specific T-cells secreting IFN-γ were enumerated using an IFN-γ ELISpot assay (Mabtech, Sweden). Lymphocytes were isolated from the spleens and the lungs from individual mice as described above. ELISpot plates were coated with the capture mAb (#3321-2H) then incubated overnight at 4° C. according to the instruction manual of Mabtech. Then 2×105 T-cells were cultured for 20 h at 37° C./5% CO2 with 2 μg/mL of the Flu NP366-374 (GenScript, Netherlands) CD8 immunodominant peptide epitope in C57BL/6 mice. Concavalin A (Sigma-Aldrich, France) was used as a positive control and unstimulated splenocytes/lung cells were used as negative controls. Spots were counted with an ELISpot reader system (ASTOR™, MABTECH, Swede). The number of protein- or peptide-reactive cells was represented as spot-forming cells (SFCs) per 1×106 cells per well.

Statistical Analysis

The plotting of data and statistical analysis were performed using GraphPad Prism 9 software. Statistical significance was determined using the non-parametric Kruskal-Wallis test followed by Dunn's multiple comparisons test. Differences were considered significant if the p value was p<0.05.

Results

In FIG. 10, the results are summarized. The DNA construct containing the OVX313-NP CD8 with the tPA signal sequence showed a better activation of IFNγ splenocytes and lung cells restimulated with the NP CD8 peptide compared to the negative control and compared to the level of response being significantly increased compared to the response achieved after vaccination with NP CD8 W-STOP-tPA DNA plasmid (the DNA counterpart without OVX313). The fold increase of the response between the plasmids bearing the sequences with and without OVX313, can be calculated on the average value obtained for the ELISPOT count per each group and is equal to 10 in the spleens and 11 in the lungs.

The same ratio can be calculated for the experiments described in EXAMPLE 2, where the same sequences were compared in an mRNA form. In that case, remarkably, the fold increase was 37 for the spleens and 20 for the lungs, showing a much more powerful effect of OVX313 fusion with the mRNA versions of these constructs as compared to their DNA construct.

### Example 11: OVX033 mRNA

A mRNA sequences encoding for the SARS-Cov2 N antigen fused to OVX313 with and without Tissue plasminogen activator (tPA) signal sequence is synthesized. More specifically, the mRNA comprises a sequence encoding SEQ ID NO:32.

This mRNA sequence may be used for example for treating or preventing from coronavirus infection in a subject in need thereof, typically when such mRNA sequence is formulated in lipid nanoparticle.

### Example 12: OVX836 mRNA

A mRNA sequences encoding for the influenza nucleoprotein (NP) antigen fused to OVX313 with and without Tissue plasminogen activator (tPA) signal sequence is synthesized. More specifically, the mRNA comprises a sequence encoding SEQ ID NO:33.

This mRNA sequence may be used for example for treating or preventing from flu infection in a subject in need thereof, typically when such mRNA sequence is formulated in lipid nanoparticle.

### Example 13: Melanoma Epitope mRNA

RNA sequences encoding for the SYLDSGIHF (SEQ ID NO:21) epitope from beta catenin in melanoma with and without OVX313 is produced. The epitope contains 5-amino-acid flanking regions.

Such RNA sequences may be used to provide a specific T-cell immune response against the epitope, for example for use in decreasing of tumor growth rate of melanoma, typically when such mRNA sequence is formulated in lipid nanoparticle.

### Example 14: Breast Cancer Epitope mRNA

RNA sequences encoding for the TYDTVHRHL (SEQ ID NO:28) from Calcium-dependent secretion activator 2 (Gene Symbol CADPS2) in breast cancer with and without OVX313 is produced. The epitope contains 5-amino-acid flanking regions.

Such RNA sequences may be used to provide a specific T-cell immune response against the epitope, for example for use in decreasing of tumor growth rate of breast cancer, typically when such mRNA sequence is formulated in lipid nanoparticle.

### Example 15: Melanoma Epitope mRNA

RNA sequences encoding for the EDLTVKIGDFGLATEKSRWSGSHQFEQLS (SEQ ID NO:29) from B-RAF in melanoma with and without OVX313 is produced. The epitope contains 5-amino-acid flanking regions.

Such RNA sequences may be used to provide a specific T-cell immune response against the epitope, for example for use in decreasing of tumor growth rate of melanoma, typically when such mRNA sequence is formulated in lipid nanoparticle.

### Example 16: Myeloid Leukemia Epitope mRNA

RNA sequences encoding for the TMKQICKKEIRRLHQY epitope (SEQ ID NO:30) from dek-can fusion protein in myeloid leukemia with and without OVX313 is produced. The epitope contains 5-amino-acid flanking regions.

Such RNA sequences may be used to provide a specific T-cell immune response against the epitope, for example for use in treating myeloid leukemia, typically when such mRNA sequence is formulated in lipid nanoparticle.

### Example 17: MC38 Four-Epitope String mRNA

RNA sequences encoding for epitopes SAIRSYQYV (SEQ ID NO:38) and their 5-amino acid flanking regions from Spire1 gene from the MC38 mouse tumor, MALSTYYAL (SEQ ID NO:39) and their 5-amino acid flanking regions from Nle1 gene from the MC38 mouse tumor, AQLANDVVL (SEQ ID NO:40) and their 5-amino acid flanking regions from Reps1 gene from the MC38 mouse tumor, ASMTNMELM (SEQ ID NO:41) and their 5-amino acid flanking regions from Adpgk gene from the MC38 mouse tumor can be fused together in the order they were listed to form a four-epitope string fused with OVX313.

Such RNA sequences may be used to provide a specific T-cell immune response against each of the four epitopes, for example for use in decreasing of tumor growth rate of MC38 in mice, typically when such mRNA sequences are formulated in lipid nanoparticles.

### Example 18: MC38 Seven-Epitope String mRNA

RNA sequences encoding for epitopes SNFHFMCAL (SEQ ID NO:42) and their 5-amino acid flanking regions from Wbp7 gene from the MC38 mouse tumor, QIYAFLQGF (SEQ ID NO:43) and their 5-amino acid flanking regions from Hace1 gene from the MC38 mouse tumor, FALMNLKAL (SEQ ID NO:44) and their 5-amino acid flanking regions from Tmem135 gene from the MC38 mouse tumor, SAIRSYQYV (SEQ ID NO:38) and their 5-amino acid flanking regions from Spire1 gene from the MC38 mouse tumor, MALSTYYAL (SEQ ID NO:39) and their 5-amino acid flanking regions from Nle1 gene from the MC38 mouse tumor, AQLANDVVL (SEQ ID NO:40) and their 5-amino acid flanking regions from Reps1 gene from the MC38 mouse tumor, ASMTNMELM (SEQ ID NO:41) and their 5-amino acid flanking regions from Adpgk gene from the MC38 mouse tumor can be fused together in the order they were listed to form a seven-epitope string fused with the OVX313 RNA sequence.

Such RNA sequence may be used to provide a specific T-cell immune response against each of the seven epitopes, for example for use in decreasing of tumor growth rate of MC38 in mice, typically when such mRNA sequence is formulated in lipid nanoparticles.

### Example 19: Human Melanoma Fifteen-Epitope String mRNA for Use in Inducing a T-Cell Specific Immune Response Against Melanoma Cells in Human

RNA sequences encoding for epitopes GVYPMPGTQK (SEQ ID NO:45) and their 5-amino acid flanking regions from NCAPH2 gene of human melanoma, SYLDSGIHF (SEQ ID NO:21) and their 5-amino acid flanking regions from CTNNB1 gene of human melanoma, KINKNPKYKK (SEQ ID NO:47) and their 5-amino acid flanking regions from MYO1B gene of human melanoma, ACDPHSGHFV (SEQ ID NO:22) and their 5-amino acid flanking regions from CDK4 gene of human melanoma, VRTLLSQVNK (SEQ ID NO:49) and their 5-amino acid flanking regions from CIT gene of human melanoma, RFLEYLPLRF (SEQ ID NO:50) and their 5-amino acid flanking regions from DCAKD gene of human melanoma, KLKFVTLVF (SEQ ID NO:51) and their 5-amino acid flanking regions from ACPP gene of human melanoma, DMKARQKALV (SEQ ID NO:52) and their 5-amino acid flanking regions from FAM50B gene of human melanoma, LPNEYAFVTT (SEQ ID NO:53) and their 5-amino acid flanking regions from COL22A1 gene of human melanoma, FPKKIQMLA (SEQ ID NO:54) and their 5-amino acid flanking regions from DDX3X gene of human melanoma, LRAAFFGKCF (SEQ ID NO:55) and their 5-amino acid flanking regions from VPS16 gene of human melanoma, YPVIFKSIM (SEQ ID NO:56) and their 5-amino acid flanking regions from TBX4 gene of human melanoma, VTEKLQPTY (SEQ ID NO:57) and their 5-amino acid flanking regions from ITGA9 gene of human melanoma, WRNILLLSLH (SEQ ID NO:58) and their 5-amino acid flanking regions from CASP1 gene of human melanoma, EVLPFFLFF (SEQ ID NO:59) and their 5-amino acid flanking regions from AFMID gene of human melanoma can be fused together to form a fifteen-epitope string fused with the OVX313 RNA sequence.

Such RNA sequences may be used to provide a specific T-cell immune response against each of the 15 epitopes, for example for use in decreasing of tumor growth rate of human melanoma, typically when such mRNA sequences are formulated in lipid nanoparticles.

### Example 20: Human Melanoma Twenty-Epitope String mRNA for Use in Inducing a T-Cell Specific Immune Response Against Melanoma Cells in Human

RNA sequences encoding for epitopes QTNPVTLQY (SEQ ID NO:60) and their 5-amino acid flanking regions from the HELZ2 gene of human melanoma, TLYSLTLLY (SEQ ID NO:61) and their 5-amino acid flanking regions from the CENPL gene of human melanoma, FLIYLDVSV (SEQ ID NO:62) and their 5-amino acid flanking regions from the WDR46 gene of human melanoma, FFYLLDFTF (SEQ ID NO:63) and their 5-amino acid flanking regions from the PRDX3 gene of human melanoma, IMQTLAGELY (SEQ ID NO:64) and their 5-amino acid flanking regions from the GCN1 L1 of human melanoma, GVYPMPGTQK (SEQ ID NO:45) and their 5-amino acid flanking regions from NCAPH2 gene of human melanoma, SYLDSGIHF (SEQ ID NO:21) and their 5-amino acid flanking regions from CTNNB1 gene of human melanoma, KINKNPKYKK (SEQ ID NO:47) and their 5-amino acid flanking regions from MYO1B gene of human melanoma, ACDPHSGHFV (SEQ ID NO:22) and their 5-amino acid flanking regions from CDK4 gene of human melanoma, VRTLLSQVNK (SEQ ID NO:49) and their 5-amino acid flanking regions from CIT gene of human melanoma, RFLEYLPLRF (SEQ ID NO:50) and their 5-amino acid flanking regions from DCAKD gene of human melanoma, KLKFVTLVF (SEQ ID NO:51) and their 5-amino acid flanking regions from ACPP gene of human melanoma, DMKARQKALV (SEQ ID NO:52) and their 5-amino acid flanking regions from FAM50B gene of human melanoma, LPNEYAFVTT (SEQ ID NO:53) and their 5-amino acid flanking regions from COL22A1 gene of human melanoma, FPKKIQMLA (SEQ ID NO:54) and their 5-amino acid flanking regions from DDX3X gene of human melanoma, LRAAFFGKCF (SEQ ID NO:55) and their 5-amino acid flanking regions from VPS16 gene of human melanoma, YPVIFKSIM (SEQ ID NO:56) and their 5-amino acid flanking regions from TBX4 gene of human melanoma, VTEKLQPTY (SEQ ID NO:57) and their 5-amino acid flanking regions from ITGA9 gene of human melanoma, WRNILLLSLH (SEQ ID NO:58) from CASP1 gene of human melanoma, EVLPFFLFF (SEQ ID NO:59) from AFMID gene of human melanoma can be fused together to form a twenty-epitope string fused with the OVX313 RNA sequence. Each epitope contains 5-amino-acid flanking regions derived from the sequence of the antigen they come from.

Such RNA sequence may be used to provide a specific T-cell immune response against each of the twenty epitopes, for example for use in decreasing of tumor growth rate of human melanoma, typically when such mRNA sequence is formulated in lipid nanoparticles.

### Example 21: Human Melanoma Thirty-Five-Epitope String mRNA for Use in Inducing a T-Cell Specific Immune Response Against Melanoma Cells in Human

RNA sequences encoding for epitopes TRSSGSHFVF (SEQ ID NO:65) and their 5-amino acid flanking regions from POLA2 gene of human melanoma, VLLGVKLFGV (SEQ ID NO:66) and their 5-amino acid flanking regions from COL18A1 gene of human melanoma, ALYGFVPVL (SEQ ID NO:67) and their 5-amino acid flanking regions from GANAB gene of human melanoma, ILTGLNYEV (SEQ ID NO:68) and their 5-amino acid flanking regions from NSDHL gene of human melanoma, FMPDFDLHL (SEQ ID NO:69) and their 5-amino acid flanking regions from AHNAK gene of human melanoma TESPFEQHI (SEQ ID NO:70) and their 5-amino acid flanking regions from FAM3C gene of human melanoma, RLFPGLTIKI (SEQ ID NO:71) and their 5-amino acid flanking regions from KIF2C gene of human melanoma, CILGKLFTK (SEQ ID NO:72) and their 5-amino acid flanking regions from CDK12 gene of human melanoma, LTDDRLFTCY (SEQ ID NO:73) and their 5-amino acid flanking regions from PLEKHM2 gene of human melanoma, GEEDGAGGHSL (SEQ ID NO:74) and their 5-amino acid flanking regions from RECQL5 gene of human melanoma, GQFLTPNSH (SEQ ID NO:75) and their 5-amino acid flanking regions from TFDP2 gene of human melanoma, RVSTLRVSL (SEQ ID NO:76) and their 5-amino acid flanking regions from GNB5 gene of human melanoma, WLIRETQPITK (SEQ ID NO:53) and their 5-amino acid flanking regions from XPNPEP1 gene of human melanoma, GLLDEDFYA (SEQ ID NO:46) and their 5-amino acid flanking regions from UGGT2 gene of human melanoma, SLADEAEVYL (SEQ ID NO:48) and their 5-amino acid flanking regions from GAS7 gene of human melanoma, QTNPVTLQY (SEQ ID NO:60) and their 5-amino acid flanking regions from the HELZ2 gene of human melanoma, TLYSLTLLY (SEQ ID NO:61) and their 5-amino acid flanking regions from the CENPL gene of human melanoma, FLIYLDVSV (SEQ ID NO:62) and their 5-amino acid flanking regions from the WDR46 gene of human melanoma, FFYLLDFTF (SEQ ID NO:63) and their 5-amino acid flanking regions from the PRDX3 gene of human melanoma, IMQTLAGELY (SEQ ID NO:64) and their 5-amino acid flanking regions from the GCN1L1 of human melanoma, GVYPMPGTQK (SEQ ID NO:45) and their 5-amino acid flanking regions from NCAPH2 gene of human melanoma, SYLDSGIHF (SEQ ID NO:21) and their 5-amino acid flanking regions from CTNNB1 gene of human melanoma, KINKNPKYKK (SEQ ID NO:47) and their 5-amino acid flanking regions from MYO1B gene of human melanoma, ACDPHSGHFV (SEQ ID NO:22) and their 5-amino acid flanking regions from CDK4 gene of human melanoma, VRTLLSQVNK (SEQ ID NO:49) and their 5-amino acid flanking regions from CIT gene of human melanoma, RFLEYLPLRF (SEQ ID NO:50) and their 5-amino acid flanking regions from DCAKD gene of human melanoma, KLKFVTLVF (SEQ ID NO:51) and their 5-amino acid flanking regions from ACPP gene of human melanoma, DMKARQKALV (SEQ ID NO:52) and their 5-amino acid flanking regions from FAM50B gene of human melanoma, LPNEYAFVTT (SEQ ID NO:53) and their 5-amino acid flanking regions from COL22A1 gene of human melanoma, FPKKIQMLA (SEQ ID NO:54) and their 5-amino acid flanking regions from DDX3X gene of human melanoma, LRAAFFGKCF (SEQ ID NO:55) and their 5-amino acid flanking regions from VPS16 gene of human melanoma, YPVIFKSIM (SEQ ID NO:56) and their 5-amino acid flanking regions from TBX4 gene of human melanoma, VTEKLQPTY (SEQ ID NO:57) and their 5-amino acid flanking regions from ITGA9 gene of human melanoma, WRNILLLSLH (SEQ ID NO:58) and their 5-amino acid flanking regions from CASP1 gene of human melanoma, EVLPFFLFF (SEQ ID NO:59) and their 5-amino acid flanking regions from AFMID gene of human melanoma can be fused together to form a thirty-five-epitope string fused with the OVX313 RNA sequence.

Such RNA sequence may be used to provide a specific T-cell immune response against each of the 35 epitopes, for example for use in decreasing of tumor growth rate of human melanoma, typically when such mRNA sequence is formulated in lipid nanoparticles.

### Example 22: Useful Sequences for Practicing the Invention

TABLE 11
Brief description of the sequences

| SEQ ID | | |
|---|---|---|
| NO: | Type* | Brief Description** |
| 1 | aa | Amino acid sequence of the nucleocapsid N antigen (sequence as |
| | | used in OVX033 without methionine) |
| 2 | aa | Amino acid sequence of the hybrid C4bp oligomerization domain |
| | | without the C-terminal positively charged tail (including Glutamate as |
| | | last residue) |
| 3 | aa | Amino acid sequence of the C-terminal positively charged tail |
| | | (ZXBBBBZ) |
| 4 | aa | Amino acid sequence of the C-terminal positively charged tail |
| | | (GRRRRRS) |
| 5 | aa | OVX313 full amino acid sequence without GS linker |
| 6 | aa | OVX033 full amino acid sequence without methionine |
| 7 | aa | Amino acid sequence of tPA signal sequence |
| 8 | aa | Amino acid sequence of the nucleoprotein NP antigen (sequence as |
| | | used in OVX836 without methionine) |
| 9 | aa | Amino acid sequence of NP366-374 |
| 10 | aa | Amino acid sequence of NP311-325 |
| 11 | aa | The NP366-374 with the flanking regions as used in the examples |
| 12 | aa | The NP311-325 with the flanking regions as used in the examples |
| 13 | nt | Coding sequence mRNA of SEQ ID NO: 8 (NP of OVX836) |
| 14 | nt | Coding sequence mRNA of NP366-374 |
| 15 | nt | Coding sequence mRNA of NP311-325 |
| 16 | nt | The coding sequence mRNA of NP366-374 with the flanking regions |
| | | as used in the examples |
| 17 | nt | The coding sequence mRNA of NP311-325 with the flanking regions |
| | | as used in the examples |
| 18 | aa | The amino acid sequence of the fusion protein with the two peptide |
| | | CD8 and CD4 epitopes of NP fused to OVX313 as used in the |
| | | examples |
| 19 | nt | The coding sequence mRNA of the fusion protein with the two |
| | | peptides CD8 and CD4 epitopes of NP as used in the examples |
| 20 | nt | Coding sequence RNA of OVX033 (full-length) without tPA signal |
| | | sequence |
| 21 | aa | SYLDSGIHF from beta catenin (CTNNB1) (in melanoma) |
| 22 | aa | ACDPHSGHFV from CDK4 (in melanoma) |
| 23 | aa | FIASNGVKLV from alpha-actinin-4 (ACTN4) (in non-small cell lung |
| | | cancer) |
| 24 | aa | AQQITKTEV from nuclear transcription factor Y subunit gamma (in |
| | | non-small cell lung cancer), |
| 25 | aa | AEPINIQTW from acyl-CoA:lysophosphatidylglycerol |
| | | acyltransferase (in bladder cancer) |
| 26 | aa | GADGVGKSAL from KRAS (in Colorectal cancer) |
| 27 | aa | EHEGSGPEL from Protrudin (ZFYVE27) (in pancreatic cancer) |
| 28 | aa | TYDTVHRHL from calcium-dependent secretion activator 2 |
| | | (CASPS2) in breast cancer |
| 29 | aa | EDLTVKIGDFGLATEKSRWSGSHQFEQLS from B-RAF in |
| | | melanoma |
| 30 | aa | TMKQICKKEIRRLHQY from dek-can fusion protein in myeloid |
| | | leukemia |
| 31 | aa | Amino acid sequence of HPV-E7 CD8 epitope |
| 32 | aa | Amino acid sequence of human Ig kappa light chain V-III region |
| | | signal peptide |
| 33 | aa | Amino acid sequence of HPV16 E7 CD8 epitope with the flanking |
| | | regions |
| 34 | aa | Reps1 CD8 epitope sequence, including the flanking amino acids |
| | | (underlined) |
| 35 | aa | Adpgk CD8 epitope sequence, including the flanking amino acids |
| | | (underlined) |
| 36 | aa | The OVA CD8 epitope sequence, including the flanking amino acids |
| | | (underlined) |
| 37 | aa | The OVA CD4 epitope sequence, including the flanking amino acids |
| | | (underlined) |
| 38 | aa | SAIRSYQYV epitope from Spire1 gene of the MC38 mouse tumor |
| 39 | aa | MALSTYYAL epitope from Nle1 gene of the MC38 mouse tumor |
| 40 | aa | AQLANDVVL epitope from Reps1 gene of the MC38 mouse tumor |
| 41 | aa | ASMTNMELM epitope from Adpgk gene of the MC38 mouse tumor |
| 42 | aa | SNFHFMCAL epitope from Wbp7 gene from the MC38 mouse tumor |
| 43 | aa | QIYAFLQGF epitope from Hace1 gene from the MC38 mouse tumor |
| 44 | aa | FALMNLKAL epitope from Tmem135 gene from the MC38 mouse |
| | | tumor |
| 45 | aa | GVYPMPGTQK epitope from NCAPH2 gene of human melanoma |
| 46 | aa | GLLDEDFYA epitope from UGGT2 gene of human melanoma |
| 47 | aa | KINKNPKYKK epitope from MYO1B gene of human melanoma |
| 48 | aa | SLADEAEVYL epitope from GAS7 gene of human melanoma |
| 49 | aa | VRTLLSQVNK epitope from CIT gene of human melanoma |
| 50 | aa | RFLEYLPLRF epitope from DCAKD gene of human melanoma |
| 51 | aa | KLKFVTLVF epitope from ACPP gene of human melanoma |
| 52 | aa | DMKARQKALV epitope from FAM50B gene of human melanoma |
| 53 | aa | WLIRETQPITK epitope from XPNPEP1 gene of human melanoma |
| 54 | aa | FPKKIQMLA epitope from DDX3X gene of human melanoma |
| 55 | aa | LRAAFFGKCF epitope from VPS16 gene of human melanoma |
| 56 | aa | YPVIFKSIM epitope from TBX4 gene of human melanoma |
| 57 | aa | VTEKLQPTY epitope from ITGA9 gene of human melanoma |
| 58 | aa | WRNILLLSLH epitope from CASP1 gene of human melanoma |
| 59 | aa | EVLPFFLFF epitope from AFMID gene of human melanoma |
| 60 | aa | QTNPVTLQY epitope from the HELZ2 gene of human melanoma |
| 61 | aa | TLYSLTLLY epitope from the CENPL gene of human melanoma, |
| 62 | aa | FLIYLDVSV epitope from the WDR46 gene of human melanoma, |
| 63 | aa | FFYLLDFTF epitope from the PRDX3 gene of human melanoma, |
| 64 | aa | IMQTLAGELY epitope from the GCN1L1 of human melanoma |
| 65 | aa | TRSSGSHFVF epitope from POLA2 gene of human melanoma |
| 66 | aa | VLLGVKLFGV epitope from COL18A1 gene of human melanoma |
| 67 | aa | ALYGFVPVL epitope from GANAB gene of human melanoma |
| 68 | aa | ILTGLNYEV epitope from NSDHL gene of human melanoma |
| 69 | aa | FMPDFDLHL epitope from AHNAK gene of human melanoma |
| 70 | aa | TESPFEQHI epitope from FAM3C gene of human melanoma |
| 71 | aa | RLFPGLTIKI epitope from KIF2C gene of human melanoma |
| 72 | aa | CILGKLFTK epitope from CDK12 gene of human melanoma |
| 73 | aa | LTDDRLFTCY epitope from PLEKHM2 gene of human melanoma |
| 74 | aa | GEEDGAGGHSL epitope from RECQL5 gene of human melanoma |
| 75 | aa | GQFLTPNSH epitope from TFDP2 gene of human melanoma |
| 76 | aa | RVSTLRVSL epitope from GNB5 gene of human melanoma |

*″aa″ refers to amino acid and ″nt″ refers to nucleotide
**All sequences are disclosed without start codon or methionine

TABLE 12
Sequence Listing

| | |
|---|---|
| 1 | SDNGPQNQRNAPRITFGGPSDSTGSNQNGERSGARSKQRRPQGLPNNTASWFT |
| | ALTQHGKEDLKFPRGQGVPINTNSSPDDQIGYYRRATRRIRGGDGKMKDLSPRW |
| | YFYYLGTGPEAGLPYGANKDGIIWVATEGALNTPKDHIGTRNPANNAAIVLQLPQG |
| | TTLPKGFYAEGSRGGSQASSRSSSRSRNSSRNSTPGSSRGTSPARMAGNGGDA |
| | ALALLLLDRLNQLESKMSGKGQQQQGQTVTKKSAAEASKKPRQKRTATKAYNVT |
| | QAFGRRGPEQTQGNFGDQELIRQGTDYKHWPQIAQFAPSASAFFGMSRIGMEVT |
| | PSGTWLTYTGAIKLDDKDPNFKDQVILLNKHIDAYKTFPPTEPKKDKKKKADETQA |
| | LPQRQKKQQTVTLLPAADLDDFSKQLQQSMSSADSTQA |
| 2 | KKQGDADVCGEVAYIQSVVSDCHVPTAELRTLLEIRKLFLEIQKLKVE |
| 3 | ZXBBBBZ |
| 4 | GRRRRRS |
| 5 | KKQGDADVCGEVAYIQSVVSDCHVPTAELRTLLEIRKLFLEIQKLKVEGRRRRRS |
| 6 | SDNGPQNQRNAPRITFGGPSDSTGSNQNGERSGARSKQRRPQGLPNNTASWFT |
| | ALTQHGKEDLKFPRGQGVPINTNSSPDDQIGYYRRATRRIRGGDGKMKDLSPRW |
| | YFYYLGTGPEAGLPYGANKDGIIWVATEGALNTPKDHIGTRNPANNAAIVLQLPQG |
| | TTLPKGFYAEGSRGGSQASSRSSSRSRNSSRNSTPGSSRGTSPARMAGNGGDA |
| | ALALLLLDRLNQLESKMSGKGQQQQGQTVTKKSAAEASKKPRQKRTATKAYNVT |
| | QAFGRRGPEQTQGNFGDQELIRQGTDYKHWPQIAQFAPSASAFFGMSRIGMEVT |
| | PSGTWLTYTGAIKLDDKDPNFKDQVILLNKHIDAYKTFPPTEPKKDKKKKADETQA |
| | LPQRQKKQQTVTLLPAADLDDFSKQLQQSMSSADSTQAGSKKQGDADVCGEVA |
| | YIQSVVSDCHVPTAELRTLLEIRKLFLEIQKLKVEGRRRRRS |
| 7 | DAMKRGLCCVLLLCGAVFVSPSQEIHARFRR |
| 8 | ASQGTKRSYEQMETDGERQNATEIRASVGKMIGGIGRFYIQMCTELKLSDYEGRL |
| | IQNSLTIERMVLSAFDERRNKYLEEHPSAGKDPKKTGGPIYRRVNGKWMRELILYD |
| | KEEIRRIWRQANNGDDATAGLTHMMIWHSNLNDATYQRTRALVRTGMDPRMCSL |
| | MQGSTLPRRSGAAGAAVKGVGTMVMELVRMIKRGINDRNFWRGENGRKTRIAY |
| | ERMCNILKGKFQTAAQKAMMDQVRESRNPGNAEFEDLTFLARSALILRGSVAHKS |
| | CLPACVYGPAVASGYDFEREGYSLVGIDPFRLLQNSQVYSLIRPNENPAHKSQLV |
| | WMACHSAAFADLRVLSFIKGTKVVPRGKLSTRGVQIASNENMETMESSTLELRSR |
| | YWAIRTRSGGNTNQQRASAGQISIQPTFSVQANLPFDRTTVMAAFTGNTEGRTSD |
| | MRTEIIRMMESARPEDVSFQGRGVFELSDEKAASPIVPSFDMSNEGSYFFGDNAE |
| | EYDN |
| 9 | ASNENMETM |
| 10 | QVYSLIRPNENPAHK |
| 11 | RGVQIASNENMETMESSTL |
| 12 | LLQNSQVYSLIRPNENPAHKSQLVW |
| 13 | GCGACUAAGGGCACGAAACGCAGCUACGAACAAAUGGAAACCGACGGUGAG |
| | CGUCAAAAUGCAACCGAAAUCCGCGCUAGCGUCGGCAAGAUGAUCGACGGC |
| | AUCGGCCGUUUUUACAUUCAGAUGUGCACCGAGCUGAAGCUGAGCGAUUAC |
| | GAGGGUCGUCUGAUUCAGAAUAGCUUGACGAUCGAGCGUAUGGUGUUGAG |
| | CGCGUUCGAUGAGCGCCGCAACAAAUAUCUGGAAGAACAUCCGAGCGCCGG |
| | UAAAGAUCCGAAGAAAACCGGUGGCCCUAUCUACCGUCGUGUUGAUGGCAA |
| | GUGGCGUCGCGAGCUGAUUCUGUAUGACAAAGAAGAAAUUCGCCGUAUUUG |
| | GCGCCAGGCGAAUAAUGGUGACGACGCGACCGCGGGUUUAACGCACAUGA |
| | UGAUCUGGCAUUCCAACCUGAACGAUGCGACGUAUCAACGUACCCGUGCGC |
| | UGGUGCGUACCGGCAUGGACCCACGUAUGUGCUCGCUGAUGCAAGGUUCC |
| | ACCCUGCCUCGUCGUAGCGGUGCUGCCGGUGCGGCAGUGAAAGGUGUCGG |
| | CACGAUGGUCAUGGAACUUAUCCGCAUGAUUAAGCGCGGUAUCAAUGAUCG |
| | UAAUUUCUGGCGCGGUGAGAAUGGUCGUCGUACCCGUAUUGCGUAUGAGC |
| | GUAUGUGCAACAUUCUGAAGGGUAAAUUCCAGACCGCGGCACAGCGUACGA |
| | UGGUCGACCAAGUUCGCGAGUCUCGUAACCCGGGCAAUGCUGAGUUUGAA |
| | GAUCUGAUUUUCCUGGCGCGUAGCGCCCUGAUUCUGCGUGGCUCGGUUGC |
| | GCACAAAUCUUGUCUGCCGGCCUGCGUCUAUGGUAGCGCGGUGGCAUCCG |
| | GUUACGACUUUGAGCGUGAGGGUUAUAGCUUGGUUGGCAUUGACCCGUUU |
| | CGCCUGCUGCAGAACAGCCAGGUGUACAGCCUGAUCCGUCCAAAUGAGAAC |
| | CCGGCACACAAGUCCCAACUGGUUUGGAUGGCAUGUCAUAGCGCGGCUUU |
| | CGAAGAUCUGCGUGUGUCUAGCUUUAUCCGCGGUACCAAAGUUGUGCCGC |
| | GUGGCAAGCUGAGCACGCGUGGUGUGCAAAUCGCCAGCAACGAAAACAUGG |
| | AAACCAUGGAAUCUUCAACCCUGGAGCUGCGUAGCCGUUACUGGGCGAUUC |
| | GCACCCGCAGCGGUGGCAAUACCAACCAGCAACGUGCGAGCAGCGGCCAGA |
| | UCAGCAUUCAACCGACUUUUAGCGUUCAGCGUAAUCUGCCGUUCGACCGCC |
| | CGACGAUCAUGGCAGCCUUUACCGGUAACACCGAGGGUCGCACUAGCGACA |
| | UGCGCACCGAAAUCAUUCGCCUGAUGGAGAGCGCCCGUCCGGAAGAUGUC |
| | AGCUUCCAGGGUCGUGGUGUUUUCGAGCUGAGCGACGAGAAAGCGACCUC |
| | CCCGAUCGUCCCGAGCUUUGACAUGUCUAACGAGGGCAGCUACUUUUUCG |
| | GUGAUAAUGCAGAAGAGUACGAUAACUAA |
| 14 | GCCAGCAACGAAAACAUGGAAACCAUG |
| 15 | CAGGUGUACAGCCUGAUCCGUCCAAAUGAGAACCCGGCACACAAG |
| 16 | CGUGGUGUGCAAAUCGCCAGCAACGAAAACAUGGAAACCAUGGAAUCUUCA |
| | ACCCUG |
| 17 | CUGCUGCAGAACAGCCAGGUGUACAGCCUGAUCCGUCCAAAUGAGAACCCG |
| | GCACACAAGUCCCAACUGGUUUGG |
| 18 | RGVQIASNENMETMESSTLLLQNSQVYSLIRPNENPAHKSQLVWGSKKQGDADV |
| | CGEVAYIQSVVSDCHVPTAELRTLLEIRKLFLEIQKLKVEGRRRRRS |
| 19 | CGUGGUGUGCAAAUCGCCAGCAACGAAAACAUGGAAACCAUGGAAUCUUCA |
| | ACCCUGCUGCUGCAGAACAGCCAGGUGUACAGCCUGAUCCGUCCAAAUGAG |
| | AACCCGGCACACAAGUCCCAACUGGUUUGGGGCAGCAAGAAACAGGGUGAU |
| | GCUGACGUGUGCGGCGAAGUGGCAUAUAUCCAGAGCGUCGUGAGCGAUUG |
| | UCACGUUCCGACGGCAGAGUUGCGCACGCUGUUGGAAAUCCGUAAGCUGU |
| | UCUUGGAGAUUCAAAAGCUCAAAGUUGAGGGUCGUCGUCGCAGACGUUCCU |
| | AA |
| 20 | AGCGACAAUGGCCCACAGAAUCAACGUAAUGCACCGAGAAUCACCUUUGGC |
| | GGCCCGAGCGAUAGCACGGGUAGCAACCAGAACGGCGAGCGCAGCGGUGC |
| | GCGUUCUAAACAGCGCCGUCCGCAAGGUCUGCCGAAUAACACGGCGUCGU |
| | GGUUUACGGCACUGACCCAGCAUGGUAAGGAAGAUUUGAAGUUUCCGCGU |
| | GGCCAGGGUGUCCCGAUCAAUACCAACAGCUCACCUGAUGACCAGAUCGGC |
| | UAUUAUCGUCGCGCCACGCGCCGUAUCCGCGGUGGCGAUGGCAAGAUGAA |
| | AGACUUAAGCCCGCGUUGGUACUUCUAUUACCUGGGUACGGGUCCAGAGG |
| | CUGGUUUGCCGUAUGGUGCUAACAAAGACGGCAUUAUCUGGGUGGCGACC |
| | GAGGGUGCCCUGAACACCCCGAAAGAUCACAUUGGUACUCGUAACCCUGCG |
| | AACAACGCCGCGAUUGUGCUGCAACUGCCGCAGGGUACCACCCUGCCGAAG |
| | GGUUUCUAUGCAGAGGGCAGCCGUGGUGGCAGCCAGGCGAGCAGCCGUAG |
| | CAGCAGCCGUUCCCGCAAUUCUAGCCGCAAUAGCACGCCGGGUUCCAGCC |
| | GUGGUACGUCCCCGGCACGUAUGGCAGGCAAUGGUGGCGAUGCGGCACUG |
| | GCGCUGCUGUUGCUGGACCGCCUGAACCAACUGGAGAGCAAGAUGAGCGG |
| | CAAGGGUCAGCAGCAGCAAGGUCAGACUGUGACCAAAAAGUCCGCGGCAGA |
| | GGCCAGCAAGAAACCACGUCAGAAACGUACCGCGACCAAAGCGUACAAUGU |
| | UACCCAGGCCUUCGGUCGCCGUGGCCCUGAGCAAACCCAAGGUAAUUUCG |
| | GUGACCAAGAACUGAUCCGCCAGGGCACGGACUACAAGCAUUGGCCGCAGA |
| | UUGCGCAGUUUGCACCGAGCGCUAGCGCGUUCUUCGGUAUGUCCCGCAUU |
| | GGUAUGGAAGUGACCCCGUCUGGCACCUGGCUGACCUACACUGGUGCAAU |
| | CAAAUUGGACGAUAAAGACCCGAACUUUAAAGACCAAGUCAUCCUCCUGAAC |
| | AAGCACAUUGAUGCGUACAAGACCUUCCCGCCGACGGAACCGAAAAAAGAC |
| | AAAAAGAAAAAAGCUGACGAAACCCAAGCAUUGCCGCAACGUCAAAAGAAAC |
| | AGCAAACGGUUACCCUGCUUCCGGCAGCGGAUCUGGACGAUUUUAGCAAGC |
| | AACUGCAACAAUCUAUGUCGAGCGCGGACUCCACCCAGGCCGGUAGCAAGA |
| | AACAGGGCGAUGCCGAUGUCUGCGGCGAAGUUGCGUACAUUCAGAGCGUC |
| | GUGAGCGACUGUCACGUUCCGACGGCUGAGCUGCGUACCCUGCUGGAAAU |
| | UCGUAAGCUGUUCCUGGAAAUCCAAAAGCUGAAAGUUGAGGGUCGCCGUCG |
| | UCGCCGUAGCUAA |
| 21 | SYLDSGIHF |
| 22 | ACDPHSGHFV |
| 23 | FIASNGVKLV |
| 24 | AQQITKTEV |
| 25 | AEPINIQTW |
| 26 | GADGVGKSAL |
| 27 | EHEGSGPEL |
| 28 | TYDTVHRHL |
| 29 | EDLTVKIGDFGLATEKSRWSGSHQFEQLS |
| 30 | TMKQICKKEIRRLHQY |
| 31 | RAHYNIVTF |
| 32 | MDMRVPAQLLGLLLLWLRGARC |
| 33 | QAEPDRAHYNIVTFCCKCD |
| 34 | ELFRAAQLANDVVLQIMEL |
| 35 | VHLELASMTNMELMSSIVH |
| 36 | LEQLESIINFEKLTEWTS |
| 37 | AESLKISQAVHAAHAEINEAGREVVGS |
| 38 | SAIRSYQYV |
| 39 | MALSTYYAL |
| 40 | AQLANDVVL |
| 41 | ASMTNMELM |
| 42 | SNFHFMCAL |
| 43 | QIYAFLQGF |
| 44 | FALMNLKAL |
| 45 | GVYPMPGTQK |
| 46 | GLLDEDFYA |
| 47 | KINKNPKYKK |
| 48 | SLADEAEVYL |
| 49 | VRTLLSQVNK |
| 50 | RFLEYLPLRF |
| 51 | KLKFVTLVF |
| 52 | DMKARQKALV |
| 53 | WLIRETQPITK |
| 54 | FPKKIQMLA |
| 55 | LRAAFFGKCF |
| 56 | YPVIFKSIM |
| 57 | VTEKLQPTY |
| 58 | WRNILLLSLH |
| 59 | EVLPFFLFF |
| 60 | QTNPVTLQY |
| 61 | TLYSLTLLY |
| 62 | FLIYLDVSV |
| 63 | FFYLLDFTF |
| 64 | IMQTLAGELY |
| 65 | TRSSGSHFVF |
| 66 | VLLGVKLFGV |
| 67 | ALYGFVPVL |
| 68 | ILTGLNYEV |
| 69 | FMPDFDLHL |
| 70 | TESPFEQHI |
| 71 | RLFPGLTIKI |
| 72 | CILGKLFTK |
| 73 | LTDDRLFTCY |
| 74 | GEEDGAGGHSL |
| 75 | GQFLTPNSH |
| 76 | RVSTLRVSL |

1. An immunogenic composition comprising a ribonucleic acid (RNA) molecule, said RNA molecule comprising an open-reading frame encoding a fusion protein, said fusion protein comprising
(i) a first polypeptide domain comprising either
a. an antigen or a fragment thereof comprising at least one epitope of said antigen,
b. a peptide moiety comprising a single epitope of an antigen, or
c. a plurality of peptide moieties, wherein each peptide moiety comprises an epitope of an antigen and wherein said peptide moieties are fused together, optionally via peptide linker,
said first polypeptide domain being fused, directly or indirectly via a peptide linker, to
(ii) a second polypeptide domain comprising a C4bp oligomerization domain and a positively charged tail.
wherein the second polypeptide domain is fused C-terminally to said first polypeptide domain, optionally via a peptide linker.

2. The immunogenic composition for use of claim 1, wherein said first polypeptide domain comprises a peptide moiety including (i) a CD8 or CD4 T cell epitope of said antigen and, optionally, (ii) the corresponding C-terminal and N-terminal natural flanking regions to said epitope, wherein each optional flanking region consists of 2 to 8 contiguous amino acid residues, typically 5 amino acid residues.

3. The immunogenic composition of claim 1, wherein said first polypeptide domain comprises a plurality of peptide moieties which are directly fused together or indirectly via peptide linker, and each peptide moiety comprises a fragment of an antigen including (i) an epitope of said antigen and (ii) the corresponding C-terminal and N-terminal natural flanking regions to said epitope, wherein each flanking region consists of 2 to 8 contiguous amino acid residues, typically 5 amino acid residues.

4. The immunogenic composition of claim 1, wherein said first polypeptide domain comprises at least two peptide moieties, each peptide moiety comprising an epitope of an antigen, for example a first peptide moiety comprising a CD8 T cell epitope of an antigen and a second peptide moiety comprising a CD4 T cell epitope of the same antigen.

5. The immunogenic composition of claim 1, wherein said RNA molecule encodes a fusion protein which consists essentially of
(i) a first polypeptide domain essentially consisting of a plurality of peptide moieties being fused together, optionally via peptide linker, each peptide moiety comprising an epitope of an antigen, wherein each peptide moiety essentially consists of a fragment of the antigen of 10 to 30 contiguous amino acid residues,
said first polypeptide domain being fused directly or indirectly via a peptide linker, to,
(ii) a second polypeptide domain essentially consisting of a C4bp oligomerization domain and a positively charged tail.

6. The immunogenic composition of claim 1, wherein said fusion protein further comprises a signal peptide, for example the Tissue plasminogen activator (tPA) signal sequence of SEQ ID NO:7.

7. The immunogenic composition of claim 1, wherein said antigen or said peptide moiety is selected from viral antigens or their fragments including at least one epitope, selected from the group consisting of the influenza nucleoprotein NP, coronavirus nucleocapsid N, coronavirus spike S, HPV E6 and E7.

8. The immunogenic composition of claim 1, wherein said fusion protein comprises one or more tumor mutation-derived neoepitope(s) or tumor associated antigen expressed in a tumor of a subject in need of said immunogenic composition.

9. The immunogenic composition of claim 8, wherein said fusion protein comprises either (i) one or more epitope(s) of tumor associated antigen(s) selected from the group consisting of MAGE-C1, MAGE-C2, NY-SEO-1, surviving, 5 T4, MUC1, PSA, PSCA, PSMA, STEAP1, PAP, MAGE-C3, tyrosinase, gp100, CT7, MAGE-A1, MAGE-A3, CEA, and HPV associated tumors, including E7 HPV antigen, or (ii) one or more tumor mutation-derived neoepitope(s) selected from beta catenin (CTNNB1) from melanoma, CDK4 from melanoma, alpha-actinin-4 (ACTN4) from non-small cell lung cancer, nuclear transcription factor γ subunit gamma from non-small cell lung cancer, Acyl-CoA:lysophosphatidylglycerol acyltransferase from bladder cancer, KRAS from Colorectal cancer, Protrudin (ZFYVE27) from pancreatic cancer, calcium-dependent secretion activator 2 (CASPS2) from breast cancer, B-RAF from melanoma, and dek-can fusion protein from myeloid leukemia.

10. The immunogenic composition of claim 1, wherein said self-assembling polypeptide derived from C4bp oligomerization domain comprises SEQ ID NO:2, or a functional variant thereof having at least 90% identity to SEQ ID NO:2.

11. The immunogenic composition of claim 1, wherein said positively charged tail comprises the sequence ZXBBBBZ (SEQ ID NO:3), wherein (i) Z is absent or is any amino acid, (ii) X is any amino acid, and (iii) B is an arginine or a lysine, preferably said positively charged tail comprises the sequence of SEQ ID NO:4.

12. The immunogenic composition of claim 1, wherein said carrier protein essentially consists of SEQ ID NO:5, or said carrier protein is a functional variant of SEQ ID NO:5 having at least 90% identity to SEQ ID NO:5.

13. The immunogenic composition of claim 1, wherein said RNA is a messenger RNA, preferably a synthetic mRNA molecule, comprising (i) a 5′ cap, (ii) a 5′ untranslated region (UTR), (iii) an open-reading frame that encode the fusion protein and (iv) a poly A tail.

14. The immunogenic composition of claim 1, wherein said RNA molecule is a nucleoside-modified RNA.

15. The immunogenic composition of claim 1, which is formulated in lipid nanoparticle (LNP).

16. A method for treating or preventing a cancer disorder in a subject in need thereof, said method comprising: administering to said subject an effective amount of an immunogenic composition of claim 1.

17. A method for treating or preventing an infectious disease in a subject in need thereof, said method comprising: administering to said subject an effective amount of an immunogenic composition of claim 1.

18. A method of inducing or increasing a CD8 or CD4 T cell response against a specific epitope in a subject in need thereof, said method comprising administering to said subject an effective amount of an immunogenic composition of claim 1.

19. The method of claim 18, wherein said CD8 or CD4 T cell response is directed against an epitope of a specific tumor associated antigen or tumor mutation derived neoepitope.

20. A method a treating a tumor in a subject in need thereof, said method comprising
a. identifying a specific neoepitope in the tumor of said subject,
b. in vitro transcribing or synthesizing an RNA molecule encoding a fusion protein, wherein said fusion protein comprises
(i) a first polypeptide domain comprising either
a. an antigen or a fragment thereof comprising at least one epitope of said antigen,
b. a peptide moiety comprising a single epitope of an antigen, or
c. a plurality of peptide moieties, wherein each peptide moiety comprises an epitope of an antigen and wherein said peptide moieties are fused together, optionally via peptide linker,
said first polypeptide domain being fused, directly or indirectly via a peptide linker, to
(ii) a second polypeptide domain comprising a C4bp oligomerization domain and a positively charged tail,
wherein said first polypeptide domain includes an amino acid sequence of at least said specific neoepitope,
c. producing an immunogenic composition comprising the in vitro transcribed or synthesized RNA molecule,
d. administering a therapeutically efficient amount of said immunogenic composition in a subject in need thereof.
